(19) United States
(12) Patent Application Publication
(10) Pub. No.: US 2026/0103512 A1
(43) Pub. Date: Apr. 16, 2026

(54) METHOD OF TREATING BONE CANCERS

(71) Applicant: AlaMab Therapeutics, Inc., Princeton, NJ (US)

(72) Inventors: Yanfeng Zhang, Phoenixville, PA (US); Xiugao Yang, Shanghai (CN)

(21) Appl. No.: 19/217,871

(22) Filed: May 23, 2025

Related U.S. Application Data
(60) Provisional application No. 63/651,826, filed on May 24, 2024.

(51) Int. Cl. C07K 16/28; A61K 39/00

(52) U.S. Cl. CPC C07K 16/28; A61K 2039/505; A61K 2039/545; A61K 2039/55; A61K 2039/80; C07K 2317/24

(57) ABSTRACT

The present disclosure provides compositions and methods of treating bone cancers, e.g., osteosarcoma in a patient whose age is, for example, at least 2 years old, or at least 12 years old. The method comprises promoting the opening of Cx43 hemichannel in osteocytes by, e.g., using a composition comprising an anti-connexin 43 antibody.

### CROSS REFERENCE TO RELATED APPLICATIONS

This application claims the benefit of U.S. Provisional Application No. 63/651,826, filed May 24, 2024, the entire disclosure of which is incorporated herein by reference.

### SEQUENCE LISTING

The instant application contains a Sequence Listing, which has been submitted via Patent Center. The Sequence Listing titled 172628-204003_US.xml, which was created on Apr. 10, 2025 and is 14,428 bytes in size, is hereby incorporated by reference in its entirety.

### BACKGROUND

Osteosarcoma is the most common primary malignant bone tumor derived from primitive bone-forming mesenchymal stem cells. Osteosarcoma has a bimodal age distribution, with the first peak in adolescents aged 10-14 years and the second peak in adults aged >65 years. It generally occurred more in males than in females, 5.4/million/year in men vs. 4.0/million/year in women, and more in black people (6.8/million) and Hispanics (6.5/million) than in white people (4.6/million).

Osteosarcoma usually occurs in the long bones of the extremities close to the bony end of the metaphyseal growth plate. Of all children and adolescent cancer death, 8.9% died from bone and joint malignancies. The 5-year survival rate for osteosarcoma was 68%, without significant gender differences. The expected 5-year survival rate for metastatic patients is 15%-30% (Lin et al., Trends in molecular medicine, 23 (8): 737-755 (2017)), and the survival rate of patients is age-related, with older patients having a lower survival rate. The prognosis of osteosarcoma is very poor. According to statistics, the 5-year and 10-year overall survival is 0.23 and 0.18, respectively (Kempf-Bielack et al., J Clin Oncol., 23: 559-568 (2005)). Tumor staging, metastasis, local recurrence, chemotherapy regimen, anatomical location, tumor size, and tumor cell destruction rate after neoadjuvant chemotherapy are all associated with the results of treatment (Ottaviani et al., Cancer Treat Res., 152: 3-13 (2009)).

For high-grade osteosarcoma (including intramedullary and surface forms), preoperative chemotherapy is recommended (Grade 1A), followed by reassessment and re-staging by chest radiography, local X-ray, PET, or bone scan. For resectable tumors, wide resection should be performed. When the resection margin is negative and the response to chemotherapy is good, chemotherapy should be continued; when the response to chemotherapy is poor, the chemotherapy regimen can be modified. When the resection margin is positive and the response to chemotherapy is good, chemotherapy should be continued, and other local treatments (surgery, radiotherapy, etc.) may be performed concomitantly; when the response to chemotherapy is poor, chemotherapy regimen can be modified, and other local treatments (surgery, radiotherapy, etc.) should be performed concomitantly. For unresectable tumors, only radiotherapy and chemotherapy are considered. Patients should be continuously monitored after treatment.

Osteocytes have high expression of connexin Cx43 in interstitial junction channels and hemichannels. The gap junction channels mediate cellular communication between two adjacent cells to maintain the normal metabolic function of the cells, while the hemichannels allow molecular exchange between the cells and the extracellular environment. Hemichannels are normally closed, but under certain conditions, such as mechanical stress or ischemia, cytokine or ion stimulation, they will be activated and opened. Opened hemichannels allow small molecules (<1.2 kDa) such as ATP, NAD+, glutamic acid, and inositol triphosphate (IP3) to pass (Goodenough et al., Nature reviews Molecular cell biology, 4: 285-294 (2003)). It has been confirmed that the opening of the Cx43 hemichannels of osteocytes has a strong inhibitory effect on the growth, migration and bone metastasis of breast cancer tumors (Zhou et al., Oncogene, 35: 5597-5607 (2016)).

Current first-line chemotherapeutics for osteosarcoma are methotrexate, doxorubicin, cisplatin, and ifosfamide. As recommended by CSCO in 2020, since there is no second-line treatment regimen showing overall survival benefit, participation in clinical studies is an opportunity for osteosarcoma patients who fail first-line treatments to achieve better efficacy or to renew treatment. It has been reported in the literature that a drug is considered effective in clinical studies if it provides a response rate of >5% or a 4-month PFS rate of >40%. Systemic chemotherapy is the primary treatment for advanced osteosarcoma, and no major breakthrough has been made in the treatment of advanced osteosarcoma in the past decades. As of now, there remains a large and unmet need for medication in advanced osteosarcoma.

### SUMMARY

One aspect of the present disclosure provides a method of treating a bone cancer in a subject in need thereof, and at least 2 years old and less than 18 years old, the method comprises administering to the subject at least one dose of an anti-Cx43 antibody, or antigen binding fragment thereof, the anti-Cx43 antibody comprises a heavy chain variable region comprising an HCDR1 having the amino acid sequence of SEQ ID NO 1, an HCDR2 having the amino acid sequence of SEQ ID NO 2, and an HCDR3 having the amino acid sequence of SEQ ID NO 3; and a light chain variable region comprising an LCDR1 having the amino acid sequence of SEQ ID NO 4, an LCDR2 having the amino acid sequence of SEQ ID NO 5, and an LCDR3 having the amino acid sequence of SEQ ID NO 6.

In some embodiments, the subject is at least 3 years old, at least 4 years old, at least 5 years old, at least 6 years old, at least 7 years old, at least 8 years old, at least 9 years old, at least 10 years old, at least 11 years old, at least 12 years old, at least 13 years old, at least 14 years old, at least 15 years old, at least 16 years old, or at least 17 years old.

In some embodiments, the subject is at least 12 years old, and less than 18 years old.

In some embodiments, the at least one dose of the anti-Cx43 antibody, or antigen binding fragment thereof, is about 0.001 mg/kg to about 300 mg/kg. In some embodiments, the at least one dose of the anti-Cx43 antibody, or antigen binding fragment thereof, is about 1 mg/kg, about 3 mg/kg, about 6 mg/kg, about 12 mg/kg, about 18 mg/kg, about 24 mg/kg, about 30 mg/kg, or about 36 mg/kg.

In some embodiments, the anti-Cx43 antibody, or antigen binding fragment thereof, is administered about once every day to about once every 12 months. In some embodiments, the anti-Cx43 antibody, or antigen binding fragment thereof, is administered about once every 2 weeks, about once every 3 weeks, or about once every 4 weeks. In some embodiments, the anti-Cx43 antibody, or antigen binding fragment thereof, is administered about once every 3 weeks.

In some embodiments, the anti-Cx43 antibody, or antigen binding fragment thereof, is administered intravenously, intradermally, intratumorally, intramuscularly, intraperitoneally, or subcutaneously. In some embodiments, the anti-Cx43 antibody, or antigen binding fragment thereof, is administered intravenously.

In some embodiments, the administration of the anti-Cx43 antibody, or antigen binding fragment thereof, is completed within about 5 minutes, about 10 minutes, about 15 minutes, about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more.

In some embodiments, the anti-Cx43 antibody, or antigen binding fragment thereof, is administered in a pharmaceutically acceptable composition. In some embodiments, the pharmaceutically acceptable composition comprises a pharmaceutically acceptable carrier.

In some embodiments, the anti-Cx43 antibody, or antigen binding fragment thereof, comprises a heavy chain variable region comprising the amino acid sequence of SEQ ID NO 7, and/or a light chain variable region comprising the amino acid sequence of SEQ ID NO 8.

In some embodiments, the anti-Cx43 antibody comprises a heavy chain comprising the amino acid sequence of any one of SEQ ID NOS 9 and 11-13, and/or a light chain comprising the amino acid sequence of SEQ ID NO 10.

In some embodiments, the anti-Cx43 antibody is a humanized antibody.

In some embodiments, the anti-Cx43 antibody, or antigen binding fragment thereof, enhances Cx43 hemichannel opening in the subject.

In some embodiments, the bone cancer is osteosarcoma. In some embodiments, the osteosarcoma is (i) resectable, or unresectable; (ii) stage I-A, stage I-B, stage II-A, stage II-B, stage III, stage IV-A, or stage IV-B; (iii) low grade, or high grade; and/or (iv) primary osteosarcoma, osteoblastic osteosarcoma, chondroblastic osteosarcoma, fibroblastic osteosarcoma, small cell osteosarcoma, telangiectatic osteosarcoma, parosteal osteosarcoma, periosteal osteosarcoma, classic osteosarcoma, osteoblastic osteosarcoma-sclerosing type osteosarcoma, chondroblastoma-like osteosarcoma, chondromyxoid fibroma-like osteosarcoma, clear-cell osteosarcoma, malignant fibrous histiocytoma-like osteosarcoma, giant cell rich osteosarcoma, and/or epithelioid osteosarcoma.

In some embodiments, the subject had failed treatment with at least one standard of care for osteosarcoma comprising chemotherapy, surgery, immune therapy, or radiotherapy, or a combination thereof.

In some embodiments, the subject had failed treatment with methotrexate, doxorubicin, cisplatin, and ifosfamide, apatinib, anlotinib, vindesine, vincristine, docetaxel, paclitaxel, irinotecan, bortezomib, albumin-bound paclitaxel, nedaplatin (Aqupla), pemetrexed, etoposide, gemcitabine, lobaplatin, recombinant human endostatin, eribulin, dacarbazine, pazopanib, immune checkpoint inhibitors, surgery, or radiotherapy, or the combination thereof.

In some embodiments, after treatment with the anti-Cx43 antibody, or antigen binding fragment thereof, the subject achieves: (i) progression-free survival for at least 1 month, 2 months, 3 months, at least 4 months, at least 5 months, or at least 6 months; (ii) improved overall survival by at least 1 month, at least 2 months, at least 3 months, at least 4 months, at least 5 months, at least 6 months; (iii) improved EQ-5D score; (iv) improved disease progression as assessed by PERCIST criteria, RECIST criteria, and/or ICDS criteria; (v) improved pain score as assessed by NRS; and/or (vi) reduced tumor size by at least 1%, at least 2%, at least 3%, at least 5%, at least 8%, at least 10%, at least 15%, at least 20%, or at least 30%.

In some embodiments, the subject achieves complete or partial response to the treatment 20 with the anti-Cx43 antibody, or antigen binding fragment thereof.

Another aspect of the disclosure provides a method of treating a bone cancer in a subject in need thereof, and at least 12 years old and less than 18 years old, the method comprises administering to the subject at least one dose of an anti-Cx43 antibody, or antigen binding fragment thereof, wherein the anti-Cx43 antibody comprises a heavy chain variable region comprising an HCDR1 having the amino acid sequence of SEQ ID NO 1, an HCDR2 having the amino acid sequence of SEQ ID NO 2, and an HCDR3 having the amino acid sequence of SEQ ID NO 3; and a light chain variable region comprising an LCDR1 having the amino acid sequence of SEQ ID NO 4, an LCDR2 having the amino acid sequence of SEQ ID NO 5, and an LCDR3 having the amino acid sequence of SEQ ID NO 6.

In some embodiments, the at least one dose of the anti-Cx43 antibody, or antigen binding fragment thereof, is about 0.001 mg/kg to about 300 mg/kg. In some embodiments, the at least one dose of the anti-Cx43 antibody, or antigen binding fragment thereof, is about 1 mg/kg, about 3 mg/kg, about 6 mg/kg, about 12 mg/kg, about 18 mg/kg, about 24 mg/kg, about 30 mg/kg, or about 36 mg/kg.

In some embodiments, the anti-Cx43 antibody, or antigen binding fragment thereof, is administered about once every day to about once every 12 months. In some embodiments, the anti-Cx43 antibody, or antigen binding fragment thereof, is administered about once every 2 weeks, about once every 3 weeks, or about once every 4 weeks. In some embodiments, the anti-Cx43 antibody, or antigen binding fragment thereof, is administered about once every 3 weeks.

In some embodiments, the anti-Cx43 antibody, or antigen binding fragment thereof, is administered intravenously, intradermally, intratumorally, intramuscularly, intraperitoneally, or subcutaneously. In some embodiments, the anti-Cx43 antibody, or antigen binding fragment thereof, is administered intravenously.

In some embodiments, the administration of the anti-Cx43 antibody, or antigen binding fragment thereof, is completed within about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more.

In some embodiments, the anti-Cx43 antibody, or antigen binding fragment thereof, is administered in a pharmaceutically acceptable composition. In some embodiments, the pharmaceutically acceptable composition comprises a pharmaceutically acceptable carrier.

In some embodiments, the anti-Cx43 antibody, or antigen binding fragment thereof, comprises a heavy chain variable region comprising the amino acid sequence of SEQ ID NO 7, and/or a light chain variable region comprising the amino acid sequence of SEQ ID NO 8.

In some embodiments, the anti-Cx43 antibody comprises a heavy chain comprising the amino acid sequence of any one of SEQ ID NOS 9 and 11-13, and/or a light chain comprising the amino acid sequence of SEQ ID NO 10.

In some embodiments, the anti-Cx43 antibody is a humanized antibody.

In some embodiments, the anti-Cx43 antibody, or antigen binding fragment thereof, enhances Cx43 hemichannel opening in the subject.

In some embodiments, the bone cancer is osteosarcoma. In some embodiments, the osteosarcoma is (i) resectable, or unresectable; (ii) stage I-A, stage I-B, stage II-A, stage II-B, stage III, stage IV-A, or stage IV-B; (iii) low grade, or high grade; and/or (iv) primary osteosarcoma, osteoblastic osteosarcoma, chondroblastic osteosarcoma, fibroblastic osteosarcoma, small cell osteosarcoma, telangiectatic osteosarcoma, parosteal osteosarcoma, periosteal osteosarcoma, classic osteosarcoma, osteoblastic osteosarcoma-sclerosing type osteosarcoma, chondroblastoma-like osteosarcoma, chondromyxoid fibroma-like osteosarcoma, clear-cell osteosarcoma, malignant fibrous histiocytoma-like osteosarcoma, giant cell rich osteosarcoma, and/or epithelioid osteosarcoma.

In some embodiments, the subject had failed treatment with at least one standard care of osteosarcoma comprising chemotherapy, surgery, immune therapy, or radiotherapy, or a combination thereof. In some embodiments, the subject had failed treatment with methotrexate, doxorubicin, cisplatin, and ifosfamide, apatinib, anlotinib, vindesine, vincristine, docetaxel, paclitaxel, irinotecan, bortezomib, albumin-bound paclitaxel, nedaplatin (Aqupla), pemetrexed, etoposide, gemcitabine, lobaplatin, recombinant human endostatin, eribulin, dacarbazine, pazopanib, immune checkpoint inhibitors, surgery, or radiotherapy, or the combination thereof.

In some embodiments, after treatment with the anti-Cx43 antibody, or antigen binding fragment thereof, the subject achieves: (i) progression-free survival for at least 1 month, 2 months, 3 months, at least 4 months, at least 5 months, or at least 6 months; (ii) improved overall survival by at least 1 month, at least 2 months, at least 3 months, at least 4 months, at least 5 months, at least 6 months; (iii) improved EQ-5D score; (iv) improved disease progression as assessed by PERCIST criteria, RECIST criteria, and/or ICDS criteria; (v) improved pain score as assessed by NRS; and/or (vi) reduced tumor size by at least 1%, at least 2%, at least 3%, at least 5%, at least 8%, at least 10%, at least 15%, at least 20%, or at least 30%.

In some embodiments, the subject achieves complete or partial response to the treatment with the anti-Cx43 antibody, or antigen binding fragment thereof.

### BRIEF DESCRIPTION OF DRAWINGS

FIG. 1 shows the pain grades of Numeric Rating Scale.

FIG. 2 shows the VAS scale of the EQ-5D questionnaire. As stated in the EQ-5D questionnaire, to reflect your health, we drew a scale (kind of like a thermometer) in which 100 represents the best in your mind and 0 represents the worst in your mind. Please mark your own health status today on the scale. Please draw a line from the box below to the point on the scale that best represents your health today.

### DETAILED DESCRIPTION

The present disclosure provides methods and compositions for treating a cancer, e.g., bone cancer, in a subject or patent in need thereof who is at least 2 years old or at least 12 years old, comprising administering at least one dose of an anti-Cx43 antibody to the subject or patent. In some embodiments, the anti-Cx43 antibody promotes the opening of Cx43 hemichannel. In some embodiments, the anti-Cx43 antibody comprises specific CDR amino acid sequences. In some embodiments, the anti-Cx43 antibody is administered according to a dosing regimen.

### I. Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% of a reference quantity, level, value, number, frequency, percentage, dimension, size, amount, weight or length.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

Throughout this specification, unless the context requires otherwise, the words “comprise,” “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “Adverse Event” or “AE” refers to any adverse medical event in a subject administered with a drug, which may be manifested as symptoms, signs, diseases, or abnormal laboratory tests, but not necessarily causally related to the drug.

As used herein, the term “affinity” refers to the strength of a binding reaction between a binding domain of an antibody and an epitope. It is the sum of the attractive and repulsive forces operating between the binding domain and the epitope. The term affinity, as used herein, refers to the dissociation constant Kd.

As used herein, the term “antibody” or “antibodies” refers to a full length antibody (also known as immunoglobulin), antibody peptide(s), monoclonal antibodies, chimeric antibodies, polyclonal antibodies, human antibodies, humanized antibodies and antibodies from non-human species, including human antibodies derived from a human germline immunoglobulin sequence transduced into the non-human species, e.g., mouse, sheep, chicken or goat, recombinant antigen binding forms such as monobodies and diabodies, multispecific antibodies (e.g., bispecific antibodies) formed from at least two full length antibodies (e.g., each portion comprising the antigen binding region of an antibody to a different antigen or epitope), and individual antigen binding fragments of any of the foregoing, e.g., of an antibody or the antibody from which it is derived, including dAbs, Fv, scFv, Fab, F(ab)′2, Fab′. Antibody-like binding peptidomimetics are also contemplated in the methods described herein. Liu et al. (2003) describe “antibody-like binding peptidomimetics” (ABiPs), which are peptides that act as pared-down antibodies and have certain advantages of longer serum half-life as well as less cumbersome synthesis methods.

As used herein, the term “bone cancer” or “bone tumor” refers to any cancer related to bone, including a cancer originated from a bone, or a cancer originated from other organs or tissues, (e.g., breast, prostate, lung, kidney, and thyroid etc.) and metastasizes to a bone.

Accordingly, non-limiting examples of a bone cancer include osteosarcoma, multiple myeloma, Ewing's sarcoma, osteoporosis, osteopenia, osteoblastoma, chondroma, chondromyxoid fibroma, mesenchymal chondrosarcoma, chordoma, adamantinoma, or bone cancer metastasis.

As used herein, the term “antigen binding fragments” is the portion of a variable region that retains antigen binding activity. An antigen binding fragment can include, for example, the variable regions of the heavy and/or light chains of an anti-Cx43 antibody and can also be shorter than the variable regions of the heavy and/or light chains of an anti-Cx43 antibody. For example, an antigen binding fragment of anti-Cx43 antibodies can comprise the amino acid sequences of SEQ ID NOS 7 and 8. Examples of such antigen binding fragments include Fab fragments, Fab′ fragments, Fv fragments, scFv and F(ab′)2 fragments.

As used herein, the term “Fab” or “Fab region” is meant the polypeptide that comprises the VH, CH1, VL, and CL immunoglobulin domains. Fab may refer to this region in isolation, or this region in the context of a full-length antibody, antibody fragment or Fab fusion protein, or any other antibody embodiments as outlined herein.

As used herein, the term “Fv,” “Fv fragment” or “Fv region” refers to a polypeptide that comprises the VL and VH domains of a single antibody.

As used herein, the term “framework” refers to the region of an antibody variable domain exclusive of those regions defined as CDRs. Each antibody variable domain framework can be further subdivided into the contiguous regions separated by the CD Rs (FR1, FR2, FR3 and FR4).

As used herein, “humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human antibody are replaced by corresponding non-human residues. Furthermore, humanized antibodies may comprise residues that are not found in the recipient antibody or in the donor antibody. These modifications are made to further refine antibody performance. For further details, see Jones et al., Nature, 321:522-525 (1986); Riechmann et al., Nature, 332:323-329 (1988); and Presta, Curr. Op. Struct. Biol, 2:593-596 (1992).

As used herein, the term “hypervariable region” refers to the amino acid residues of an antibody which are responsible for antigen binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mal. Biol., 196:901-917 (1987)). “Framework Region” or “FR” residues are those variable domain residues other than the hypervariable region residues as herein defined. The hypervariable region or the CDRs thereof can be transferred from one antibody chain to another or to another protein to confer antigen binding specificity to the resulting (composite) antibody or binding protein. As will be appreciated by those in the art, the CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent CDR of the invention, and shares biological function, including, but not limited to, at least 80%, 81%, 82%, 83%, 84%, 85%, 86%, 87%, 88%, 89%, 90%, 91% 92%, 93%, 94%, 95%, 96%, 97%, 98%, or 99% of the specificity and/or activity of the parent CDR. Amino acid substitutions are typically of single residues; insertions usually will be on the order of from about one to about twenty amino acid residues, although considerably larger insertions may be tolerated. Deletions range from about one to about twenty amino acid residues, although in some cases deletions may be much larger. Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

As used herein, the term “isolated” antibody refers to an antibody that has been identified and separated and/or recovered from a component of its natural environment. In certain embodiments, the antibody will be purified (1) to greater than 95% by weight of protein as determined by the Lowry method, and alternatively, more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

As used herein, the term “monoclonal antibody” refers to an antibody obtained from a population of substantially homogeneous antibodies, i.e., the individual antibodies comprising the population are identical and/or bind the same epitope. The modifier “monoclonal” indicates the character of the antibody as being obtained from a substantially homogeneous population of antibodies, and is not to be construed as requiring production of the antibody by any particular method.

As used herein, the term “cancer” or “tumor” is intended to include any malignant or neoplastic growth in a patient, including an initial tumor and any metastases. The cancer can be of the hematological or solid tumor type. Hematological tumors include tumors of hematological origin, including, e.g., myelomas (e.g., multiple myeloma), leukemias (e.g., Waldenstrom's syndrome, chronic lymphocytic leukemia, acute myelogenous leukemia, chronic myelogenous leukemia, granulocytic leukemia, monocytic leukemia, acute lymphocytic leukemia, other leukemias), lymphomas (e.g., B-cell lymphomas, such as diffuse large B-cell lymphoma, follicular lymphoma, mantle cell lymphoma, Hodgkin's lymphoma, non-Hodgkin's lymphoma, plasmacytoma, or reticulum cell sarcoma), and myeloproliferative neoplasms, such as myelodysplastic syndrome, thrombocythemia, polycythemia vera, or myelofibrosis. Solid tumors can originate in organs, and include cancers such as in bone, skin, lung, brain, breast, prostate, ovary, colon, kidney, pancreas, liver, esophagus, stomach, intestine, bladder, uterus, cervix, testis, adrenal gland, etc. As used herein, cancer cells, including tumor cells, refer to cells that divide at an abnormal (increased) rate or whose control of growth or survival is different than for cells in the same tissue where the cancer cell arises or lives. Cancer cells include, but are not limited to, cells in carcinomas, sarcomas, myelomas, leukemias, lymphomas, and tumors of the nervous system including glioma, meningioma, medulloblastoma, schwannoma or ependymoma.

In a preferred embodiment, the cancer is bone cancer. In a more preferred embodiment, the bone cancer is osteosarcoma.

As used herein, the term “effective amount” refers to that amount of an agent, such as an 10 anti-Cx43 antibody, which is sufficient to effect treatment, prognosis or diagnosis of a cancer (e.g., bone cancer), when administered to a patient or a subject. Dosage regimens may be adjusted to provide the optimum therapeutic response. An effective amount is also one in which any toxic or detrimental effects (side effects) of the agent are minimized and/or outweighed by the beneficial effects. A therapeutically effective amount will vary depending upon the patient and disease condition being treated, the weight and age of the patient, the severity of the disease condition, the manner of administration, course of the condition, patient's clinical history and response to anti-Cx43 antibody and the like, which can readily be determined by one of ordinary skill in the art.

As used herein, the term “parenteral administration” and “administered parenterally” refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Intravenous injection and infusion are often (but not exclusively) used for antibody administration.

As used herein, the term “pediatric subject” refers to a subject having an age of less than 18 years old. A pediatric subject can have an age of 2 years old, or 3 years old, 4 years old, 5 years old, 6 years old, 7 years old, 8 years old, 9 years old, 10 years old, 11 years old, 12 years old, 13 years old, 14 years old, 15 years old, 16 years old, or 17 years old.

As used herein, the terms “peptide,” “polypeptide,” and “protein” are used interchangeably and refer to a molecule having amino acid residues covalently linked by peptide bonds. A polypeptide must contain at least two amino acids, and no limitation is placed on the maximum number of amino acids of a polypeptide. As used herein, the terms refer to both short chains, which are also commonly referred to in the art as peptides, oligopeptides and oligomers, for example, and to longer chains, which generally are referred to in the art as polypeptides or proteins. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural polypeptides, recombinant polypeptides, synthetic polypeptides, or a combination thereof.

As used herein, the term “percent sequence identity” or “sequence identity” refers to the degree of identity between any given query sequence and a subject sequence. A percent identity for any query nucleic acid or amino acid sequence, relative to another subject nucleic acid or amino acid sequence can be determined using tools and technologies known in the art, for example, NCBI BLAST.

As used herein, the term “pharmaceutical formulation” or “pharmaceutical composition” refers to a preparation that contains a therapeutic agent (e.g., an anti-Cx43 antibody). In such form as to permit the biological activity of the antibody to be effective, and which contains no additional components which are unacceptably toxic to a subject to which the formulation would be administered.

As used herein, the term “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent may be coated m a material to protect the compm.md from the action of acids and other natural conditions that may inactivate the compound.

As used herein, the terms “promote,” “enhance” and “induce” refer to any statistically significant increase in biological activity (e.g., hemichannel opening). For example, “promotion” can refer to an increase of about 10%, 20%, 30%, 40%, 50%, 60%, 70%, 80%, 90%, or 100% in biological activity.

As used herein, the term “providing” is used according to its ordinary meaning to supply or furnish for use. In some embodiments, the antibody or antigen binding fragment thereof is provided directly by administering the protein, while in other embodiments, the antibody or antigen binding fragment thereof is effectively provided by administering a nucleic acid that encodes the protein. In certain aspects the invention contemplates compositions comprising various combinations of nucleic acid, antigens, peptides, and/or epitopes.

As used herein, the term “specifically binds” is not intended to indicate that an antibody binds exclusively to its intended target. Rather, an antibody “specifically binds” if its affinity for its intended target is about 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some embodiments, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹. Antibodies may, for example, bind with a Kd of 50 nM or less, 10 nM or less, 1 nM or less, 100 pM or less, or more preferably 10 pM or less. As known in the art, a variety of immunoassay formats may be used to select antibodies specifically immunoreactive with an antigen. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

As used herein, the term “therapeutic monoclonal antibody” is an antibody used for therapy of a human subject. Therapeutic monoclonal antibodies disclosed herein include anti-Cx43 antibodies. Antibody “effector functions” refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), and the like.

To assess ADCC activity of a molecule of interest, an in vitro ADCC assay, such as those described in U.S. Pat. No. 5,500,362 or U.S. Pat. No. 5,821,337 may be performed. Depending on the amino acid sequence of the constant domain of their heavy chains, full length antibodies can be assigned to different “classes.” There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ and μ, respectively. The subunit structures and three-dimensional configurations of different classes of antibodies are well known. The “light chains” of antibodies from any vertebrate species can be assigned to one of two clearly distinct types, called kappa (κ) and lambda (λ), based on the amino acid sequences of their constant domains. Moieties of the invention, such as polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

As used herein, the term “sequentially administering” means that each dose of anti-Cx43 antibody is administered to the subject at a different point in time, e.g., on different days separated by a predetermined interval (e.g., hours, days, weeks or months). The present disclosure includes methods which comprise sequentially administering to the patient a single initial dose of an anti-Cx43 antibody, followed by one or more secondary doses of the anti-Cx43 antibody, and optionally followed by one or more tertiary doses of the anti-Cx43 antibody. The anti-Cx43 antibody may be administered at a dose of between 0.001 mg/kg to about 300 mg/kg.

As used herein, the terms “initial dose,” “secondary doses,” and “tertiary doses” refer to the temporal sequence of administration of the anti-Cx43 antibody of the disclosure. Thus, the “initial dose” is the dose which is administered at the beginning of the treatment regimen (also referred to as the “baseline dose”); the “secondary dose” are the doses which are administered after the initial dose; and the “tertiary doses” are the doses which are administered after the secondary doses. The initial, secondary, and tertiary doses may all contain the same amount of anti-Cx43 antibody, but generally may differ from one another in terms of frequency of administration. In certain embodiments, however, the amount of anti-Cx43 antibody contained in the initial, secondary, tertiary and/or doses following the tertiary dose varies from one another (e.g., adjusted up or down as appropriate) during the course of treatment.

### II. Osteosarcoma

Osteosarcoma is the most common primary malignant bone tumor. By definition, osteosarcoma is an osteoid-forming spindle cell neoplasm with several histologic subtypes demonstrating variable radiographic appearances, intrinsic cellular heterogeneity, and overall prognosis. Subtypes of osteosarcoma include but are not limited to: osteoblastic osteosarcoma, chondroblastic osteosarcoma, fibroblastic osteosarcoma, small cell osteosarcoma, telangiectatic osteosarcoma, parosteal osteosarcoma, periosteal osteosarcoma, classic osteosarcoma, osteoblastic osteosarcoma-sclerosing type osteosarcoma, chondroblastoma-like osteosarcoma, a chondromyxoid fibroma-like osteosarcoma, clear-cell osteosarcoma, malignant fibrous histiocytoma-like osteosarcoma, giant cell rich osteosarcoma, or epithelioid osteosarcoma. he World Health Organizations considers osteoblastic, chondroblastic and fibroblastic subtypes of conventional intramedullary osteosarcoma, the most common type of osteosarcoma.

Osteoblastic osteosarcoma arises from abnormal proliferation of osteoblasts. Osteoblastic osteosarcoma typically develops in the metaphyseal region of long bones, such as the distal femur, proximal tibia, proximal humerus etc., but it can also occur in other bones. Osteoblastic osteosarcoma is characterized in the setting of abundant extracellular osteoid production. The matrix comprised of immature bone is deposited in a lace-like pattern with malignant tumor cells incorporated throughout the extracellular matrix.

Chondroblastic osteosarcoma as defined by WHO is a histological entity characterized by predominant presence of chondroid matrix, which tends to exhibit a high degree of hyaline cartilage and is intimately associated with the nonchondroid element (osteoid or bone matrix). Chondroblastic osteosarcoma typically occurs in the long bones, such as the femur and tibia. It can also affect other bones, including the humerus and pelvis. Chondroblastic osteosarcoma is more common in adolescents and young adults, but it can occur at any age.

Fibroblastic osteosarcomas are characterized by the presence of spindle-shaped cells resembling fibroblasts. Fibroblastic osteosarcomas are highly vascular and may resemble hemangiopericytoma. In histopathology, osteoid production is minimal and seen focally.

Small cell osteosarcoma, characterized by the presence of small, round, and uniform cells within the tumor, is a rare subtype of osteosarcoma. In histopathology, small cell osteosarcoma cells have scant cytoplasm and large, hyperchromatic nuclei. small cell osteosarcoma mainly occurs in the metaphyseal region of long bones.

Benign giant cells resembling osteoclasts are seen in about 25% of osteosarcomas. In rare osteosarcomas, benign giant cells are so numerous that they may obscure the malignant cells in the background and lead to the mistaken diagnosis of a giant cell tumor. This is more likely to occur in sacrum—a frequent location for giant cell tumor. The radiographic differences between the giant cell tumor and osteosarcoma are less apparent in the sacrum than in the long bones. It is worth remembering that usual giant cell tumor occurs in skeletally mature patients. A tumor that appears to be a giant cell tumor but arising in a skeletally immature person should be sampled thoroughly and carefully to exclude osteosarcoma rich in giant cells.

There are many classification systems for grading osteosarcoma. One of the widely used system is the 4-tiered (Broder's) system based on the percentage of anaplasia in the tumor. Majority of the conventional central osteosarcomas fall in grade 3 or grade 4 category. The tumors assigned grade 1 are low-grade central osteosarcomas and the usual parosteal osteosarcomas. Osteosarcomas of the jaw often tend to show grade 2 histology.

The common staging systems for malignant bone tumors are: Enneking system for staging malignant musculoskeletal tumors and the American Joint Committee on Cancer (AJCC) System for staging osteosarcomas. Enneking system is based on histological grade of the tumor, its local extent and the presence or absence of metastasis. The AJCC system for bone sarcomas is based on tumor grade, size, presence, and location of metastases. Different stages of the AJCC system are shown below.

| Stage | Grade | Size | Metastasis |
|---|---|---|---|
| I-A | Low | <8 cm | None |
| I-B | Low | >8 cm | None |
| II-A | High | <8 cm | None |
| II-B | High | >8 cm | None |
| III | Any | Any | Skip metastasis |
| IV-A | Any | Any | Pulmonary metastasis |
| IV-B | Any | Any | Non-Pulmonary metastasis |

### III. Anti-Cx43 Antibodies

Certain aspects of the present disclosure provide compositions and methods of treating a bone cancer, e.g., osteosarcoma, using an anti-Cx43 antibody, or an antigen binding fragment thereof, that specifically binds Cx43 hemichannel and promotes the opening of it in osteocytes.

Antigen binding fragments of an antibody can be produced by enzymatic cleavage or by recombinant techniques. For instance, papain or pepsin cleavage can be used to generate Fab or F(ab′)2 fragments, respectively. Antibodies can also be produced in a variety of truncated forms using antibody genes in which one or more stop codons have been introduced upstream of the natural stop site. For example, a recombinant construct encoding the heavy chain of an F(ab′)2 fragment can be designed to include DNA sequences encoding the CH1 domain and hinge region of the heavy chain. In one aspect, antigen binding fragments promote opening of Cx43 hemi-channel in a subject and the effects associated with opening of Cx43 hemi-channel.

The anti-Cx43 antibody can be any antibody that specifically binds Cx43 as known in the art. In some embodiments, the anti-Cx43 antibody used in the methods contains an HCDR1 amino acid sequence having at least 90%, at least 95%, or at least 99% sequence identity to SEQ ID NO 1. In some embodiments, the anti-Cx43 antibody contains an HCDR2 amino acid sequence having at least 90%, at least 95%, or at least 99% sequence identity to SEQ ID NO 2. In some embodiments, the anti-Cx43 antibody contains an HCDR3 amino acid sequence having at least 90%, at least 95%, or at least 99% sequence identity to SEQ ID NO 3. In some embodiments, the anti-Cx43 antibody contains an LCDR1 amino acid sequence having at least 90%, at least 95%, or at least 99% sequence identity to SEQ ID NO 4. In some embodiments, the anti-Cx43 antibody contains an LCDR2 amino acid sequence having at least 90%, at least 95%, or at least 99% sequence identity to SEQ ID NO 5. In some embodiments, the anti-Cx43 antibody contains an LCDR3 amino acid sequence having at least 90%, at least 95%, or at least 99% sequence identity to SEQ ID NO 6.

In some embodiments, the anti-Cx43 antibody contains an HCDR1 amino acid sequence identical to SEQ ID NO 1, an HCDR2 amino acid sequence identical to SEQ ID NO 2, and/or an HCDR3 amino acid sequence identical to SEQ ID NO 3. In some embodiments, the anti-Cx43 antibody contains an LCDR1 amino acid sequence identical to SEQ ID NO 4, an LCDR1 amino acid sequence identical to SEQ ID NO 5, and an LCDR3 amino acid sequence identical to SEQ ID NO 6.

In some embodiments, the anti-Cx43 antibody contains an HCDR1 amino acid sequence identical to SEQ ID NO 1, an HCDR2 amino acid sequence identical to SEQ ID NO 2, and an HCDR3 amino acid sequence identical to SEQ ID NO 3, and an LCDR1 amino acid sequence identical to SEQ ID NO 4, an LCDR1 amino acid sequence identical to SEQ ID NO 5, and an LCDR3 amino acid sequence identical to SEQ ID NO 6.

In some embodiments, the anti-Cx43 antibody used in the method contains a heavy chain variable region having an amino acid sequence that has at least 80%, at least 85%, at least 90%, at least 95%, or at least 99% sequence identity to SEQ ID NO 7. In some embodiments, the anti-Cx43 antibody contains a light chain variable region having an amino acid sequence that has at least 80%, at least 85%, at least 90%, at least 95%, or at least 99% sequence identity to SEQ ID NO 8. In some embodiments, the anti-Cx43 antibody contains a heavy chain variable region having the amino acid sequence of SEQ ID NO 7. In some embodiments, the anti-Cx43 antibody contains a light chain variable region having the amino acid sequence of SEQ ID NO 8. In some embodiments, the anti-Cx43 antibody contains a heavy chain variable region having the amino acid sequence of SEQ ID NO 7, and a light chain variable region having the amino acid sequence of SEQ ID NO 8.

In some embodiments, the anti-Cx43 antibody used in the method contains a heavy chain having an amino acid sequence that has at least 80%, at least 85%, at least 90%, at least 95%, or at least 99% sequence identity to any one of SEQ ID NOS 9 and 11-13. In some embodiments, the anti-Cx43 antibody contains a light chain having an amino acid sequence that has at least 80%, at least 85%, at least 90%, at least 95%, or at least 99% sequence identity to any one of SEQ ID NO 10. In some embodiments, the anti-Cx43 antibody contains a heavy chain having an amino acid sequence that has at least 80%, at least 85%, at least 90%, at least 95%, or at least 99% sequence identity to SEQ ID NO 9; and a light chain having an amino acid sequence that has at least 80%, at least 85%, at least 90%, at least 95%, or at least 99% sequence identity to any one of SEQ ID NO 10. In some other specific embodiments, the anti-Cx43 antibody contains a heavy chain having an amino acid sequence that has at least 80%, at least 85%, at least 90%, at least 95%, or at least 99% sequence identity to SEQ ID NO 11; and a light chain having an amino acid sequence that has at least 80%, at least 85%, at least 90%, at least 95%, or at least 99% sequence identity to any one of SEQ ID NO 10. In some other specific embodiments, the anti-Cx43 antibody contains a heavy chain having an amino acid sequence that has at least 80%, at least 85%, at least 90%, at least 95%, or at least 99% sequence identity to SEQ ID NO 12; and a light chain having an amino acid sequence that has at least 80%, at least 85%, at least 90%, at least 95%, or at least 99% sequence identity to any one of SEQ ID NO 10. In some yet other specific embodiments, the anti-Cx43 antibody contains a heavy chain having an amino acid sequence that has at least 80%, at least 85%, at least 90%, at least 95%, or at least 99% sequence identity to SEQ ID NO 13; and a light chain having an amino acid sequence that has at least 80%, at least 85%, at least 90%, at least 95%, or at least 99% sequence identity to any one of SEQ ID NO 10.

In some embodiments, the anti-Cx43 antibody contains a heavy chain having the amino acid sequence of any one of SEQ ID NOS 9 and 11-13. In some embodiments, the anti-Cx43 antibody contains a light chain having the amino acid sequence of SEQ ID NO 10. In some embodiments, the anti-Cx43 antibody contains a heavy chain having the amino acid sequence of SEQ ID NO 9, and a light chain having the amino acid sequence of SEQ ID NO 10. In some embodiments, the anti-Cx43 antibody comprises a heavy chain having the amino acid sequence of SEQ ID NO 11, and a light chain having the amino acid sequence of SEQ ID NO 10. In some embodiments, the anti-Cx43 antibody contains a heavy chain having the amino acid sequence of SEQ ID NO 12, and a light chain having the amino acid sequence of SEQ ID NO 10. In some embodiments, the anti-Cx43 antibody contains a heavy chain having the amino acid sequence of SEQ ID NO 13, and a light chain having the amino acid sequence of SEQ ID NO 10.

In some embodiments, the anti-Cx43 antibody can include a mixture, or cocktail, of two or more anti-Cx43 antibodies, each of which binds to the same or different epitope on Cx43.

As “epitope” includes any determinant, preferably a polypeptide determinant, capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three-dimensional structural characteristics, and/or specific charge characteristics. In one embodiment, an epitope is a region of an antigen that is bound by an antibody. In certain embodiments, an antibody is said to specifically bind an antigen when it preferentially recognizes its target antigen in a complex mixture of proteins and/or macromolecules. Methods for epitope mapping are well known in the art, such as X-ray co-crystallography, array-based oligo-peptide scanning, site-directed mutagenesis, high throughput mutagenesis mapping and hydrogen-deuterium exchange.

Epitopes can be formed both from contiguous amino acids or noncontiguous amino acids juxtaposed by tertiary folding of a protein. Epitopes formed from contiguous amimo acid are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

An “antigen” is a molecule capable of being bound by an antibody or T-cell receptor. In certain embodiments, binding moieties other than antibodies are engineered to specifically bind to an antigen, e.g., aptamers, avimers, and the like. In various embodiments, provided herein is an anti-Cx43 antibody that binds an epitope located within, partially or entirely, the amino acid sequence of FLSRPTEKTI (SEQ ID NO 14). In some embodiments, the epitope can include one or more amino acids selected from the group consisting of F1, S3, R4, P5, T6, E7, K8, T9, and 110 of SEQ ID NO 14. In one embodiment, the epitope consists of F1, S3, R4, P5, T6, E7, K8, T9, and 110 of SEQ ID NO 14. In some embodiments, the epitope can include all ten amino acids of SEQ ID NO 14. In certain embodiments, the epitope consists of all ten amino acids of SEQ ID NO 14.

The anti-Cx43 antibody is substantially pure and desirably substantially homogeneous (i.e., free from contaminating proteins etc.). “Substantially pure” antibody means a composition comprising at least about 90% antibody by weight, based on total weight of the protein in the composition, at least about 95% or 97% by weight. “Substantially homogeneous” antibody means a composition comprising protein wherein at least about 99% by weight of protein is specific antibody, e.g., anti-Cx43 antibody, based on total weight of the protein.

In some embodiments, the anti-Cx43 antibody is a humanized antibody. In some embodiments, the anti-Cx43 antibody is a monoclonal antibody. In some a humanized monoclonal antibody.

### IV. Anti-Cx43 Antibody Formulation

One aspect of the present disclosure provides a pharmaceutical formulation that contains an anti-Cx43 antibody for treating cancer, e.g., a bone cancer including osteosarcoma. In some embodiments, the anti-Cx43 antibody promotes opening of Cx43 hemichannels in osteocytes. The anti-Cx43 antibody can be a full antibody or an antigen-binding fragment thereof.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, and by the use of surfactants. In many cases, it may be useful to include isotonic agents, for example, sugars, polyalcohols such as mannitol, sorbitol, or sodium chloride in the composition. Prolonged absorption of the injectable compositions can he brought about by including in the composition,m agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration. Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol phenol sorbic acid, and the like. it may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

It may be advantageous to formulate parenteral compositions of unit dosage form for ease of administration and uniformity of dosage. Unit dosage form as used herein refers to physically discrete units suited as unitary dosages for the patients to be treated: each unit contains a predetermined quantity of active agent calculated to produce the desired therapeutic effect in association with any required pharmaceutical carrier. The specification for unit dosage forms are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitations, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The formulations described herein are administered to a mammal in need of treatment with the anti-Cx43 antibodies, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes.

In some embodiments, the formulations are administered to the mammal by intravenous or subcutaneous (i.e., beneath the skin) administration. For such purposes, the formulation may be injected using a syringe. However, other devices for administration of the formulation are available such as injection devices (e.g., the INJECT-EASE™ and GENJECT™ devices); injector pens (such as the GENPEN™); auto-injector devices, needleless devices (e.g., MEDIJECTOR™ and BIOJECTOR™); and subcutaneous patch delivery systems.

In a specific embodiment, the present disclosure is directed to kits for a single dose-administration unit. Such kits comprise a container of an aqueous formulation of therapeutic protein or antibody, including both single or multi-chambered pre-filled syringes. Exemplary pre-filled syringes are available from Vetter GmbH, Ravensburg, Germany.

### V. Method of Treating Bone Cancer

Another aspect of the present disclosure provides methods of treating a cancer, e.g., bone cancer including osteosarcoma. The methods can involve promoting/enhancing opening of Cx43 hemichannels in osteocytes in a subject in need thereof by using, e.g., an anti-Cx43 antibody. In some embodiments, the subject in need thereof is at least 2 years old. In some embodiments, the subject in need thereof is at least 12 years old.

Currently there is a lack of second-line therapy for osteosarcoma, therefore, Cx43 modulation (e.g., promoting the opening of Cx43 hemichannel using an anti-Cx43 antibody) can be used as a second line therapy for those who failed standard care of osteosarcoma, or at least one line of therapy for treating osteosarcoma. The Cx43 modulation can be a stand-alone therapy for bone cancer including osteosarcoma, or in combination with other cancer therapies described herein or known in the art.

In some embodiments, the anti-Cx43 antibody is used as a second line therapy in a subject who failed at least one line of therapy (standard care) of a bone cancer (e.g., osteosarcoma). In some other embodiments, the anti-Cx43 antibody is used as a first line therapy in a subject for treatment of bone cancer (e.g., osteosarcoma). In some embodiments, the anti-Cx43 antibody is used as a neoadjuvant treatment of bone cancer (e.g., osteosarcoma) in a subject.

Various cells are able to communicate with each other and with the extracellular environment through hemichannels and gap junctions formed by the protein connexin. Connexin proteins are ubiquitously expressed throughout the body. Six connexin proteins make up one hemichannel, and two hemichannels make up one gap junction channel. Gap junctions are a cluster of channels that are located in the plasma membrane between adjoining cells and they mediate intercellular communication. Hemichannels are a separate entity from gap junction channels. Hemichannels permit the exchange of molecules between the intracellular compartments and the extracellular environment.

Normal bone is made up of three major cell types: bone-forming osteoblasts, bone resorbing osteoclasts, and osteocytes. Osteocytes make up approximately 95% of bone cells and maintain the bone remodeling process by coordinating osteolytic and osteoblastic activities. When cancer cells invade the bone, many of the normal bone functions are affected. Cancer cells interact with the local microenvironment to promote cancer cell survival via bone destruction and vascularization.

Osteocytes express hemichannels known as connexin (Cx) 43 hemichannels. These osteocyte hemichannels are normally closed and can be opened when exposed to mechano-stimulation, which leads to the release of various factors into the bone microenvironment. The factors released by hemichannel opening can mediate other processes that can decrease tumor cell migration and bone metastasis. Alendronate (AD), an efficacious and commonly used bisphosphonate drug has been shown to be able to open Cx43 hemichannels in osteocytes.

Bisphosphonates are a class of drugs known for treating many bone disorders including bone metastasis. It has been shown that administration of bisphosphonates to be associated with a decrease in the incidence of bone metastasis and a decrease in death rate in patients with breast cancer. AD has been associated with decreased tumor growth as well as reduced bone destruction and pain. AD inhibits osteoclast activity and induces the opening of Cx43 hemichannels in osteocytes. However, AD administration is accompanied by multiple, severe side-effects.

Cx43 is also known as gap junction alpha-1 protein (GJA1), which is a 43.0 kDa protein composed of 382 amino acids (NCBI Reference Sequence: NP 000156.1). GJA1 contains a long C-terminal tail, an N-terminal domain, and multiple transmembrane domains. The protein passes through the phospholipid bilayer four times, leaving its C- and N-terminals exposed to the cytoplasm. The C-terminal tail is composed of 50 amino acids and includes post-translational modification sites, as well as binding sites for transcription factors, cytoskeleton elements, and other proteins. As a result, the C-terminal tail is central to functions such as regulating pH gating and channel assembly. Notably, the DNA region of the GJA1 gene (NCBI Gene ID: 2697) encoding this tail is highly conserved, indicating that it is either resistant to mutations or becomes lethal when mutated. Meanwhile, the N terminal domain is involved in channel gating and oligomerization and, thus, may control the switch between the channel's open and closed states. The transmembrane domains form the gap junction channel while the extracellular loops facilitate proper channel docking. Moreover, two extracellular loops form disulfide bonds that interact with two hexamers to form a complete gap junction channel.

“Treatment” refers to therapeutic treatment. Those in need of treatment include those already with disease. Hence, the patient, e.g., human, to be treated herein may have been diagnosed as suffering from a disease, such as bone cancer. A disease, e.g., bone cancer, is “inhibited” or “treated” if at least one symptom (as determined by responsiveness/non-responsiveness, or indicators known in the art and described herein) of the condition is alleviated, terminated, slowed, minimized, or prevented. The terms “patient” and “subject” are used interchangeably herein.

The term “subject” or “patient” refers to either a human or non-human, such as primates, mammals, and vertebrates. In particular embodiments, the subject is a human.

Treatment can be suitably administered to subjects, particularly humans, suffering from, having, susceptible to, or at risk of developing such cancer, e.g., bone cancer including osteosarcoma. Determination of those subjects “at risk” can be made by any objective or subjective determination by a diagnostic test or opinion of a subject or health care provider (e.g., genetic test, enzyme or protein marker, family history, and the like). Identifying a subject in need of such treatment can be in the judgment of a subject or a health care professional and can be subjective (e.g., opinion) or objective (e.g., measurable by a test or diagnostic method).

The anti-Cx43 antibody can be stored as a lyophilized solid or an aqueous formulation, or any other forms known in the art. In the case of an anti-Cx43 antibody which is stored as a lyophilized solid, the antibody is reconstituted in a solution such as water for injection prior to administration. If prepared for infusion either from a lyophilized form or an aqueous formulation, the final concentration, e.g., after dilution of the reconstituted antibody (e.g., in a saline, Ringer's or 5% dextrose infusion system) of the anti-Cx43 antibody can be about 0.1 mg/ml to about 80 mg/ml for administration. The final concentration may be about 0.1 mg/ml to about 80 mg/ml, about 0.5 mg/ml to about 70 mg/ml, about 1 mg/ml to about 60 mg/ml, about 5 mg/ml to about 50 mg/ml, about 10 mg/ml to about 40 mg/ml, about 15 mg/ml to about 30 mg/ml, or about 20 mg/ml to about 25 mg/ml. In some embodiments, the final dosage form may be at a concentration of less than 0.1 mg/ml, about 0.1 mg/ml, about 0.5 mg/ml, about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml or higher than 80 mg/ml.

For example, an effective amount or a dose of the anti-Cx43 antibody ranges from about 0.001 mg/kg to about 300 mg/kg. In some embodiments, a dose of the anti-Cx43 antibody is about 0.01 mg/kg to about 200 mg/kg, about 0.1 mg/kg to about 100 mg/kg, about 0.2 mg/kg to about 90 mg/kg, about 0.5 mg/kg to about 80 mg/kg, about 0.8 mg/kg to about 70 mg/kg, about 1 mg/kg to about 60 mg/kg, about 3 mg/kg to about 50 mg/kg, about 6 mg/kg to about 40 mg/kg, about 9 mg/kg to about 36 mg/kg, about 12 mg/kg to about 33 mg/kg, about 15 mg/kg to about 30 mg/kg, about 18 mg/kg to about 27 mg/kg, or about 21 mg/kg to about 24 mg/kg. In some embodiments, a dose of the anti-Cx43 antibody is about 0.001 mg/kg, about 0.01 mg/kg, about 0.1 mg/kg, about 1 mg/kg, about 3 mg/kg, about 6 mg/kg, about 9 mg/kg, about 12 mg/kg, about 15 mg/kg, about 18 mg/kg, about 21 mg/kg, about 24 mg/kg, about 27 mg/kg, about 30 mg/kg, about 33 mg/kg, about 36 mg/kg, about 40 mg/kg, about 50 mg/kg, about 60 mg/kg, about 70 mg/kg, about 80 mg/kg, about 90 mg/kg, about 100 mg/kg, about 120 mg/kg, about 140 mg/kg, about 160 mg/kg, about 180 mg/kg, about 200 mg/kg, about 220 mg/kg, about 240 mg/kg, about 260 mg/kg, about 280 mg/kg, about 300 mg/kg, or more than 300 mg/kg.

In some specific embodiments, the effect amount or a dose of the anti-Cx43 antibody is 1 mg/kg, 3 mg/kg, 6 mg/kg, 9 mg/kg, 12 mg/kg, 15 mg/kg, 18 mg/kg, 21 mg/kg, 24 mg/kg, 27 mg/kg, 30 mg/kg, 33 mg/kg, or 36 mg/kg.

A therapeutically effective amount will vary depending upon the patient and disease condition being treated, the weight and age of the patient, the severity of the disease condition, the manner of administration, course of the condition, patient's clinical history and response to anti-Cx43 antibody and the like, which can readily be determined by one of ordinary skill in the art. In some embodiments, an effective amount or a dose of the anti-Cx43 antibody can range from about 1 mg to about 25,000 mg. In some embodiments, a dose of the anti-Cx43 antibody is about 2 mg to 20,000 mg, about 5 mg to 15,000 mg, about 10 mg to 10,000 mg, about 15 mg to 10,000 mg, about 20 mg to 5000 mg, about 25 mg to 4000 mg, about 30 mg to 3000 mg, about 40 mg to 2000 mg, about 50 mg to 1000 mg, about 60 mg to 900 mg, about 70 mg to 800 mg, about 80 mg to 700 mg, about 90 mg to 600 mg, about 100 mg to 500 mg, about 150 mg to 450 mg, about 200 mg to 400 mg, about 250 mg to 350 mg, or about 275 mg to 325 mg. In some embodiments, a dose of the anti-Cx43 antibody is less than 1 mg, about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 75 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1250 mg, about 1500 mg, about 1750 mg, about 2000 mg, about 2250 mg, about 2500 mg, about 2750 mg, about 3000 mg, about 4000 mg, about 5000 mg, about 6000 mg, about 7000 mg, about 8000 mg, about 9000 mg, about 10,000 mg, about 12,500 mg, about 15,000 mg, about 17,500 mg, about 20,000 mg, about 22,500 mg, about 25,000 mg, or more than 25,000 mg.

In the methods as described herein, an effective amount or a dose of the anti-Cx43 antibody is administered about once every day, about once every 2 days, about once every 3 days, about once every 4 days, about once every 5 days, about once every 6 days, about once every week, about once every 8 days, about once every 9 days, about once every 10 days, about once every 11 days, about once every 12 days, about once every 13 days, about once every 2 weeks, about once every 15 days, about once every 16 days, about once every 17 days, about once every 18 days, about once every 19 days, about once every 20 days, about once every 3 weeks, about once every 22 days, about once every 23 days, about once every 24 days, about once every 25 days, about once every 26 days, about once every 27 days, about once every 4 weeks, about once every 29 days, about once every 30 days, about once every 31 days, about once every 32 days, about once every 33 days, about once every 34 days, about once every 5 weeks, about once every 36 days, about once every 37 days, about once every 38 days, about once every 39 days, about once every 40 days, or about once every 41 days, about once every 6 weeks, about once every 7 weeks, about once every 8 weeks, about once every 9 weeks, about once every 10 weeks, about once every 11 weeks, about once every 12 weeks, about once every 13 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks or 6 months, or about once every more than 24 weeks or 6 months.

In some specific embodiments, a dose of the anti-Cx43 antibody is administered about once every 3 weeks. In one particular embodiment, a dose of the anti-Cx43 antibody is administered once every 3 weeks.

According to certain embodiments of the present disclosure, multiple doses of an anti-Cx43 antibody (or a pharmaceutical composition comprising a combination of an anti-Cx43 antibody and any of the additional therapeutically active agents mentioned herein) may be administered to a subject over a defined time course. The methods according to this aspect of the disclosure comprises sequentially administering to a subject multiple doses of an anti-Cx43 antibody of the disclosure.

The anti-Cx43 antibody is suitably administered to the patient at one time or over a series of treatments and may be administered to the patient at any time from diagnosis onwards. The anti-Cx43 antibody may be administered as the sole treatment or in conjunction with other drugs or therapies useful in treating the condition in question.

In the methods as described herein, a dose of the anti-Cx43 antibody can be administered to a patient over less than 5 minutes, about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more than 180 minutes. In some specific embodiments, a dose of the anti-Cx43 antibody can be administered to a patient over about 30 minutes. In one specific embodiment, a dose of the anti-Cx43 antibody can be intravenously administered to a patient over 30 minutes.

In the methods described herein, an anti-Cx43 antibody is administered to a patient. If the anti-Cx43 antibody is in a formulation which is in a solid, e.g., dry state, the process of administration can comprise a step of converting the formulation to a liquid state. In one aspect, a dry formulation can be reconstituted, e.g., by a liquid as described above, for use in injection, e.g. intravenous, intradermal, intramuscular, intratumoral, intraperitoneal or subcutaneous injection. In one specific embodiment, the In another aspect, a solid or dry formulation can be administered topically, e.g., in a patch, cream, aerosol or suppository.

In the methods described herein, the anti-Cx43 antibody may be administered to a subject alone or in conjunction with another agent. The anti-Cx43 antibody can be administered before, along with or subsequent to administration of the additional agent. In one embodiment, the dose of the co-administered agent can be decreased over time or completely tapered during the period of treatment by the anti-Cx43 antibody. Non-limiting examples the co-administered agent include chemotherapeutic drugs, radiotherapy, immune therapy and surgery.

Non-limiting examples of such chemotherapeutic drugs include platinum-based chemotherapy drugs (e.g., cisplatin, carboplatin), taxanes (e.g., paclitaxel (Taxol®), docetaxel (Taxotere®), EndoTAG-PM (a formulation of paclitaxel encapsulated in positively charged lipid-based complexes; MediGene), Abraxane® (a formulation of paclitaxel bound to albumin)), tyrosine kinase inhibitors (e.g., imatinib/Gleevec®, sunitinib/Sutent®, dasatinib/Sprycel®), and combinations thereof.

In another embodiment, the at least one additional anti-cancer agent comprises an EGFR inhibitor, such as an anti-EGFR antibody or a small molecule inhibitor of EGFR signaling. An exemplary anti-EGFR antibody is cetuximab (Erbituxl®) Cetuximab is commercially available from ImClone Systems Incorporated. Other examples of anti-EGFR antibodies include matuzumab (EMD72000), panitumumab (Vectibix®; Amgen); nimotuzumab (TheraCIM™) and mAb 806. An exemplary small molecule inhibitor of the EGFR signaling pathway is gefitinib (Iressa@), which is commercially available from AstraZeneca and Teva. Other examples of small molecule inhibitors of the EGFR signaling pathway include erlotinib HCL (OSI-774: Tarceva®), OSI Pharma); lapatinib (Tykerb®, GlaxoSmithKline); canertinib (canertinib dihydrochloride, Pfizer): pelitinib (Pfizer): PKI-166 (Novartis); PD158780; and AG 1478 (4-(3-Chloroanillino)-6,7-dimethoxyquinazoline).

In yet another embodiment, the at least one additional anti-cancer agent comprises a VEGF inhibitor. An exemplary VEGF inhibitor comprises an anti-VEGF antibody, such as bevacizumab (Avastatin@: Genentech). In still another embodiment, the at least one additional anti-cancer agent comprises an anti-ErbB2 antibody. Suitable anti-ErbB2 antibodies include trastuzumab and pertuzumab.

In one aspect, the improved effectiveness of a combination according to the disclosure can be demonstrated by achieving therapeutic synergy. The term “therapeutic synergy” or “synergistic effect” is used when the combination of two products at given doses is more efficacious than the best of each of the two products alone at the same doses. In one example, therapeutic synergy can be evaluated by comparing a combination to the best single agent using estimates obtained from a two-way analysis of variance with repeated measurements (e.g., time factor) on parameter tumor volume.

In some embodiments, the patient or subject previously failed at least one line of therapy or standard care of a bone cancer (e.g., osteosarcoma). Non-limiting examples of the failed therapies or standard care of a bone cancer (e.g., osteosarcoma) is any therapy or agent described elsewhere in the present disclosure which can be co-administered with the anti-Cx43 antibodies.

In some embodiments, the patient or subject previously failed at least one therapy for osteosarcoma. In some specific embodiments, the at least one therapy for osteosarcoma comprising chemotherapy, surgery, radiotherapy, or immune therapy, or the combination thereof. In some specific embodiments, the patient or subject previously failed at least one therapy comprising methotrexate, doxorubicin, cisplatin, and ifosfamide, apatinib, anlotinib, vindesine, vincristine, docetaxel, paclitaxel, irinotecan, bortezomib, albumin-bound paclitaxel, nedaplatin (Aqupla), pemetrexed, etoposide, gemcitabine, lobaplatin, recombinant human endostatin, eribulin, dacarbazine, pazopanib, immune checkpoint inhibitors, surgery, or radiotherapy, or the combination thereof.

The patient or subject, before, during, or after being treated with the anti-Cx43 antibody, is examined for cancer related parameters using technologies and methods known in the art. Non-limiting examples of the common medical technologies and methods used to examine and diagnose osteosarcoma include: X-rays, computed tomography (CT) scan, magnetic resonance imaging (MRI), positron emission tomography (PET), bone scintigraphy (Bone Scan), biopsy, EQ-5D questionnaire for assessing quality of life, PERCIST criteria for assessing disease progression, numeric rating scale for assessing pain degree. The choice of technologies and methods, and the frequency of examination can be determined and/or adjusted by a person skilled in the art based on the patient's or subject's specific conditions.

In some embodiments, treatment with the anti-Cx43 antibody for osteosarcoma improves disease progression as assessed by ECOG physical performance criteria, Numeric Rating Scale (NRS) Score, EQ-5D, PERCIST criteria, RECIST criteria, ICDS criteria, EORTC evaluation criteria, and/or PERCIST, as comparing with a control, which can include but is not limited to an osteosarcoma population who receive or failed the same lines of therapy as the subject, but do not receive the anti-Cx43 antibody for osteosarcoma.

In some embodiments, treatment with the anti-Cx43 antibody for osteosarcoma improves overall survival by 1 month, at least 2 months, at least 3 months, at least 4 months, at least 5 months, or at least 6 months comparing with a control value, which can include but is not limited to the mean or median of overall survival of an osteosarcoma population who receive or failed the same lines of therapy as the subject, but do not receive the anti-Cx43 antibody for osteosarcoma.

In some embodiments, treatment with the anti-Cx43 antibody for osteosarcoma improves 6-month progression-free survival by 1 month, at least 2 months, at least 3 months, at least 4 months, at least 5 months, or at least 6 months comparing with a control value, which can include but is not limited to the mean or median of overall survival of an osteosarcoma population who receive or failed the same lines of therapy as the subject, but do not receive the anti-Cx43 antibody for osteosarcoma.

In some embodiments, the subject treated with the anti-Cx43 antibody for osteosarcoma has a duration of response of at least 10 days, at least 15 days, at least 20 days, at least 25 days, at least 1 month, at least 2 months, at least 3 months, at least 4 months, at least 5 months, at least 6 months, or more.

In some embodiments, the subject treated with the anti-Cx43 antibody for osteosarcoma has a time to response less than 2 years, less than 11 months, less than 10 months, less than 9 months, less than 8 months, less than 7 months, less than 6 months, less than 5 months, less than 4 months, less than 3 months, less than 2 months, less than 1 months, less than 25 days, less than 20 days, less than 15 days, less than 10 days, or less than 5 days.

In some embodiments, treatment with the anti-Cx43 antibody for osteosarcoma reduces the tumor size by at least 1%, at least 2%, at least 3%, at least 5%, at least 8%, at least 10%, at least 15%, at least 20%, or at least 30%.

In some embodiments, treatment with the anti-Cx43 antibody for osteosarcoma improves the Europe Health-related Quality of Life Questionnaire 5 Dimensions (EQ-5D) score. In some embodiments, treatment with the anti-Cx43 antibody for osteosarcoma improves the EQ-5D health index to at least 0.1, at least 0.2, at least 0.3, at least 0.4, at least 0.5, at least 0.6, at least 0.7, at least 0.8, at least 0.9, or 1, compared with baseline. In some embodiments, treatment with the anti-Cx43 antibody for osteosarcoma improves the visual analog scale (VAS) score of the EQ-5D questionnaire by at least 1, at least 2, at least 3, at least 4, at least 5, at least 10, at least 20, at least 30, at least 40, at least 50, at least 60, at least 70, at least 80, at least 90, or 100.

In some embodiments, treatment with the anti-Cx43 antibody for osteosarcoma reduces Numeric Rating Scale (NRS) score by at least 1, at least 2, at least 3, at least 4, at least 5, at least 6, at least 7, at least 8, at least 9, or 10.

In some embodiments, treatment with the anti-Cx43 antibody for osteosarcoma improves bone mineral density (BMD) of the lumbar spine, hip, and femur and neck. In some embodiments, treatment with the anti-Cx43 antibody for osteosarcoma improves bone mineral density (BMD) of the lumbar spine, hip, and femur and neck by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more.

Treatment with the anti-Cx43 antibody for osteosarcoma reduces the dose and/or frequency of morphine use by the subject or patient. In some embodiment, treatment with the anti-Cx43 antibody for osteosarcoma reduces the dose and/or frequency of morphine use by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In certain embodiments, treatment with the anti-Cx43 antibody for osteosarcoma completely tapers the use of morphine use.

A “response” or “being responsive” to a treatment refers to the subject having improvement of at least one parameter of disease progression. The subject can have “partial response” or “complete response” to a treatment. The response, partial response or complete response to a treatment can be determined based on methods known in the art. A person skilled in the art can determine the proper methods based on the type of diseases being evaluated. Non-limiting examples of the methods include RECIST criteria, ICDS criteria and PERCIST criteria. In some embodiments, a subject has complete response to a treatment of the anti-Cx43 antibody. In some other embodiments, the subject has a partial response to a treatment of the anti-Cx43 antibody.

“Adverse Drug Reaction” or “ADR” is any reaction harmful to human body or unintended reaction which is considered to possibly be related to the drug in a clinical study. The drug and the AE are at least in one possibly reasonable causality, i.e., the correlation cannot be ruled out.

“Serious Adverse Event” or “SAE” is one of the following situations after the subject receives the drug: (i) resulting in death; (ii) Life-threatening: refers to the subject's risk of death at the time of the event, not to the risk of death if the event deteriorates; (3) requiring hospitalization or prolongation of hospitalization. It is understood by a person in the art that SAE includes but is not limited to the foregoing list. Non-limiting examples of “dose limiting toxicity” include Grade 4 neutropenia lasting for ≥7 days; or Grade ≥3 neutropenia with fever (ANC <1000/mm³ with single temperature >38.3° C. or persistent temperature ≥38° C. for more than 1 h) or with infection; Grade 4 thrombocytopenia or Grade 3 thrombocytopenia with hemorrhage; Grade ≥3 hepatotoxicity: ALT or AST ≥5× upper limit of normal (ULN) (for subjects with normal baseline) or ≥5× baseline value (for subjects with combined liver metastasis and abnormal baseline); or bilirubin ≥3×ULN (for subjects with normal baseline value) or ≥3× baseline value (for subjects with abnormal baseline value); Grade ≥3 nephrotoxicity: Serum creatinine ≥3×ULN (for subjects with normal baseline value) or ≥3× baseline value (for subjects with abnormal baseline value); Grade ≥3 cardiotoxicity: QTc ≥500 ms or QTc increase ≥60 ms.

“Treatment Emergent Adverse Event” or “TEAE” refers to the event that occurs during the treatment but does not occur or deteriorates relative to the pre-treatment period.

In some embodiments, after treatment with the anti-Cx43 antibody, the subject does not experience: (i) adverse event, serious adverse event or grade 3 or higher adverse events; (ii) treatment emergent adverse event (TEAE); (iii) adverse drug reaction (ADR); (iv) Cx43-related cardiac events or severe hepatic events; and/or (v) dose limiting toxicity.

The subject or patient treated by the method described herein has an age of at least 2 years old. In some embodiments, the subject or patient is at least 2, at least 3, at least 4, at least 5, at least 6, at least 7, at least 8, at least 9, at least 10, at least 11, at least 12, at least 13, at least 14, at least 15, at least 16, or at least 17 years old. In some embodiments, the subject or patient is at least 12 years old. In some embodiments, the subject or patient is at least 18 years old. In some embodiments, the subject or patient is a pediatric subject or patient. In some embodiments, the subject or patient is an adult.

### EXAMPLES

The following examples are included to demonstrate preferred embodiments of the invention. It should be appreciated by those of skill in the art that the techniques disclosed in the examples which follow—represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Juvenile Mouse and Preclinical Study of ALMB-0168

### Objectives

ALMB-0168 was developed by AlaMab Therapeutics Inc. for clinical treatment of cancer with bone metastases. The objectives of this study were to evaluate the potential toxicity (including developmental toxicity), toxic target organs, and toxicokinetic profile of ALMB-0168 following intravenous injections to juvenile C57BL/6J mice once weekly for 7 consecutive weeks (8 doses in total), and to evaluate the reversibility of toxicity following a 4-week recovery period.

### Method

A total of 928 juvenile C57BL/6J mice (464 male mice and 464 female mice, age on the first dosing day: 21 to 23 days old) were randomly assigned to Groups 1 to 8 with 40 mice/sex/group in Groups 1 to 4 as main groups for toxicity study, and with 40 mice/sex in Group 5 and 88 mice/sex/group in Groups 6 to 8 as satellite groups for toxicokinetic (TK) and anti-drug antibody (ADA) analysis. Mice in Groups 1 and 5 were dosed with Sodium Chloride Injection as negative control groups; mice in Groups 2 and 6, 3 and 7, and 4 and 8 were dosed with ALMB-0168 at 25 mg/kg (low dose), 75 mg/kg (middle dose) or 250 mg/kg (high dose), respectively. Mice were dosed by intravenous injection via caudal vein once weekly for 7 consecutive weeks (8 doses in total) followed by a 4-week recovery period. The dose volume was 20 mL/kg and dosing procedure was finished within 15 seconds for each animal. The first 20 animals/sex/group in Groups 1 to 4 were euthanized after 7 weeks of dosing (Day 51), and remaining 20 animals/sex/group were euthanized after the 4-week recovery period (Day 78).

Parameters evaluated in this study included clinical observations, body weight, food consumption, body temperature, ophthalmologic examinations, physical development examinations (tibia length, body length, tail length, physical development (testicular descent and preputial separation in males, and vaginal opening in females)), behavioral development examinations (locomotor activity test and learning and memory abilities), hematology, coagulation, clinical chemistry, lymphocyte subset, cytokines, serum anti-drug antibodies (ADA), sperm parameters (sperm counts, motility, and sperm morphology) and estrous cycle.

Serum concentration of ALMB-0168 and toxicokinetic parameters were analyzed following dosing on Day 1 and Day 50. Right femurs from animals (5 mice/sex/group) in Groups 1 to 4 were collected for bone mineral density (BMD) and bone mineral content (BMC) tests. Animals in Groups 1 to 4 (10/sex/group at the end of dosing period, 5/sex/group at the end of recovery period) were subjected to a necropsy for organ weight and histopathological evaluation for animals in Groups 1 and 4 and abnormal tissues in Groups 2 and 3.

### Results

Samples of ALMB-0168 formulations at 12.5, 3.75, and 1.25 mg/mL were analyzed for concentration and homogeneity after preparation on Day 1, Day 22, and Day 50. Actual concentrations of the test article formulations ranged from 95.67% to 106.73% of the nominal concentrations, and the coefficient of variation (CV %) of the upper, middle, and lower layers of each formulation ranged from 0.27% to 1.36%, indicating that the formulations were properly prepared and acceptable for use in this study.

During the study, no mortality or moribundity was noted in any group.

During the study, defect in right eye tissue in one female of the negative control group, sparse hair in two females of the 25 mg/kg group, hunched back and defect in left eye tissue in one female, respectively of the 250 mg/kg group were noted. These changes were not considered test article-related as they occurred in low incidences and had no dose-dependence, or they were also noted in the negative control group.

During the study, no test article-related changes in body weight, body weight gain, and growth velocity per unit time were noted in any animals of the 25, 75, and 250 mg/kg groups. Compared to the concurrent negative control group of the same sex, statistically significant changes in body weight, body weight gain and growth velocity per unit time were noted in animals of the 25, 75 and 250 mg/kg groups at some time points. However, these changes were not considered test article-related since they were slight and had no dose-dependence or regularity, and body weight gain of the animals was normal. Body weights and weight gains of animals in each dose group are shown in Table 1 below.

TABLE 1
Summary of Body Weight and Body Weight Gain.

| | Dose Level (mg/kg) | | | | | | | |
|---|---|---|---|---|---|---|---|---|
| Parameters | 0 | 25 | 75 | 250 | 0 | 25 | 75 | 250 |
| (% change^{a}) | M | M | M | M | F | F | F | F |

Body Weight/(g)

| | | | | | | | | |
|---|---|---|---|---|---|---|---|---|
| D 28 | 22.67 | 22.26 | 22.32 | 21.65* | 17.96 | 17.93 | 17.53 | 17.63 |
| | | | | 4.5%↓ | | | | |
| D 35 | 24.33 | 23.08 | 23.92 | 23.19* | 18.82 | 19.07 | 18.38 | 18.26 |
| | | | | 4.7%↓ | | | | |
| D 42 | 24.74 | 24.30 | 24.20 | 23.74* | 19.40 | 19.31 | 18.71* | 18.65* |
| | | | | 4.0%↓ | | | 3.6%↓ | 3.9%↓ |
| D 56 | 25.99 | 25.62 | 25.14 | 24.78* | 19.86 | 20.10 | 19.51 | 19.73 |
| | | | | 4.7%↓ | | | | |

Body Weight Gain/(g)

| | | | | | | | | |
|---|---|---|---|---|---|---|---|---|
| W 2 | 5.84 | 5.48 | 5.67 | 5.17* | 3.96 | 4.15 | 3.82 | 3.71 |
| | | | | 11.4%↓ | | | | |
| W 6 | 0.40 | 0.50 | 0.27 | 0.56 | 0.59 | 0.24* | 0.34* | 0.39 |
| | | | | | | 59.6%↓ | 42.6%↓ | |
| W 7 | 0.57 | 0.70 | 0.68 | 0.91* | 0.32 | 0.61* | 0.76* | 0.82* |
| | | | | 60.4%↑ | | 89.8%↑ | 135.9%↑ | 157.0%↑ |
| W 8 | 0.45 | 0.48 | 0.13* | 0.27 | 0.31 | 0.11 | 0.02 | −0.03 |
| | | | 71.1%↓ | | | | | |

Notes:
M indicates male;
F indicates female;
↑indicates increase,
↓indicates decrease.
^{a}indicates percent difference from the negative control group;
*indicates p ≤ 0.05 as compared with the concurrent negative control group of the same sex.

During the study, no test article-related changes in food consumption were noted in animals of the 25, 75, and 250 mg/kg groups. Compared to the concurrent negative control group of the same sex, statistically significant changes in food consumption were noted in animals of the 25, 75 and 250 mg/kg groups at some time points, but these changes were not considered test article-related since they were slight, and no obvious changes were noted in body weight at the same time points. Food consumption of animals in each dose group are shown in the Table 2 below.

TABLE 2
Summary of Food Consumption

| | Dose Level (mg/kg) | | | | | | | |
|---|---|---|---|---|---|---|---|---|
| Parameters | 0 | 25 | 75 | 250 | 0 | 25 | 75 | 250 |
| (% change^{a}) | M | M | M | M | F | F | F | F |

Food Consumption/(g)

| | | | | | | | | |
|---|---|---|---|---|---|---|---|---|
| W 1 | 2.34 | 2.32 | 2.24* | 2.29 | 2.20 | 2.22 | 2.18 | 2.17 |
| | | | 4.0%↓ | | | | | |
| W 2 | 3.16 | 3.06* | 3.06* | 3.04* | 2.82 | 2.78 | 2.74* | 2.80 |
| | | 3.0%↓ | 3.2%↓ | 3.8%↓ | | | 2.6%↓ | |
| W 3 | 3.47 | 3.47 | 3.40* | 3.34* | 3.04 | 2.97* | 2.93* | 3.01 |
| | | | 2.2%↓ | 3.8%↓ | | 2.1%↓ | 3.5%↓ | |
| W 4 | 3.39 | 3.32 | 3.33 | 3.30 | 2.82 | 2.79 | 2.74* | 2.80 |
| | | | | | | | 2.6%↓ | |
| W 5 | 3.39 | 3.32* | 3.44 | 3.43 | 2.91 | 2.89* | 2.85* | 2.86* |
| | | 2.2%↓ | | | | 0.7%↓ | 1.9%↓ | 1.8%↓ |
| W 6 | 3.31 | 3.30 | 3.36 | 3.29 | 2.96 | 2.89* | 2.83* | 2.91* |
| | | | | | | 2.2%↓ | 4.5%↓ | 1.8%↓ |
| W 7 | 3.51 | 3.51 | 3.53 | 3.55 | 3.19 | 3.12* | 3.18 | 3.15 |
| | | | | | | 2.1%↓ | | |
| W 8 | 3.39 | 3.62* | 3.56* | 3.41 | 3.23 | 3.14* | 3.06* | 3.05* |
| | | 6.5%↑ | 4.8%↑ | | | 2.6%↓ | 5.2%↓ | 5.4%↓ |
| W 9 | 3.68 | 3.66 | 3.57 | 3.59 | 3.42 | 3.24* | 3.20* | 3.26* |
| | | | | | | 5.2%↓ | 6.6%↓ | 4.7%↓ |
| W 10 | 3.58 | 3.67* | 3.47 | 3.59 | 3.40 | 3.23* | 3.20* | 3.24* |
| | | 2.5%↑ | | | | 5.2%↓ | 6.0%↓ | 4.9%↓ |
| W 11 | 3.76 | 3.86 | 3.73 | 3.74 | 3.57 | 3.37* | 3.35* | 3.41* |
| | | | | | | 5.5%↓ | 6.0%↓ | 4.4%↓ |

Notes:
M indicates male;
F indicates female;
↑indicates increase,
↓indicates decrease.
^{a}indicates percent difference from the negative control group;
*indicates p ≤ 0.05 as compared with the concurrent negative control group in the same sex.

During the study, no test article-related changes in body temperature were noted in animals of the 25, 75, and 250 mg/kg groups. Compared to the concurrent negative control group of the same sex, statistically significant increases in body temperature were noted in females of all test article groups on Day 50 (body temperature of the test article groups were 36.76° C., 36.86° C. and 37.14° C., respectively, and body temperature of the control group was 36.22° C.). These changes were slight and body temperature values of these animals still fluctuated within normal range of mice. Therefore, they were not considered related to the test article.

During the study, no test article-related abnormal changes in ophthalmologic examinations were noted in animals of the 25, 75, and 250 mg/kg groups. Abnormal pupil, lens opacity, corneal opacity, tissue defect, absence of pupillary light reflex, mydriasis, irregular pupil shape, and intraocular structure not seen were noted in animals of Groups 1 to 4 on Day 49 and Day 76 with incidences of 8/80, 7/80, 12/80 and 8/80. These changes were not considered test article-related as no apparent differences in incidence were noted among the groups, and there was a certain incidence of ophthalmologic abnormalities in this species.

During the study, no test article-related abnormal changes in tibia length were noted in animals of the 25, 75, and 250 mg/kg groups. Compared to the concurrent negative control group of the same sex, statistically significant decreases in tibia length were noted in females of the 75 mg/kg group on Day 14 (decreased by 4.0%), in males of the 250 mg/kg group on Day 14 (decreased by 4.5%), and in females of the 250 mg/kg group on Day 7 and Day 14 (decreased by 7.2% and 4.7%, respectively). These changes were slight and irregular, and therefore they were not considered related to the test article. In addition, no test article-related changes were noted in body length and tail length in animals of the 25, 75, and 250 mg/kg groups. Compared to the concurrent negative control group of the same sex, a statistically significant increase in body length was observed in males of the 250 mg/kg group on Day-1 (increased by 9.5%), but it was not considered test article-related since it was only noted prior to the first dosing.

Compared to the concurrent negative control group of the same sex, no statistically significant or test article-related changes were noted in number of days to reach the physical development criterion (testicular descent, vaginal opening and preputial separation) in animals of the 25, 75, and 250 mg/kg groups during the study. The results of physical development examinations are summarized in Table 3 below.

TABLE 3
Summary of Physical Development Examinations.

| | | Negative | | | |
|---|---|---|---|---|---|
| | | Control | Low (25 | Middle (75 | High (250 |
| | | (0 mg/kg) | mg/kg) | mg/kg) | mg/kg) |
| Testicular | Mean | 7.2 | 7.0 | 7.1 | 7.6 |
| Descent | SD | 0.4 | 0.0 | 0.3 | 0.7 |
| | n | 10 | 10 | 10 | 10 |
| Preputial | Mean | 20.5 | 20.7 | 20.6 | 20.8 |
| Separation | SD | 0.7 | 0.9 | 0.7 | 1.5 |
| | n | 10 | 10 | 10 | 10 |
| Vaginal | Mean | 13.4 | 13.9 | 14.2 | 13.7 |
| Opening | SD | 0.8 | 1.2 | 1.1 | 1.3 |
| | n | 10 | 10 | 10 | 10 |

Note:
“n” indicates the number of animals.

Compared to the concurrent negative control group of the same sex, no statistically significant or test article-related changes were noted in locomotor activity parameters (total path of motion, average speed, resting time, and motion time) in animals of the 25, 75, and 250 mg/kg groups during the study. Results of locomotor activity test are summarized in Tables 4 and 5 below.

TABLE 4
Summary of Locomotor Activity Test - Males.

| | Dose Level | | Total Path of | Average Speed | Resting | Motion |
|---|---|---|---|---|---|---|
| Group | (mg/kg) | | Motion (mm) | (mm/s) | Time (s) | Time (s) |
| Negative | 0 | Mean | 17457.70 | 29.10 | 342.96 | 257.05 |
| Control | | SD | 4001.09 | 6.67 | 43.33 | 43.33 |
| | | n | 10 | 10 | 10 | 10 |
| Low | 25 | Mean | 18379.04 | 30.63 | 330.19 | 269.82 |
| | | SD | 3932.98 | 6.56 | 43.80 | 43.77 |
| | | n | 10 | 10 | 10 | 10 |
| Middle | 75 | Mean | 17739.42 | 29.56 | 339.52 | 260.49 |
| | | SD | 2750.06 | 4.58 | 32.66 | 32.67 |
| | | n | 10 | 10 | 10 | 10 |
| High | 250 | Mean | 17851.76 | 29.75 | 335.48 | 264.53 |
| | | SD | 2897.71 | 4.83 | 32.43 | 32.41 |
| | | n | 10 | 10 | 10 | 10 |

Note:
“n” indicates the number of animals.

TABLE 5
Summary of Locomotor Activity Test - Females.

| | Dose Level | | Total Path of | Average Speed | Resting | Motion |
|---|---|---|---|---|---|---|
| Group | (mg/kg) | | Motion (mm) | (mm/s) | Time (s) | Time (s) |
| Negative | 0 | Mean | 16456.89 | 27.43 | 360.38 | 239.52 |
| Control | | SD | 3134.68 | 5.22 | 30.55 | 30.64 |
| | | n | 10 | 10 | 10 | 10 |
| Low | 25 | Mean | 14632.50 | 24.39 | 377.56 | 222.43 |
| | | SD | 2272.09 | 3.79 | 29.09 | 29.07 |
| | | n | 10 | 10 | 10 | 10 |
| Middle | 75 | Mean | 14508.22 | 24.18 | 378.85 | 221.11 |
| | | SD | 2142.30 | 3.57 | 29.66 | 29.67 |
| | | n | 10 | 10 | 10 | 10 |
| High | 250 | Mean | 18238.50 | 30.40 | 348.36 | 251.64 |
| | | SD | 8966.37 | 14.95 | 76.59 | 76.59 |
| | | n | 10 | 10 | 10 | 10 |

Note:
“n” indicates the number of animals.

During the study, no test article-related changes were noted in the total path, the time, number of times that rats passed the former platform quadrant, and latency period in animals of the 25, 75, and 250 mg/kg groups. Compared to the concurrent negative control group of the same sex, a statistically significant decrease in latency period was noted in females of the 25 mg/kg group (25.95 s vs 50.09 s), but it was not considered test article-related as it was only noted in a single sex and had no dose-dependence. Results of assessment of learning and recall of a novel response are summarized in Tables 6 and 7 below.

TABLE 6
Summary of Morris Water Maze Test - Males.

| | | | | | The Number of | |
|---|---|---|---|---|---|---|
| | Dose | | | The Time that | Times that the Rat | |
| | Level | | Total Path of | the Rat Passes | Passes The | Latency |
| Group | (mg/kg) | | Motion (mm) | The Platform (s) | Platform (time) | Period (s) |
| Negative | 0 | Mean | 36.28 | 0.20 | 0.5 | 48.86 |
| Control | | SD | 47.80 | 0.27 | 0.7 | 17.65 |
| | | n | 10 | 10 | 10 | 10 |
| Low | 25 | Mean | 9.54 | 0.05 | 0.2 | 54.74 |
| | | SD | 22.76 | 0.11 | 0.4 | 14.91 |
| | | n | 10 | 10 | 10 | 10 |
| Middle | 75 | Mean | 45.32 | 0.17 | 0.6 | 48.01 |
| | | SD | 73.20 | 0.28 | 1.0 | 19.59 |
| | | n | 10 | 10 | 10 | 10 |
| High | 250 | Mean | 57.77 | 0.27 | 0.8 | 47.73 |
| | | SD | 71.90 | 0.37 | 0.9 | 13.58 |
| | | n | 10 | 10 | 10 | 10 |

Note:
“n” indicates the number of animals.

TABLE 7
Summary of Morris Water Maze Test - Males.

| | | | | | The Number of | |
|---|---|---|---|---|---|---|
| | Dose | | | The Time that | Times that the Rat | |
| | Level | | Total Path of | the Rat Passes | Passes The | Latency |
| Group | (mg/kg) | | Motion (mm) | The Platform (s) | Platform (time) | Period (s) |
| Negative | 0 | Mean | 30.62 | 0.18 | 0.4 | 50.09 |
| Control | | SD | 57.10 | 0.35 | 0.7 | 19.17 |
| | | n | 10 | 10 | 10 | 10 |
| Low | 25 | Mean | 81.01 | 0.34 | 1.0 | 25.95* |
| | | SD | 59.91 | 0.28 | 0.7 | 20.12 |
| | | n | 10 | 10 | 10 | 10 |
| Middle | 75 | Mean | 46.40 | 0.25 | 0.7 | 43.96 |
| | | SD | 73.88 | 0.47 | 1.1 | 22.75 |
| | | n | 10 | 10 | 10 | 10 |
| High | 250 | Mean | 48.62 | 0.22 | 0.6 | 47.65 |
| | | SD | 74.58 | 0.32 | 0.8 | 16.83 |
| | | n | 10 | 10 | 10 | 10 |

Note:
“n” indicates the number of animals; Vs Negative Control,
*P ≤ 0.05.

During the study, no test article-related changes were noted in hematology in animals of the 25, 75, and 250 mg/kg groups. Compared to the concurrent negative control group of the same sex, statistically significant changes in hematology were noted in males of the 25, 75, and 250 mg/kg groups, including decreased Baso (10⁹/L) on Day 51 and decreased RBC on Day 78 in males of the 25 mg/kg group, decreased Retic (10¹²/L) on Day 51 and decreased Neut (%) on Day 78 in males of the 75 mg/kg, and decreased Baso (10⁹/L) on Day 51 in males of the 250 mg/kg group. These changes were not considered test article-related as they were slight and had no dose-dependence or regularity.

During the study, no test article-related or toxicologically significant changes were observed in clinical chemistry parameters in animals of the 25, 75, and 250 mg/kg groups. Compared to the concurrent negative control group of the same sex, no statistically significant or test article-related changes were noted in coagulation in animals of the 25, 75, and 250 mg/kg groups during the study.

During the study, no test article-related or toxicologically significant changes were observed in clinical chemistry parameters in animals of the 25, 75, and 250 mg/kg groups. Compared to the concurrent negative control group of the same sex, statistically significant changes in clinical chemistry were noted in animals of the 25, 75, and 250 mg/kg groups, including decreased ALT on D78 and decreased Glu and Na+ on Day 51 in males of the 25 mg/kg group, increased TP and decreased Glu on Day 51 in males of the 75 mg/kg group, increased AST and LDH, and decreased Glu and Na+ on Day 51 in males of the 250 mg/kg group, and increased ALT and UREA on Day 78 in females of the 250 mg/kg group. These changes were not considered test article-related as they were slight and independent of dose.

During the study, no test article-related changes were observed in lymphocyte subsets (CD3+, CD3+CD4+, CD3+CD8+ and CD3+CD4+/CD3+CD8+) in animals of the 25, 75, and 250 mg/kg groups. Compared to the concurrent negative control group of the same sex, statistically significant changes in lymphocyte subsets were noted in the test article groups, including decreased CD3+CD8+(decreased by 9.3%), and increased CD3+CD4+/CD3+CD8+(increased by 16.3%) in females of the 25 mg/kg group on Day 78, and increased CD3+(increased by 9.7%) in males of the 250 mg/kg group on Day 51. These changes were not considered test article-related since they were slight or dose-independent, or not consistent between males and females.

During the study, no test article-related changes were observed in the cytokines (IL-2, IL-6, TNF-α and IFN-γ) in animals of the 25, 75, and 250 mg/kg groups. Compared to the concurrent negative control group of the same sex, a statistically significant increase in IL-6 (Day 51) was noted in females of the 25 mg/kg group and a statistically significant decrease in TNF-α (Day 78) was noted in females of the 250 mg/kg group. Considering the changes were slight and the values were still within the historical background range of this species in the testing facility, the changes were not considered related to the test article.

ADAs in serum samples were analyzed using a validated MSD method. The results are summarized in Table 8 below.

TABLE 8
Statistics of Anti-Drug Antibodies.

| | Group | | | |
|---|---|---|---|---|
| | Negative | Low | Middle | High |
| | Control | Dose | Dose | Dose |
| Dose Level (mg/kg) | 0 | 25 | 75 | 250 |
| Positive Rate of Animals | 2.50 | 0.00 | 2.50 | 0.00 |
| Total Positive Rate of | | 0.83 | | |
| Animals in Dose Groups | | | | |
| Earliest ADA Onset | D 50 | N/A | D 50 | N/A |
| Titer Range | <1 | N/A | 1:4 | N/A |

In the negative control group, only an animal (Animal ID: 2275454) was ADA-positive on D50, the other animals were all ADA-negative. The SNR value of the animal sample in screening assay was 1.32, which was slightly greater than the screening cut point (1.285), the titer was low (titer <1), and no ADAs were detected in animals later, it is considered to be related to higher screening sensitivity of analysis method (50 ng/mL) and false positives of the threshold calculation, which may be false positive.

Following intravenous injection with 25, 75 and 250 mg/kg of test article to juvenile C57BL/6J mice once every week, only an animal in the middle dose group was ADA-positive with titer of 1:4, while no ADAs were detected in the animal later. No ADAs were detected in the other animals in each group. The positive rates of animals in each group were 0.00%, 2.50% and 0.00%, respectively, and there was no significant dose-relationship among the ADA incidence in each group. Immunogenicity testing results are highly dependent on the type of assay, sensitivity and specificity of the assay, drug administration route, time of sampling, animal status and many other factors. Due to the interspecies differences in the immune system, the immunogenicity testing results can only help explaining the relevant toxicology and toxicokinetic results, and cannot be extrapolated to humans.

Toxicokinetics: following the 7-week dosing period, trend of serum drug concentration over time was basically the same between genders in each test article group, and serum drug concentrations were positively correlated with dose levels. TK parameters were basically the same between genders in each test article group, and the maximum drug concentration (Cₘₐₓ) and exposure (AUCₗₐₛₜ) were both positively correlated with dose levels. After dosing for 7 consecutive weeks, there was no significant drug accumulation in each test article group. TK parameters after repeated intravenous injections of ALMB-0168 to juvenile C57BL/6J mice is shown in Table 9 below.

TABLE 9
Toxicokinetic parameters after repeated intravenous
injections of ALMB-0168 to juvenile C57BL/6J mice.

| | Dose Level | | Cₘₐₓ | SE_Cₘₐₓ | AUCₗₐₛₜ | SE_AUCₘₐₓ | |
|---|---|---|---|---|---|---|---|
| Period | (mg/kg) | Sex | (μg/mL) | (μg/mL) | (h*μg/mL) | (μg/mL) | AF |
| D 1 | 25 | Male | 357 | 11.7 | 11300 | 433 | |
| | | Female | 396 | 9.45 | 11400 | 293 | |
| | | Male/Female | 0.90 | | 1.00 | | |
| | 75 | Male | 844 | 106 | 19900 | 998 | |
| | | Female | 1070 | 122 | 19300 | 789 | |
| | | Male/Female | 0.79 | | 1.03 | | |
| | 250 | Male | 3430 | 164 | 47900 | 2170 | |
| | | Female | 3600 | 61.7 | 56800 | 2230 | |
| | | Male/Female | 0.95 | | 0.84 | | |
| D 50 | 25 | Male | 488 | 46.2 | 12400 | 219 | 1.09 |
| | | Female | 597 | 3.82 | 10900 | 294 | 0.961 |
| | | Male/Female | 0.82 | | 1.13 | | |
| | 75 | Male | 1860 | 91.4 | 24100 | 1500 | 1.21 |
| | | Female | 1830 | 44.4 | 22300 | 562 | 1.15 |
| | | Male/Female | 1.02 | | 1.08 | | |
| | 250 | Male | 5880 | 100 | 83500 | 1700 | 1.74 |
| | | Female | 5390 | 233 | 67700 | 2740 | 1.19 |
| | | Male/Female | 1.09 | | 1.23 | | |

No test article-related changes in organ weight, macroscopic and microscopic observations (including injection site) were noted in animals of the 25, 75, and 250 mg/kg groups at the end of the dosing period (Day 51) and recovery period (Day 78).

Compared to the negative control group, no statistically significant or test article-related changes were noted in sperm counts (the left cauda epididymis), motility, progressive, VAP, VSL, VCL, ALH, BCF, STR, LIN, ELO, in male animals of the 25, 75 and 250 mg/kg groups.

Abnormalities in sperm morphology in male rats of Groups 1 to 4 included no-hook, acephalia, tailless, fold tail, prehensile tail, double heads and amorphous head. No test article-related changes were observed in sperm malformation rate and various sperm malformations in male animals of the 25, 75 and 250 mg/kg groups. Compared to the negative control group, a statistically significant increase in the sperm amorphous head of male rats in the 25 and 75 mg/kg groups on Day 51. It was not considered test article-related due to lack of dose-relationship, and there was no difference in the total malformation rate.

Compared to the negative control group, no statistically significant or test article-related changes in estrous cycle irregularities were noted in females of the 25, 75 and 250 mg/kg groups.

No test article-related changes were noted in BMD of femur in animals of all test article groups after the dosing and recovery period. No statistically significant changes in BMC were noted in females at the end of dosing and recovery periods, and in males at the end of the dosing period, but statistically significant decreases in BMC were noted in males of the 25 and 75 mg/kg groups at the end of the recovery period. Based on individual data, the BMC values of animals in each test article group were similar to that of the control group, and there was no statistically significant change in BMD. In combination with animal body weight data, the body weights of male animals in the 25 and 75 mg/kg groups before administration (Day −1) and at the end of recovery period at euthanasia (Day 78) were lower compared with the current negative control group, and decreases in BMC were considered possibly related to which was considered to be a decrease in bone volume resulted by low body weight, resulting in a decrease in BMC. Considering that there was no dose-dependence, the changes in BMD were not considered related to the test article, and were not-related or to be toxic reactions.

During the study, no test article-related changes were observed in organ weights, organ-to-body/brain weight ratios in animals of the 25, 75, and 250 mg/kg groups. Compared to the concurrent negative control group of the same sex, statistically significant changes in organ weight were noted in the test article groups, but they were not considered test article-related since there were no consistent changes between absolute and relative weight (organ-to-brain/body weight ratio), or the changes had no dose-dependence, and there were no corresponding microscopic lesions. Changes in organ weight were as follows:
25 mg/kg: a decrease in liver (including gallbladder)-to-brain weight ratio in males on Day 51;
75 mg/kg: an increase in brain-to-body weight ratio and a decrease in liver (including gallbladder)-to-brain weight ratio in males on Day 51, and decreases in absolute weight, organ-to-body, and organ-to-brain weight ratios of adrenal glands in males on Day 78;
250 mg/kg: an increase in brain-to-body weight ratio of and decrease in the organ-to-brain weight ratio of liver with gallbladder on Day 51 in males.

There were no test article-related gross lesions in animals at the terminal necropsy (Day 51) and the recovery necropsy (Day 78). Black discoloration in spleen and cyst in the right kidney were observed in some mice at the terminal and recovery necropsies, and these gross lesions correlated with minimal hemosiderin pigmentation in the red pulp of spleen, and renal pelvis dilation of kidney. The lesions were noted in both the test article groups and the negative control group, or were only noted in one animal without dose-dependence, or were common in juvenile C57BL/6J mice. Therefore, they were not considered related to the test article.

There were no test article-related microscopic findings in animals at the terminal necropsy (Day 51) and the recovery necropsy (Day 78). Microscopic findings noted at the main and recovery necropsies were not severe, or were common in juvenile C57BL/6J mice, or were also present in the negative control group without a dose-dependence, and were therefore considered not related to the test article.

No abnormal findings were noted at the administration site in animals of Groups 1 to 4 during the study. No irritant reactions were noted at the administration site in macroscopic observation in animals of the 25, 75 and 250 mg/kg groups, and no irritant reactions were noted at the administration site in microscopic examinations in animals of the 250 mg/kg group.

In sum, during the study, no test article-related changes were observed in clinical observations, including body weight, body weight gain, growth velocity per unit time, food consumption, body temperature, ophthalmologic examinations, hematology, coagulation, clinical chemistry, lymphocyte subset, cytokines, growth and physical development, behavioral development, BMD and BMC, male sperm parameters, and female estrous cycle examination in animals of the 25, 75 and 250 mg/kg groups. Immunogenicity: anti-drug antibodies were detected in one animal of the 75 mg/kg group on Day 50 with a titer of 1:4, and no ADAs were detected in this animal later. No ADAs were detected in other animals. The presence of anti-drug antibodies was considered to be caused by non-specific reaction since anti-drug antibodies were only detected in one animal with a low titer.

### CONCLUSION

Following intravenous injections of ALMB-0168 at 25, 75 or 250 mg/kg once a week for 7 consecutive weeks (8 doses in total) to juvenile C57BL/6J mice, no toxic effects and target organs were noted. Therefore, the no observed adverse effect level (NOAEL) was considered 250 mg/kg. The AUCₗₐₛₜ and Cₘₐₓ in males at 250 mg/kg on Day 50 were 83500 h*μg/mL and 5880 μg/mL, respectively, and the AUCₗₐₛₜ and Cₘₐₓ in females at 250 mg/kg on Day 50 were 67700 h*μg/mL and 5390 μg/mL, respectively.

After ALMB-0168 was intravenously injected to juvenile C57BL/6J mice at the concentrations of 1.25, 3.75 or 12.5 mg/mL, no test article-related irritations were noted at administration site.

### Example 2. A Multicenter, Single-arm, Open-label Phase I/II Clinical Study to Evaluate the Safety and Efficacy of ALMB-0168 in Patients with Osteosarcoma

### Objectives and Endpoints of The Study

### Part I of The Study

The primary objective of Part I is to evaluate the safety and tolerability of ALMB-0168 in patients with osteosarcoma who are at least 12 years old and have failed the standard of care and to explore the recommended Phase II dose (RP2D).

The secondary objectives of Part I are:
to evaluate the pharmacokinetic (PK) profile of ALMB-0168;
to evaluate the efficacy of ALMB-0168 in the treatment of patients with osteosarcoma, and
to evaluate the immunogenicity of ALMB-0168.

The primary endpoints of Part I are:
physical examination, vital signs, pulse oxygen saturation, safety laboratory tests, ECG, and occurrence of AEs, SAEs and DLTs; and
MTD (if any).

The secondary endpoints of Part I are:
pharmacokinetic indicators: plasma concentrations and/or PK parameters of ALMB-0168, including but not limited to AUC₀₋ₜ, AUC_{0-inf}, Cₘₐₓ, C_{trough}, Tₘₐₓ, CL, and λz, t_{1/2};
efficacy indicators: objective response rate (ORR), progression-free survival (PFS), duration of response (DoR), time to response (TTR), disease control rate (DCR), 4-month progression-free-survival rate (4m-PFSR), 6-month progression-free-survival rate (6m-PFSR), overall survival (OS); rate of change from baseline in alkaline phosphatase (ALP) or lactate dehydrogenase (LDH); skeletal related events (SREs); change from baseline in Numeric Rating Scale (NRS) and Europe Health-related Quality of Life Questionnaire 5 Dimensions (EQ-5D) scores; bone mineral density of the lumbar spine, hip, and femur and neck; dose and frequency of morphine use; and
incidence of anti-drug antibodies (ADA).

### Part II of The Study

The primary objective of Part II is to further evaluate the safety and efficacy of ALMB-0168 in patients with high-grade osteosarcoma who are at least 12 years old and failed after standard of care to further determine RP2D.

The secondary objectives of Part II are:
to further evaluate the pharmacokinetic (PK) profile of ALMB-0168;
to evaluate the efficacy of ALMB-0168;
to further evaluate the immunogenicity of ALMB-0168; and
to evaluate the correlation between the biomarkers (CD39 and CD73) and clinical efficacy.

The primary endpoints of Part II are:
physical examination, vital signs, pulse oxygen saturation, safety laboratory tests, ECG, and occurrence of AEs and SAEs; and
efficacy indicators: 4-month progression-free-survival rate (4m-PFSR).

The secondary endpoints of Part II are:
efficacy indicators: objective response rate (ORR), progression-free-survival (PFS), 6-month progression-free-survival rate (6m-PFSR), duration of response (DoR), time to response (TTR), disease control rate (DCR), overall survival (OS); rate of change from baseline in alkaline phosphatase (ALP) or lactate dehydrogenase (LDH); skeletal related events (SREs); change from baseline in Numeric Rating Scale (NRS) and Europe Health-related Quality of Life Questionnaire 5 Dimensions (EQ-5D) scores; bone mineral density (BMD) of the lumbar spine, hip, and femur and neck; dose and frequency of morphine use;
pharmacokinetic indicators: plasma concentration and/or PK parameters of ALMB-0168, including but not limited to Cₘₐₓ and C_{trough};
incidence of anti-drug antibodies (ADA); and
correlation between the expression of biomarkers CD39 and CD73 proteins in tumor tissue and the efficacy.

### Study Design

### Overall Design

This is a multicenter, open-label, single-arm Phase I/II clinical study to evaluate the safety and preliminary efficacy of ALMB-0168 in patients with osteosarcoma who have failed the standard of care.

The Phase I of this study will be conducted in two stages (PART I: dose escalation stage and PART II: dose expansion stage), and all subjects will receive multiple cycles of intravenous infusion of ALMB-0168, once every 3 weeks. An accelerated titration and a standard “3+3” dose escalation design will be used in the dose escalation stage. The escalation method of accelerated titration will be used for the first 2 dose groups (1 mg/kg, 3 mg/kg). One subject will be enrolled in each group, and the dose will be escalated to the next group if the subject does not experience a Grade ≥2 TEAE related to the investigational drug within 3 weeks after the first dose. If the subject experienced a Grade ≥2 TEAE related to the investigational drug, the 3+3 dose escalation method will be used for the dose level. The dose escalation method of 3+3 will be used starting from the 3rd dose group (6 mg/kg). The dose escalation of each subsequent cohort will be judged according to the incidence of ALMB-0168-related adverse events (AEs) evaluated by NCI-CTCAE v5.0 within 3 weeks after the first dose (DLT observation period). Dose escalation will continue until ≥⅓ of the subjects at the dose level (when ≤6 subjects have been assessed) experience a DLT. The tolerable level before the dose level will be considered the MTD, and the safety can be re-assessed by selecting an appropriate dose between the two doses after discussion and communication between the Sponsor and the Investigator.

The selection of RP2D from the tolerated dose range will be based on short- and long-term safety information, as well as findings from preclinical dose-finding studies, PK, PD and anti-tumor activity studies. For dose groups that have completed the DLT observation period, after a comprehensive analysis of the subjects' benefits by the Investigator and the Sponsor based on the assessment of previous clinical data, they can be expanded to a maximum of 8 subjects (excluding the MTD expansion cohort, and up to 10 subjects can be enrolled for MTD) to further provide information on safety, PK profile and anti-tumor activity, and the safety data of the expanded subjects will not be used as the basis for determining the MTD.

In the dose expansion stage, an expansion study will be conducted in 1-3 dose groups to explore the initial efficacy in patients with high-grade osteosarcoma who have failed the standard of care, and further describe the safety, PK properties and so on of ALMB-0168. Based on the comprehensive analysis of the existing clinical study data, since good efficacy and safety were observed in the 6 mg/kg dose group, after discussion between the PI and the Sponsor, it is decided to first perform expansion in the 6 mg/kg dose group. After a certain number of subjects with evaluable efficacy are accumulated in the 6 mg/kg dose group, the Investigator and the sponsor will jointly discuss and decide whether to continue expansion in the 6 mg/kg dose group or in one or more other dose groups that have completed DLT observation to determine the RP2D. A maximum of 60 subjects are planned to be enrolled in each dose group, and whether to increase subjects will be decided after efficacy and safety data are obtained.

All subjects will sign the ICF prior to any specific study activity. Eligible subjects will be treated at the dose level determined in the dose escalation stage after enrollment. Theoretically, 21 days (3 weeks) is defined as 1 treatment cycle in this study. All subjects will receive an intravenous infusion of ALMB-0168 every 3 weeks. The subjects will receive multiple cycles of treatment until progressive disease or anti-tumor treatment becomes necessary. All subjects will be closely monitored for safety.

All subjects will be followed in person within 28 days after the last treatment, followed by a one-year follow-up to assess long-term outcomes.

### Dose Escalation Design

### Dose-Limiting Toxicity

Dose Limiting Toxicity (DLTs) are defined as the following toxic responses related to the investigational drug ALMB-0168 that occur during the first treatment cycle (21 days):
Grade 4 neutropenia lasting for ≥7 days; or Grade ≥3 neutropenia with fever (ANC <1000/mm3 with single temperature ≥38.3° C. or persistent temperature ≥38° C. for more than 1 h) or with infection;
Grade 4 thrombocytopenia or Grade 3 thrombocytopenia with hemorrhage;
Grade ≥3 hepatotoxicity: ALT or AST ≥5× upper limit of normal (ULN) (for subjects with normal baseline) or ≥5× baseline value (for subjects with combined liver metastasis and abnormal baseline); or bilirubin ≥3×ULN (for subjects with normal baseline value) or ≥3× baseline value (for subjects with abnormal baseline value);
Grade ≥3 nephrotoxicity: Serum creatinine ≥3×ULN (for subjects with normal baseline value) or ≥3× baseline value (for subjects with abnormal baseline value);
Grade ≥3 cardiotoxicity: QTc ≥500 ms or QTc increase ≥60 ms;
Other Grade ≥3 non-hematological toxicities, but excluding:
   Grade 3 nausea and/or vomiting, lasting less than 72 h in the absence of prophylactic and effective antiemetic treatment;
   Fatigue or asthenia lasting less than 7 days;
   Transient and asymptomatic laboratory test abnormalities lasting less than 3 days;
   Alopecia, rash and diarrhea that are controllable under standard supportive treatment.
Toxic responses for which the Investigator judges that ALMB-0168 should be permanently discontinued.

The above AEs will be graded according to the National Cancer Institute-Common Terminology Criteria for Adverse Events (NCI-CTCAE) Version 5.0.

### Maximum Tolerated Dose

The dose escalation stage in this study will be performed using an accelerated titration and a standard “3+3” dose escalation design: The escalation method of accelerated titration will be used for the first 2 dose groups (1 mg/kg, 3 mg/kg). One (1) subject will be enrolled in each group. If the subject does not experience DLT or intolerance during the treatment period, the first dose to subjects at the next dose level can be started. If a subject in the accelerated titration group experiences moderate toxicity (including the following drug-related AEs requiring treatment: 1 grade 3 AE not belonging to DLT, or 2 grade ≥2 AEs), the principle of “3+3” should be followed for this dose group and subsequent dose groups. The dose escalation method of 3+3 will be used starting from the 3rd dose group (6 mg/kg). The dose escalation of each subsequent cohort will be judged according to the incidence of ALMB-0168-related adverse events (AEs) evaluated by NCI-CTCAE v5.0 within 3 weeks after the first dose (DLT observation period). Dose escalation will continue until ≥⅓ of the subjects experience DLT (when ≤6 subjects are assessed) at the dose level. The tolerable dose level before the dose level will be considered the MTD.

After cycle 1, all Grade ≥4 adverse events during the extension period will also be one of the factors in determining dose escalation and MTD.

### Estimation of Initial Dose

The results of toxicity studies in non-human primates and mice will be used to calculate the safe clinical starting dose for this study. The specific method is consistent with the guidance provided in ICH S9. The no-observed-adverse-effect level (NOAEL) of ALMB-0168 is 250 mg/kg in both non-human primates and rodents. Considering the novel target of ALMB-0168, a safety factor exceeding 100×NOAEL will be used to ensure that the first dose will not cause adverse effects in humans. The final starting dose of the drug in this first-in-human study is 1 mg/kg.

### Determination of Maximum Escalation Dose

In the non-GLP dose range-finding study in non-human primates (NIP), ALMB-0168 was infused intravenously to cynomolgus monkeys at doses of 20, 60, and 200 mg/kg weekly, and no adverse reactions were observed in any dose group in the following 2 weeks. Subsequently, in a GLP repeat-dose toxicology study, cynomolgus monkeys were infused intravenously at doses of 25, 75, and 250 mg/kg once weekly for 6 weeks, followed by a 4-week recovery period. No significant adverse reaction were found. In addition, in the GLP repeated-dose toxicology study in mice, mice were repeatedly dosed at 25, 75 and 250 mg/kg weekly for 6 weeks, and no significant adverse reactions were observed after 4 weeks of observation and recovery. Therefore, the NOAEL of ALMB-0168 was 250 mg/kg in both NHP and rodents.

In the preclinical efficacy study, infusion of ALMB-0168 at 25 mg/kg once a week significantly inhibited human breast cancer growth in a mouse xenograft model. In a mouse model of in situ osteosarcoma, once-weekly administration of 15-25 mg/kg ALMB-0168 significantly inhibited tumor growth in the tibia and prolonged life span. The maximum escalation dose in the clinical study is tentatively set at 36 mg/kg, which will also be adjusted, if necessary, based on the PK, PD and safety data obtained for each dose group of the subjects in Australia and China.

### Administration Route and Frequency

21 days (3 weeks) is defined as 1 treatment cycle in this study. All subjects will receive an intravenous infusion of ALMB-0168 every 3 weeks. Subjects will receive multiple cycles of treatment until progressive disease or intolerable toxicity. All subjects will be closely monitored for safety.

All subjects will be followed in person within 28 days after the last treatment, followed by a one-year follow-up to assess long-term outcomes.

### Setting of Dose Escalation Group

Dose escalation levels will be based on investigational drug-related adverse events observed during the first cycle of dosing and dose escalation gradients as specified in the dose escalation setup table. If 2 or more subjects experience an investigational drug-related Grade ≥2 TEAE in the first dosing cycle, or 1 subject experiences an investigational drug-related Grade ≥3 TEAE, the magnitude of dose escalation between cohorts may be reduced as necessary, e.g., from 100% dose escalation to no more than 50% dose increase from the previous dose level, as mutually discussed between the Investigator and the Sponsor. ALMB-0168 will be escalated in no more than 33% increase at dose levels of 24 mg/kg and above. In case of any problem in the safety assessment, the dose escalation magnitude can be appropriately reduced by the same procedure. It is expected that no more than 8 dose groups will be evaluated during the study. The number and dose level of the actual dose group will depend on the maximum tolerated dose (MTD) and the safety profile observed during the study. If necessary, the Investigator and the Sponsor may discuss the intermediate dose escalation levels. If the safety and tolerability of this dose group are still good when the dose is escalated to the pre-specified maximum dose group, the Investigator and the Sponsor will jointly decide whether to explore the higher dose. See Table 10 below for the dose group setting in the specific dose-escalation stage.

TABLE 10
Specific Dose Escalation Setup of Part I.

| | Dose | Percentage Increase from | Number of |
|---|---|---|---|
| Dose Level | (mg/kg) | Previous Dose Level | Subjects (n) |
| 1 | 1 | — | 1^{a} |
| 2 | 3 | 200 | 1^{a} |
| 3 | 6 | 100 | 3-6 |
| 4 | 12 | 100 | 3-6 |
| 5 | 18 | 50 | 3-6 |
| 6 | 24 | 33 | 3-6 |
| 7 | 30 | 25 | 3-6 |
| 8 | 36 | 20 | 3-6 |
| Dose expansion | XXX | | *10 |
| at MTD | (MTD) | | |

1) ^{a}The escalation method of accelerated titration will be used for the first 2 dose groups (1 mg/kg, 3 mg/kg). One subject will be enrolled in each group, and the dose will be escalated to the next dose group if the subject has no Grade ≥2 TEAE related to the investigational drug. If the subject experienced a Grade ≥2 TEAE related to the investigational drug, the 3 + 3 dose escalation method will be used for the dose level.
2) The MTD cohort will include a maximum of 10 subjects, including 3-6 subjects in the dose escalation stage, to determine safety.
3) The dose level and percentage increase from the previous dose level for each dose group will be adjusted based on the study data from the first-in-human clinical study in Australia (ALMB-0168-AU-101).
4) Dose groups that have completed the DLT observation period can be expanded to a maximum of 8 subjects (excluding the MTD expansion cohort, and up to 10 subjects can be enrolled for MTD) after the Investigator and the Sponsor have evaluated the previous clinical data and analyzed the benefits of the subjects.

### Dose Escalation Principle

Accelerated titration and standard “3+3” escalation design will be used in the dose escalation stage: The escalation method of accelerated titration will be used for the first 2 dose groups (1 mg/kg, 3 mg/kg), and the dose escalation method of 3+3 design will be used starting from the 3rd dose group (6 mg/kg). At least 3 subjects will be enrolled in each dose group, and a judgment will be made within 3 weeks after the 3 subjects have completed the first dose at the dose level (DLT observation period) based on adverse events (AEs) related to ALMB-0168 evaluated according to NCI CTCAE Version 5.0. If none of the three subjects experience a DLT, the next group will be treated at the next dose level according to the dose escalation rule. At each dose level, if one subject experiences a DLT, another 3 subjects will be enrolled. Dose escalation will continue until ≥⅓ of the subjects experience DLT (when ≤6 subjects are assessed) at the dose level. The tolerable dose level before the dose level will be considered the MTD. The selection of RP2D from the tolerated dose range will be based on short- and long-term safety information, as well as findings from preclinical dose-finding studies, PK and anti-tumor activity studies. For dose groups that have completed the DLT observation period, after a comprehensive analysis of the subjects' benefits by the Investigator and the Sponsor based on the assessment of previous clinical data, they can be expanded to a maximum of 8 subjects (excluding the MTD expansion cohort, and up to 10 subjects can be enrolled for MTD) to further provide information on safety, PK profile and anti-tumor activity, and the safety data of the expanded subjects will not be used as the basis for determining the MTD.

### Dose Expansion Stage

In the expansion stage, an expansion study will be conducted in 1-3 dose groups, with a maximum of 60 patients with high-grade osteosarcoma enrolled in each dose group to preliminarily explore the efficacy, further describe the safety and PK properties of ALMB-0168, explore the efficacy, clarify PR2D, etc. Based on the comprehensive analysis of the existing clinical study data, since good efficacy and safety were observed in the 6 mg/kg dose group, after discussion between the PI and the Sponsor, it is decided to first perform expansion in the 6 mg/kg dose group. After a certain number of subjects with evaluable efficacy are accumulated in the 6 mg/kg dose group, the Investigator and the sponsor will jointly discuss and decide whether to continue expansion in the 6 mg/kg dose group or in one or more other dose groups that have completed DLT observation to determine the RP2D. At this stage, a maximum of 60 subjects are planned to be enrolled in each dose group, and whether to increase subjects will be decided after efficacy and safety data are obtained.

### Definition of End of Study

The end-of-study is defined as the completion of the study by the last subject if he/she has completed all stages of the study, including the last scheduled procedure listed for the final visit. The maximum follow-up time for this study is 12 months. Therefore, the end-of-study is defined as the death of the last subject in the study or completion of all procedures listed in the study flow chart (i.e., 12 months follow-up), whichever occurs first.

### Study Population

### Inclusion Criteria

1. Patients with high-grade osteosarcoma confirmed by histopathology.

2. Defined according to different stages:
a) Phase I PART I: Osteosarcoma patients with bone lesions (primary or metastatic) who failed the standard of care, including but not limited to:
   i) Stage III osteosarcoma (as defined by the Enneking staging system) with bone metastasis, with or without lung metastasis;
   ii) Unresectable locally advanced osteosarcoma;
   iii) Multifocal osteosarcoma.
b) Phase I PART II: patients with high-grade osteosarcoma who failed the standard of care.

The standard of care failure is defined as progressive disease during or within 6 months after therapy with first-line or above drugs (including high-dose methotrexate, doxorubicin [cumulative dose ≥350 mg/m²], and cisplatin); for those with progressive disease for more than 6 months, the Investigator should conduct a risk-benefit assessment and obtain the consent of the subject or his/her guardian.

3. Patients aged ≥12 years, regardless of gender.

4. Patients with an ECOG (Eastern Cooperative Oncology Group) PS score of 0, 1, or 2.

5. Patients with measurable or non-measurable lesions as per RECIST v1.1. Non-measurable lesions should be confirmed by conventional imaging techniques, including isotope bone scans, CT or MRI. Subjects included in Phase I PART II should have at least one measurable lesion confirmed by CT or MRI at baseline.

6. Patients with main system functions as follows:
a) Bone marrow reserve: absolute neutrophil count (ANC) ≥1.5×109/L; platelet count ≥75×109/L; hemoglobin ≥9 g/dL, no transfusion in the last 14 days (subjects requiring transfusion);
b) Hepatic function: total bilirubin ≤1.5× upper limit of normal (ULN) (unless the patient experiences Grade 1 bilirubin elevation due to Gilbert's disease or a similar syndrome with slow bilirubin binding). Transaminases (AST/SGOT and/or ALT/SGPT)≤3×ULN (liver metastasis ≤5×ULN);
c) Renal function: normal serum creatinine ≤1.5 mg/dL (133 mol/L) or calculated creatinine clearance ≥50 mL/min (Cockroft-Gault formula);
d) Coagulation: defined as an International Normalized Rate (INR)≤2.

7. Female subjects of childbearing potential must have a negative serum pregnancy test within 7 days prior to the first dose and must be non-lactating. Male subjects with a female partner of childbearing potential and female subjects of childbearing potential must use two acceptable methods of contraception, including 1 barrier method, during study participation and within 3 months after the last dose. Male subjects must avoid donating sperm during study participation; female subjects must avoid donating eggs during the study.

8. Expected survival ≥3 months.

9. Subjects (or their guardians) are able to understand the whole process of this study and voluntarily participate in the study and sign the ICF. For subjects who are unable to understand the ICF or accurately express their will, their guardians may sign the ICF with the subject's fingerprint.

### Exclusion Criteria

1. Patients with clinically confirmed brain metastasis, metastases to meninges, spinal cord compression, or brain or central nervous system injury.

2. Patients currently with the following cardiac conditions or previously in the past 6 months:
a. left ventricular ejection fraction (LVEF)<45% as measured by echocardiography (ECHO);
b. screening electrocardiogram (ECG) shows QTc interval >470 ms for females and >450 ms for males;
c. unstable angina;
d. congestive heart failure (New York Heart Association [NYHA]>Grade 2);
e. acute myocardial infarction;
f. poorly controlled arrhythmia;
g. acute coronary syndrome; and
h. stent placement.

3. Patients with uncontrolled hypertension. (Systolic blood pressure [SBP]>160 mmHg or diastolic blood pressure [DBP]>100 mmHg (blood pressure above these levels must be controlled to below these levels with drugs before the study initiation).

4. Patients with serious active infections (systemic intravenous antibiotics used within 14 days, but oral antibiotics permitted) not well controlled, or other serious illness that prevents the subject from receiving the study drug.

5. Subjects who are positive for anti-HIV antibody or anti-Treponema pallidum specific antibody tests, or with active hepatitis B (defined as positive for HBsAg and HBV DNA ≥500 IU/mL) or with active hepatitis C (defined as positive for HCV antibody and HCV-RNA).

6. Patients with other active tumors, or a history of treatment for invasive tumors within 3 years. Subjects with a definite local treatment for a stage I tumor who are considered unlikely to have relapse are eligible for the study. Patients with a prior history of treatment for carcinoma in situ (e.g., non-invasive) and a history of non-melanoma skin cancer are eligible for the study.

7. Anti-tumor treatment (including but not limited to chemotherapy, immunotherapy and targeted therapy, etc., as well as unmarketed anti-tumor treatment) before the first dose ≤28 days or within 5 half-lives of the drug, whichever is shorter, or with associated side effects ≥Grade 1 (as per NCI-CTCAE v5.0), except for alopecia.

8. Subjects who have participated in clinical studies of other drugs within 28 days prior to the first dose or within 5 half-lives of ALMB-0168, whichever is shorter, and are enrolled for treatment.

9. Patients who have received extensive radiotherapy (including radioisotope therapy with strontium 89)≤28 days prior to initiation of treatment or local radiotherapy ≤7 days prior to treatment, or have not recovered from the side effects of such treatment.

10. Patients who have undergone major surgery 28 days prior to initiation of treatment or outpatient surgery 7 days prior to initiation of treatment, except for endovascular stent placement.

11. Patients who have received Chinese herbal medicine or Chinese patent medicine with anti-tumor activity within 7 days before administration.

12. Patients with Grade ≥3 allergic reactions to human monoclonal antibodies or fusion proteins in the past and/or with relevant contraindications.

13. Pregnant women. Lactating women should stop breastfeeding before signing the ICF.

14. Other conditions in which the patient was considered inappropriate in this study by the Investigator.

### Lifestyle Consideration

Alcohol consumption should be prohibited from 24 hours prior to each treatment cycle until the collection of the final pharmacokinetic (PK) samples. Smoking subjects should be advised not to use any nicotine-containing products (including nicotine patches) at the study site. Strenuous exercise should be prohibited within 1 hour prior to each blood collection for laboratory tests. Subjects may participate in some relaxed entertainment activities (e.g., watching TV, reading) during the study.

### Screen Failures

Screening failure is defined as subjects who agreed to participate in the clinical study but are not randomly assigned to receive the investigational intervention or to be included in the study. Only a very small fraction of the screening failure information is required to ensure transparent reporting of the subjects failing the screening, so as to meet the requirements for publication of the Consolidated Standards of Reporting Trials (CONSORT). It can also be used for supervision of the supervision authority. This part of information includes demographics, screening failure details, inclusion criteria, and any serious adverse event (SAE).

### Intervention

### ALAM-0168

ALMB-0168 is a humanized monoclonal antibody prepared from a recombinant CHO cell strain under GMP conditions. Similar to typical humanized antibodies, ALMB-0168 consists of 2 identical heavy chains (IgG4 variants) and 2 identical light chains (κchains) covalently linked by 4 pairs of disulfide bonds. The heavy and light chains consist of 446 and 219 amino acid residues, respectively. Based on the total amino acid composition, the molecular weight is 146.2 kDa. Similar to most antibodies, the heavy chains are glycosylated at the conserved glycosylation site in the constant region.

ALMB-0168 is a novel Cx43 humanized IgG4 monoclonal antibody targeting the Cx43 hemichannels of osteocytes. Initial antibodies are obtained by immunizing mice with human Cx43. Mouse antibodies are then humanized using a common humanization approach, i.e., complementary determining region (CDR) transplantation (Bowers et al., J Biol Chem.; 288 (11): 7688-7696 (2013) (also known as modification) and germline sequence modeling.

ALMB-0168 is the first drug designed to activate the Cx43 hemichannels and inhibit the growth of primary and metastatic bone tumors by activating Cx43 hemichannels to release critical substances such as ATP into the extracellular environment.

The physicochemical properties of ALMB-0168 are shown in Table 11 below.

TABLE 11
Physicochemical properties of ALMB-0168.

| Category | Characteristics |
|---|---|
| Subtype of immunoglobulin | IgG4 |
| Appearance | Colorless or pale yellow clear liquid, with |
| | slight opalescence |
| Solubility | 25 mg/ml in formulation buffer (pH 5.5) |
| Isoelectric point (pI) | 8.1 |
| Theoretical extinction | 1.531 |
| coefficient | |

ALMB-0168 is a sterile, pyrogen-free injection of monoclonal Ab (mAb) against human connexin 43, provided by the Sponsor, AlaMab Therapeutics (Shanghai) Inc. Each vial contains 6 ml (150 mg) of 25 mg/ml ALMB-0168 mAb, and stored at −20±5° C., protected from light and avoided from shaking. ALMB-0168 will be administered by intravenous infusion for at least 30 minutes, and all subjects will receive an intravenous infusion of ALMB-0168 alone every 3 weeks. Subjects will receive multiple cycles of treatment until progressive disease or intolerable toxicity.

Currently, there are no drugs of the same class for ALMB-0168. Chronic active hemorrhagic inflammation and lymphopenia in the central and marginal zones of the spleen were observed in preclinical animal studies. Therefore, subjects should be closely observed for infusion reaction, infection, flu-like symptoms, rash, myalgia and arthralgia, fever and chills, and toxicities of the heart, liver, kidney and other organ systems during the study. Subjects receiving the first ALMB-0168 infusion must be observed for at least 48 hours post-infusion in an area with resuscitative equipment and emergency medications.

During the study, if subjects experience adverse reactions, the Investigator should provide necessary supportive treatment according to clinical needs, and allow the suspension of the investigational drug and/or the dose adjustment.

If all investigational drug related AEs occurring in the previous treatment cycle do not return to baseline levels or to Grade 1 on the day of subsequent scheduled treatment, treatment should be delayed and toxic responses graded weekly. The date subjects receive the next cycle of ALMB-0168 treatment will be recorded as Day 1 of the next treatment cycle. If all investigational drug related AEs return to baseline levels or to NCI-CTCAE Grade 1 within 3 weeks, dosing may be continued.

A subject who requires a treatment delay of more than 21 days due to investigational drug related toxicities occurring in the previous treatment cycle will permanently discontinue study treatment and will complete the study by having an End-of-Treatment Visit 4 weeks after the last treatment.

### Preparation Handling Storage Responsibilities of ALMB-0168

The Sponsor should be responsible for delivering ALMB-0168 to the study site, and the Investigator or investigator's designee should sign to confirm the receipt.

ALMB-0168 can only be used in this study and can only be managed by a special person authorized by the Investigator. The Investigator/Pharmacist in charge of drug administration should distribute, recycle and destroy ALMB-0168, and maintain accurate records in accordance with the study procedures. The used investigational drugs and their packages should be disposed and destroyed as medical wastes in the study site, and the unused or expired drugs should be destroyed by the Sponsor after recovery.

The instructions for rewarming, dilution, mixing and preparation of ALMB-0168 should be recorded in the corresponding package insert.

The usage of ALMB-0168 must be in strict accordance with the clinical protocol regulations, and the dose applied to each subject should be recorded in eCRF. Investigators in the study site should check and count during drug distribution, recovery and destruction, and sign the documents of drug transfer, handover and destruction for confirmation.

### Dosage Form, Appearance, Package and Label

The label should contain the following:
Protocol No.: ALMB-0168-CN-101
Drug No.:
Investigational Drug: ALMB-0168 Injection
Strength: 150 mg, 6 ml/vial
Mfg Lot No:
Dosage Form: Intravenous injection
Shelf Life:
Indication: High-grade osteosarcoma after the failure of the standard of care
Instructions for Use: See the package insert for details
Storage Conditions: −20° C.±5° C., protected from light and shaking before use. If refrigerated, the diluted solution for infusion should be allowed to return to room temperature prior to dosing.

The label should also include the statement that “the drug is provided by AlaMab Therapeutics (Shanghai) Inc. and used for clinical studies only.”

### Product Storage and Stability

ALMB-0168 drug should be stored at −20° C.±5° C., protected from light and shaking before use. The solution for infusion should be used within 28 hours after preparation, including up to 4 hours when stored at room temperature of 18° C.-26° C. and up to 24 hours when refrigerated at 2° C.-8° C. (including the storage and infusion time of the solution for infusion in the infusion bag). The diluted solution for infusion should not be frozen or shaken.

ALMB-0168 should be ensured to keep stable within the proposed shelf life under the proposed packaging and storage conditions.

### Concomitant Medication and Treatment

Given that ALMB-0168 is a therapeutic monoclonal Ab, there is a low likelihood of drug-drug interactions between ALMB-0168 and small molecule formulations. Since ALMB-0168 is expected to degrade to amino acids and circulate to other proteins, it is unlikely to have an effect on drug metabolizing enzymes or transporters. Medications to treat conditions existing prior to study entry or new conditions emerging during the study may be administered throughout the study. Blood products, painkillers, hypnotics, and other medications for symptomatic management may be administered at the discretion of the Investigator. In addition to the drugs listed in the Exclusion Criteria (Section 5.2) and Section 6.5.1, other drugs necessary for the safety and health of subjects may be administered at the discretion of the Investigator. The Investigator may give any necessary concomitant medication or treatment for adequate supportive therapy.

All concomitant medications and treatments during the study should be recorded in the eCRF in detail. For concomitant treatments for osteosarcoma or supportive therapy, the name of the treatment, indication (reason for medication), dose, unit, frequency, start date, and stop date should be collected. For all other concomitant treatments, the treatment name, indication, start date, and stop date should be collected.

Concomitant medications should include not only drugs prescribed by the physicians, but also all OTC, Chinese herbals and vitamins.

### Prohibited/Permitted Food, Drugs and Treatment

During the study, any anti-tumor treatment (including cytotoxic chemotherapy, targeted drugs, radiotherapy, endocrine therapy, and traditional Chinese medicine with anti-tumor indications) and all other clinical investigational drugs are prohibited. If a subject needs such medications, the subject must be withdrawn from the study first.

The following drugs are allowed to be used as appropriate during the study:
(1) long-term medication required for comorbidities (such as hypertension and diabetes);
(2) hormone replacement therapy or oral contraceptives not intended for anti-tumor treatment;
(3) on the premise of protecting the interests and safety of the subjects, the Investigator should give symptomatic and supportive treatment for the toxic response of drugs or to control the tumor symptoms.

### Study Assessment and Procedures

Screening should be completed within 28 days before C1D1. Subjects who meet all inclusion criteria and none of the exclusion criteria will be assigned a group number.

Demographic data, including date of birth, gender, age and nationality, will be collected from Day-28 to Day-1.

Body height is measured at screening only. Body weight will be measured during the screening period and on CXD1 (if the body weight examination at screening is completed within 72 h pre-dose, it is not necessary to perform the body weight examination on C1D1).

### Medical History and Other Past Histories

Medical history refers to clinically significant information (or signs and symptoms, if a diagnosis cannot be confirmed) on previous and concomitant diseases that occurred before signing the ICFs, including past medical history and history of present diseases. Among them, the history of tumor disease includes the time of diagnosis, pathological classification, staging diagnosis and screening stage. All medical histories related to the subject's tumor diagnosis must be traced back to the original diagnosis. In addition, histories of smoking, alcoholism, drug abuse, surgery, or clinically significant abnormalities found in relevant examinations at screening will also be recorded as past history in the original medical records and eCRF.

### Prior Concomitant Medication, Treatment and Prior Anti-tumor Treatment

Prior medication refers to any medication taken within 28 days prior to the date of investigational drug dosing.

Concomitant medication and treatment refer to the simultaneous administration of two or more drugs/treatments, and are defined in the study as any drug/treatment administered at any time from the simultaneous/contemporaneous first dose of ALMB-0168 to 28 days after the last dose.

At each visit from the initial screening to the end of study, the Investigator should inquire about any drug/treatment the subjects have taken since the last visit. Any previous medication and concomitant treatment will be recorded in the original medical records and eCRF, and all concomitant medications will be recorded with the following information: drug/non-drug name, cause for medication, dose, unit, dosing frequency, administration route, start date and end date; concomitant non-drug treatment should include the name of non-drug treatment, treatment description, cause for treatment, start date and end date of treatment. If the reasons for use of concomitant medication and treatment comply with the definition of AEs, relevant information should be recorded in the original medical records and eCRF of the subject.

The treatment includes surgery, chemotherapy, radiotherapy, hormonal therapy, or immunotherapy. The following data should be collected: start date and end date of treatment, name of treatment regimen, dose, unit, frequency, best response and reasons for termination of treatment.

### Safety and Other Assessments

The following items are tested in laboratory:
1. hematology: Red blood cell count (RBC), hemoglobin (HGB), hematocrit (HCT), white blood cell count (WBC), platelet count (PLT) and counts and percentages of neutrophils, lymphocytes, eosinophils, basophils and monocytes, etc.
2. blood chemistry: alanine aminotransferase (ALT), aspartate aminotransferase (AST), alkaline phosphatase (ALP), lactate dehydrogenase (LDH), total bilirubin (TBIL), direct bilirubin (DBIL), total protein (TP), albumin (ALB), urea or blood urea nitrogen (BUN), creatinine (Cr), creatinine clearance (Ccr), glucose (Glu), potassium (K+), sodium (Na+), chloride (Cl−), calcium (Ca2+), magnesium (Mg2+), phosphorus (P), and carbon dioxide.
3. myocardial zymogram: creatine kinase (CK) and its isoenzyme (CK-MB);
4. urinalysis: pH, specific gravity (SG), glucose (GLU), protein (PRO), red blood cell (RBC), white blood cell (WBC), ketone (KET), occult blood, bilirubin, nitrite, and urobilinogen;
5. coagulation function: prothrombin time (PT), activated partial thromboplastin time (APTT), international normalized ratio (INR), thrombin-time (TT), and plasma fibrinogen (Fbg);
6. serum or urine pregnancy test: only for women of childbearing potential;
7. virus serology test: hepatitis B test (hepatitis B surface antigen (HBsAg), hepatitis B surface antibody (HBsAb), e antigen (HBeAg), e antibody (HBeAb), and core antibody (HBcAb), further test of HBV-DNA in patients with positive hepatitis B surface antigen), hepatitis C (hepatitis C antibody, further test of HCV-RNA in patients with positive HCV antibody), anti-HIV antibody, and anti-Treponema pallidum specific antibody).

Laboratory test time and evaluation:
(1) hematology, blood biochemistry, myocardial zymogram and urinalysis: at baseline (Day −7 to Day −1), C1D1, C1D8, C1D15, C2D1, C2D8, C2D15, D1 of each subsequent cycle, and at the EOS visit. If the C1D1 physical examination is expected to be done within 72 hours after the baseline physical examination, it can be omitted;
(2) coagulation function: Performed at screening, before dosing on D1 of each cycle, and at the end-of-study visit. If the C1D1 physical examination is expected to be done within 72 hours after the baseline physical examination, it can be omitted;
(3) serum or urine pregnancy test: Performed at screening, before dosing on D1 of each cycle and at the end-of-study visit; and
(4) virus serology: the test will be performed at screening.

The Investigator will examine all laboratory findings, assess the subject's changes from baseline in laboratory findings based on the laboratory reference range, determine the clinical significance of each abnormal laboratory value, and record it as “no clinical significance (NCS)” or “clinical significance (CS)” (no marks needed for “normal state”). Abnormal laboratory tests that occur during the study, accompanied by the following symptoms, should be listed as adverse events and recorded in the original medical records and eCRF:
directly withdrawal of subjects from the study;
leading to serious adverse consequences;
leading to obvious clinical symptoms;
clinically significant considered by the investigator.

The reviewed laboratory report will be signed and dated by the investigator.

Physical examinations, including skin, mucosa, lymph nodes, head (including skull, ears, eyes, nose, mouth, and throat), neck, chest (including thorax, breasts, lungs, and heart), abdomen (including liver, gallbladder, spleen, kidney, bladder, stomach, and intestines), spine/limbs, nervous system, and others. The examination will be performed at baseline (Day −7 to Day −1), on C1D1, C1D8, C1D15, C2D1, C2D8, C2D15, on D1 of each subsequent cycle, and at the end-of-study visit. If the C1D1 physical examination is expected to be done within 72 hours after the baseline physical examination, it can be omitted. Compared with the baseline, any clinically significant abnormality should be recorded as an AE.

Vital signs include blood pressure, respiration, resting pulse and body temperature. Examination of vital signs will be performed at baseline (Day −7 to Day −1), on C1D1, C1D8, C1D15, C2D1, C2D8, C2D15, on D1 of each subsequent cycle, and at the end-of-study visit. If the C1D1 physical examination is expected to be done within 72 hours after the baseline physical examination, it can be omitted. Compared with the baseline, any clinically significant abnormal value should be reported as an AE.

Sitting systolic and diastolic blood pressure should be measured with the same arm each time, and the measurement should be performed after the subject has rested for at least 5 min in a sitting position. All recordings will be made using standard equipment and automatic or manual measurements are acceptable; however, the Investigator should continue to perform all assessments of individual subjects using the same methods used in the first measurement.

The performance status of the subjects will be evaluated according to the ECOG Performance Status Scale. The examination will be performed at baseline (Day −7 to Day −1), on C1D1, C2D1, on D1 of each subsequent cycle, and at the end-of-study visit. If the C1D1 physical examination is expected to be done within 72 hours after the baseline physical examination, it can be omitted.

The examination will be performed at baseline (Day −7 to Day −1), at 30 min (±10 min) pre-dose and 30 min post-dose on C1D1, C1D8, C1D15, at 30 min (±10 min) pre-dose and 30 min (±10 min) post-dose on D1 of each subsequent cycle, and at the end-of-study visit. Subjects will be required to stay in a supine position for 5-10 minutes of rest as far as possible before ECG examination, and remain awake and lying on the back during ECG. The Investigator may increase the frequency of examination according to the subject's condition.

Echocardiography will be performed at baseline (Day −14 to Day −1). In addition, during the study, the investigator may perform examinations according to clinical needs. The subject should rest for at least 5 min in a supine position before UCG.

Chest CT (plain/contrast-enhanced scan) will be required during screening; the Investigator can perform examination according to clinical needs during the study.

Brain MRI, lesion MRI scan, PET-CT can be examined at baseline (Day −28 to Day −1). During the study, the investigator will perform examinations according to clinical needs.

The schedule of activities is summarized in Table 12 below.

TABLE 12
Schedule of Activities.

| | | | | | | | | | | | | | Safety | Survival |
|---|---|---|---|---|---|---|---|---|---|---|---|---|---|---|
| | Screening | | | | | | | | | | | | Follow-up | Follow-up |
| | Period | | During Treatment/Study | | | | | | | | | End-of- | Period | Period |
| | Baseline | | Cycle 1 | | | | | Cycle 2 | | | ≥3 Cycles | Treatment | 28 d ± 3 d | Once every 3 |
| | Time Point* | | | | | | | | | | | Visit (EOT) | after the | months ± 7 d |
| Procedure/Assessment | D −28 | D −7 | D 1 | D 2 | D 3 | D 8 | D 15 | D 1 | D 8 | D 15 | D 1 | EOT + 7 d | last dose | after EOT |
| Informed consent ^{a} | X | | | | | | | | | | | | | |
| Demographic data | X | | | | | | | | | | | | | |
| Inclusion/exclusion | | X | | | | | | | | | | | | |
| criteria | | | | | | | | | | | | | | |
| Medical history | X | | | | | | | | | | | | | |
| Vital signs^{b} | | X | X | | | X | X | X | X | X | X | X | | |
| Physical examination^{c} | | X | X | | | X | X | X | X | X | X | X | | |
| Height^{d} | | X | | | | | | | | | | | | |
| Body weight^{e} | | X | X | | | | | X | | | X | | | |
| ECOG score | | X | X | | | | | X | | | X | X | | |
| ECHO^{f} | X | | During the study, the Investigator will perform examinations according to clinical needs. | | | | | | | | | | | |
| 12-lead ECG^{g} | | X | X | | | X | X | X | | | X | X | | |
| Assessment of AEs ^{h} | | | X | X | X | X | X | X | X | X | X | X | X | |
| Assessment of | X | | X | X | X | X | X | X | X | X | X | X | X | |
| concomitant | | | | | | | | | | | | | | |
| medication | | | | | | | | | | | | | | |
| Hematologyⁱ | | X | X | | | X | X | X | X | X | X | X | | |
| Blood chemistry and | | X | X | | | X | X | X | X | X | X | X | | |
| myocardial zymogram^{j} | | | | | | | | | | | | | | |
| Coagulation function^{k} | | X | X | | | | | X | | | X | X | | |
| Urinalysis^{l} | | X | X | | | X | X | X | X | X | X | X | | |
| Serum or urine | | X | X | | | | | X | | | X | X | | |
| pregnancy test^{m} | | | | | | | | | | | | | | |
| Virus serology testⁿ | | X | | | | | | | | | | | | |
| PK blood sampling^{o} | | | X | X | X | X | X | X | | | X | X | X | |
| Immunogenicity blood | | | X | | | | | X | | | X | X | X | |
| sampling^{p} | | | | | | | | | | | | | | |
| Biomarker sample | X | | | | | | | | | | | | | |
| collection/acquisition ^{q} | | | | | | | | | | | | | | |
| Study treatment | | | X | | | | | X | | | X | | | |
| Evaluation of skeletal | | X | X | | | | | X | | | X | X | | |
| related events (SREs) | | | | | | | | | | | | | | |
| Dose and frequency of | | X | X | | | | | X | | | X | X | | |
| morphine use | | | | | | | | | | | | | | |
| NRS and EQ-5D | | X | X | | | X | X | X | X | X | X | X | | |
| scores | | | | | | | | | | | | | | |
| Bone mineral density | X | | BMD measured by dual-energy X-ray absorption assay (DXA) and | | | | | | | | | | | |
| (BMD) of the lumbar | | | examined every 3 months after treatment. | | | | | | | | | | | |
| spine, hip, and femur | | | | | | | | | | | | | | |
| and neck | | | | | | | | | | | | | | |
| Brain MRI ^{r} | X | | During the study, brain MRI will be performed upon clinical signs of brain | | | | | | | | | | | |
| | | | metastasis or PET-CT-suggested brain metastases or when necessary as | | | | | | | | | | | |
| | | | judged by the Investigator. | | | | | | | | | | | |
| Chest CT | X | | During the study, the Investigator will perform examinations | | | | | | | | | | | |
| (plain/contrast- | | | according to clinical needs. | | | | | | | | | | | |
| enhanced scan) ^{r} | | | | | | | | | | | | | | |
| MRI scan of lesions ^{r} | X | | Relevant tumor imaging will be performed every 9 weeks (3 cycles). | | | | | | | | | | | |
| PET-CT ^{r} | X | | Additional scans may be performed during the trial if clinical symptoms are found. | | | | | | | | | | | |
| Survival status ^{s} | | | | | | | | | | | | | | X |
| Subsequent anti-tumor | | | | | | | | | | | | | X | X |
| treatment | | | | | | | | | | | | | | |

Abbreviations: ADA = anti-drug antibody; BP = blood pressure; C = cycle; D = day; ECG = electrocardiogram; ECHO = echocardiography; ECOG = Eastern Cooperative Oncology Group; PK = pharmacokinetics; PS = physical status; EOT = end of treatment; EOS = end of study;
*Description of Procedure/Assessment Arrangements:
• Day 1 (D 1) of any cycle is defined as the date on which ALMB-0168 is administered within the given cycle. Procedures/assessments can be arranged within +/−3 days.
• The Day −7 screening/baseline tests and associated assessments can be completed within +/−7 days (from Day −14 to Day −1).
• All pre-dose laboratory tests and other assessments listed for D 1 can be completed within 3 days prior to dosing, except that pre-dose ECG on C1D1 should be completed on C1D1 at the actual dosing visit.
• Visits, laboratory tests and other assessments (excluding PK blood sample collection) listed for D 1 and D 2 of the subsequent cycles can be scheduled within +/−3 days.
• For Days 8 and 15 of cycles 1 and 2, visits or/and laboratory or other assessments are allowed to be completed in a +/−2 days window.
• For all imaging procedures, tumor assessments, and bone mineral density tests, according to the SoA, they can be completed in a +/−5 days window of the specified date.
^{a} The informed consent must be obtained ≤28 days prior to initiation of study treatment.
^{b}Vital signs, including resting pulse, respiration, blood pressure and body temperature, will be measured at screening, on C1D1, C1D8, C1D15, C2D1, C2D8, C2D15, D 1 of each subsequent cycle, and at the EOS visit. It is recommended to take a break for at least 5 minutes, and all examinations should be completed before dosing at that visit.
^{c}Physical examinations, including skin, mucosa, lymph nodes, head (including skull, ears, eyes, nose, mouth, and throat), neck, chest (including thorax, breasts, lungs, and heart), abdomen (including liver, gallbladder, spleen, kidney, bladder, stomach, and intestines), spine/limbs, nervous system, and others, will be performed at screening, on C1D1, C1D8, C1D15, C2D1, C2D8, C2D15, on D 1 of each subsequent cycle and at the end-of-study visit. If the C1D1 physical examination is expected to be done within 72 hours after the baseline physical examination, it can be omitted. These examinations should be completed prior to dosing at that visit.
^{d}Body height: Body height is measured at screening only.
^{e}Body weight: Body weight will be measured during the screening period and on CXD1 (if the body weight examination at screening is completed within 72 h pre-dose, it is not necessary to perform the body weight examination on C1D1.).
^{f}Echocardiography (ECHO): ECHO will be performed within 14 days prior to the initiation of study treatment, and will be performed as needed during the study.
^{g}12-lead ECG: At Screening, 30 minutes (±10 minutes) pre-dose and 30 minutes (±10 minutes) post-dose on C1D1, at any time on C1D8 and C1D15, and 30 minutes (±10 minutes) pre-dose and 30 minutes (±10 minutes) post-dose on D 1 in each subsequent cycle, and at the EOS visit. Subjects will be allowed to rest in the supine position for 5 to 10 minutes before ECG examination, and will remain awake and lying on their back during ECG. Electrocardiogram examination can be added if clinically necessary.
^{h} All medical AEs occurring from signing the ICF to 28 days after the last dose will be collected for Assessment. Specific DLT assessments will be performed from dosing until the end of C1 (C1D21) or prior to the scheduled dosing on C2D1 (i.e. C1D22).
ⁱHematology: Include red blood cell count (RBC), hemoglobin (HGB), hematocrit (HCT), white blood cell count (WBC), platelet count (PLT) and counts and percentages of neutrophils, lymphocytes, eosinophils, basophils and monocytes. will be performed at screening, on C1D1, C1D8, C1D15, C2D1, C2D8, C2D15, on D 1 of each subsequent cycle and at the end-of-study visit. If the C1D1 physical examination is expected to be done within 72 hours after the baseline physical examination, it can be omitted.
^{j}Blood chemistry: Include alanine aminotransferase (ALT), aspartate aminotransferase (AST), alkaline phosphatase (ALP), lactate dehydrogenase (LDH), total bilirubin (TBIL), direct bilirubin (DBIL), total protein (TP), albumin (ALB), urea or blood urea nitrogen (BUN), creatinine (Cr), creatinine clearance (Ccr), glucose (Glu), potassium (K+), sodium (Na+), chloride (Cl−), calcium (Ca2+), magnesium (Mg2+), phosphorus (P), and carbon dioxide. Myocardial zymogram will include creatine kinase (CK) and its isoenzyme (CK-MB). It will be performed at screening, on C1D1, C1D8, C1D15, C2D1, C2D8, C2D15, D 1 of each subsequent cycle, and at the end-of-study visit. If the C1D1 physical examination is expected to be done within 72 hours after the baseline physical examination, it can be omitted.
^{k}Coagulation function: Include prothrombin time (PT), activated partial thromboplastin time (APTT), international normalized ratio (INR), thrombin time (TT), and plasma fibrinogen (Fbg), which will be tested at screening, before dosing on D 1 of each cycle, and at the end-of-study visit. If the time of C1D1 test is within 72 h after the baseline test, it is not necessary to perform the C1D1 test.
^{l}Urinalysis: Include pH, specific gravity (SG), glucose (GLU), protein (PRO), red blood cell (RBC), white blood cell (WBC), ketone (KET), occult blood, bilirubin, nitrite, and urobilinogen. will be performed at screening, on C1D1, C1D8, C1D15, C2D1, C2D8, C2D15, on D 1 of each subsequent cycle and at the end-of-study visit. If the C1D1 physical examination is expected to be done within 72 hours after the baseline physical examination, it can be omitted.
^{m}Serum or urine pregnancy test: The serum pregnancy test will be performed at the baseline test (to be completed within 7 days of the first dose, and it is not necessary to perform the C1D1 test if the time of the baseline test is within 72 h), and the urine pregnancy test can be performed at other time points. For women of childbearing potential, the pregnancy test will be performed before dosing on D 1 of each cycle and at the end-of-study visit.
ⁿVirus serology: Test at baseline. Hepatitis B five-item test (hepatitis B surface antigen (HBsAg), hepatitis B surface antibody (HBsAb), e-antigen (HBeAg), e-antibody (HBeAb) and core antibody (HBcAb); those with positive HBsAg will be further examined for HBV-DNA), hepatitis C antibody (those with positive HCV antibody will be further examined for HCV-RNA), anti-HIV antibody, and anti-treponema pallidum specific antibody;
^{o}PK blood sample collection time: 1. All subjects in the dose-escalation stage and the first 10 subjects in each dose group in the dose-expansion stage will be subject to intensive sampling. Intensive sampling time points: Pre-dose (within 30 minutes) and immediately (within 5 minutes), 2 hours (±15 minutes), 4 hours (±30 minutes), 8 hours (±1 hour), 24 hours (±2 hours), 48 hours (±2 hours), 168 hours (±12 hours), and 336 hours (±24 hours) after the end of infusion on C1D1 and C3D1. Pre-dose (within 30 minutes) and immediately (within 5 minutes) after the end of infusion on D 1 of C2, C4, C5, C7 and every 4 cycles thereafter; at the end of treatment (EOT) and 28 days (+ 3 days) after the final dose. For subjects aged ≥12 years and <16 years, PK samples at 2, 8, and 48 hours after the end of infusion on C1D1 and C3D1 may not be collected. 2. The rest of the subjects in the dose expansion stage will be sparsely sampled. sparse sampling time points: Pre-dose (within 30 minutes) and immediately (within 5 minutes) after the end of infusion on D 1 of C1, C2, C3, C5, C7 and every 4 cycles thereafter; at EOT and 28 days (±3 days) after the final dose. 3. The number and time points of PK sampling may change with the actual condition of the study. In case of infusion reactions or DLT or drug-related SAEs, unplanned PK blood sampling can be performed with laboratory test blood collection at the same visit, if possible.
^{p}Immunogenicity blood sample collection time: Pre-dose (within 30 minutes) on D 1 of C1, C2, C3, C5, C7 and every 4 cycles thereafter, at EOT and 28 days (±3 days) after the final dose. Immunogenicity samples will be used for anti-drug antibody (ADA) testing. In case of infusion reactions or DLT or drug-related SAEs, unplanned immunogenicity blood sampling can be performed with laboratory test blood collection at the same visit, if possible.
^{q} Biomarker samples: All subjects will be required to collect tumor tissue sections or agree to undergo tumor tissue biopsy during screening for IHC testing of biomarkers CD39 and CD73 by the central laboratory (7 to 10 tumor tissue sections will be required), and the test results will be analyzed retrospectively.
^{r} Efficacy will be assessed using RECIST1.1, ICDS, EORTC and PERCIST criteria simultaneously in the study; Systemic PET-CT, lesion MRI, brain MRI and chest CT will be required during screening, and abdominal or pelvic or extremity CT (plain/contrast-enhanced scan) can be added if necessary. During the study, the Investigator may perform brain MRI, chest or abdomen or pelvic cavity or extremities CT (plain/contrast-enhanced scan) according to clinical needs. Refer to the imaging operation manual provided by Independent Review Committee (IRC) for specific imaging examination methods for each site. Additional scans may be performed during the study if clinical suspected symptomatic progression is found. Subjects who discontinued study treatment for any reason other than PD as assessed by both investigator and IRC, death/lost to follow-up, and use of new anti-tumor treatment must continue their imaging assessments by the Investigator or IRC as scheduled (imaging examination once every 9 weeks) until use of new anti-tumor treatment, PD, withdrawal of informed consent, death, or termination of this study, whichever occurred first.
^{s} All subjects will receive an outpatient visit or telephone follow-up every 3 months from the EOT to follow up on subsequent anti-tumor treatment and survival status.

### Response Evaluation

### Tumor Assessment

Imaging procedures in appropriate tumor-associated anatomical areas: CT. MRI contrast-enhanced and/or PET-CT of tumor-related sites, brain CT or MRI.

The results of subjects within 4 weeks prior to dosing are acceptable in the tumor assessments performed during the screening period. During the screening period, the Investigator should select the appropriate tumor assessment method according to the subject's condition, and the tumor assessment during the follow-up period should be conducted in the same method as for the baseline assessment. Once every 9 weeks (3 cycles) during the study. Additional scans may be performed during the study if clinical symptoms are found.

Subjects who discontinued study treatment for any reason other than PD as assessed by both investigator and IRC, death/lost to follow-up, and use of new anti-tumor treatment must continue their imaging assessments by the Investigator or IRC as scheduled (imaging examination once every 9 weeks) until use of new anti-tumor treatment, PD, withdrawal of informed consent, death, or termination of this study, whichever occurred first.

### Endpoint Indicators

Objective Response Rate (ORR): Proportion of subjects with confirmed Complete Response (CR) or Partial Response (PR) from the first dose of the investigational drug until PD (assessed by the Investigator or IRC according to RECIST v1.1, ICDS, EORTC, or PERCIST criteria, respectively), or subsequent anti-tumor treatment, whichever occurs first.

Progression Free Survival (PFS): The time from the first dose of ALMB-0168 to the first documented tumor progression (assessed by the Investigator or IRC according to RECIST v1.1, ICDS, EORTC, or PERCIST criteria, respectively), or death due to any cause, whichever occurs first. [0270]4-month Progression-Free Survival Rate (4m-PFSR): The percentage of subjects who survived without PD within 4 months from the start of ALMB-0168 (as assessed by the Investigator or IRC using RECIST v1.1, ICDS, EORTC, or PERCIST criteria, respectively).

Duration of Response (DoR): The time from the subject's first objective response assessed as Complete Response (CR) or Partial Response (PR) until Progressive Disease or death, whichever occurs first.

Time to Response (TTR): The time from the first dose of ALMB-0168 to the first occurrence of objective response (PR or CR).

Disease Control Rate (DCR): The proportion of subjects with optimal objective response achieving CR, PR or SD.

Overall Survival (OS): The time interval from the first dose of ALMB-0168 to the date of death due to any cause.

Rate of change from baseline in alkaline phosphatase (ALP) or lactate dehydrogenase (LDH).

Skeletal related events (SREs): including changes from baseline in the number of pathological fractures, spinal cord compression, malignancy-associated hypercalcemia, disease-modifying radiotherapies or surgeries for bone lesions, etc.

Change from baseline in the NRS score.

Change from baseline in the EQ-5D score.

Bone mineral density of lumbar spine, hip, and femur and neck: Measured by dual-energy X-ray absorption assay (DXA), with changes from baseline collected.

Change from baseline in dose and frequency of morphine use.

The time points for evaluation of efficacy indicators including ALP, LDH, SRE, NRS, EQ-5D, bone mineral density and morphine use are detailed in the SoA table

### Response Criteria

The Investigator and IRC will perform imaging assessments according to RECIST v1.1, ICDS, EORTC or PERCIST respectively during screening and each tumor assessment cycle. The same imaging technique as baseline will be used throughout the study. If the subject's tumor-related symptoms are aggravated during treatment, tumor assessment can be performed in advance as judged by the Investigator.

Drug therapy may be discontinued for those with progressive disease shown by imaging procedures.

In rare cases, if the Investigator believes that the treatment benefits the subject, the subject with radiographically confirmed PD may also continue the study treatment. It is required to consult the Sponsor first.

Skeletal related events (SREs), dose and frequency of morphine use, NRS, and EQ-5D scores should be collected at the respective visits (see SOA for details).

Pharmacokinetic, Immunogenicity and Biomarker Assessment Pharmacokinetic Assessment

Blood samples for PK will be collected from the contralateral arm at the following time points as specified in the SoA. The number and time points of PK sampling may change with the actual condition of the study. In case of infusion reactions or drug-related SAEs, unplanned PK blood sampling can be performed with laboratory test blood collection at the same visit, if possible. PK samples will be contracted to Beijing Yingji Pharmaceutical Cold Chain Technology Co., Ltd. for transportation, for specific information on the collection, processing, storage and transportation, please refer to the laboratory manual

Intensive sampling will be performed for all subjects in the dose-escalation stage and the first 10 subjects in each dose group in the dose-expansion stage. Intensive sampling time points: Pre-dose (within 30 minutes) and immediately (within 5 minutes), 2 hours (±15 minutes), 4 hours (±30 minutes), 8 hours (±1 hour), 24 hours (±2 hours), 48 hours (±2 hours), 168 hours (±12 hours), and 336 hours (±24 hours) after the end of infusion on C1D1 and C3D1. Pre-dose (within 30 minutes) and immediately (within 5 minutes) after the end of infusion on D1 of C2, C4, C5, C7 and every 4 cycles thereafter; at the end of treatment (EOT) and 28 days (±3 days) after the final dose. For subjects aged ≥12 years and <16 years, PK samples at 2, 8, and 48 hours after the end of infusion on C1D1 and C3D1 may not be collected. The pharmacokinetic parameters of subjects with PK intensive sampling include Cₘₐₓ, Tₘₐₓ, AUC₀₋ₜ, C_{trough}, as well as CL, V_{z}, Vₛₛ, λ_{z}, t_{1/2} and AUC_{0-inf}.

The rest of the subjects in the dose expansion stage will be sparsely sampled. sparse sampling time points: Pre-dose (within 30 minutes) and immediately (within 5 minutes) after the end of infusion on D1 of C1, C2, C3, C5, C7 and every 4 cycles thereafter; at EOT and 28 days (±3 days) after the final dose.

The serum concentration of ALMB-0168 will be tested using a validated analytical method, and PK samples may also be used to evaluate the subject matrix selectivity and target interference of the PK method.

### Immunogenicity Evaluation

Blood samples for the determination of anti-ALMB-0168 antibodies (ADA) will be collected from the contralateral arm at the time points specified in the SoA. In case of infusion reactions or drug-related SAEs, unplanned immunogenicity blood sampling can be performed with laboratory test blood collection at the same visit, if possible. Immunogenicity samples will be contracted to Beijing Yingji Pharmaceutical Cold Chain Technology Co., Ltd. for transportation, for specific information on the collection, processing, storage and transportation, please refer to the laboratory manual.

Samples from subjects to analyze immunogenicity/drug concentrations may also be used to assess safety or efficacy, to address issues that arise during or after the study, or to assist the Sponsor or Sponsor's designee in evaluating relevant biomarkers.

Immunogenicity blood will be collected from all subjects at the following sampling points: Pre-dose (within 30 minutes) on D1 of C1, C2, C3, C5, C7 and every 4 cycles thereafter, at EOT and 28 days (±3 days) after the final dose.

The immunogenicity of ALMB-0168 will be tested adopting a validated analytical method. The immunogenicity samples may be used for in-study cutpoint delimitation, anti-drug antibody analysis, target interference study, etc. in clinical studies.

CD39 and CD73 immunohistochemistry (IHC) test: All subjects will be required to collect tumor tissue sections or agree to undergo tumor tissue biopsy during screening for IHC testing of biomarkers CD39 and CD73 by the central laboratory (7 to 10 tumor tissue sections will be required), and the test results will be analyzed retrospectively. Biomarkers will be contracted to OrigiMed (Shanghai) Co., Ltd. for testing. See the laboratory manual for specific information on the collection, processing, storage and transportation

### Statistical Considerations

There are no formal statistical hypotheses for this study and the statistical analysis results are exploratory.

### Sample Size Estimation

Phase I Part I (dose-escalation stage): An accelerated titration and 3+3 design will be used for the dose-escalation stage. The first two dose groups will be accelerated titration groups, with 1 subject enrolled. The “3+3” dose escalation will be started from the 3rd dose group, with 3 or 6 subjects enrolled in each dose group. The sample size in the dose escalation stage should be no more than 48 subjects.

Phase I Part II (dose-expansion phase): up to 180 subjects will be enrolled.

### Dose Escalation Analysis Population

Efficacy-Evaluable Set (EES): It includes all subjects who have received at least one dose of ALMB-0168 with at least one post-dose efficacy evaluation available.

Safety Analysis Set (SS): it includes all the subjects who have received at least one dose of ALMB-0168. SS is used for safety and tolerability analyses. In the safety analysis, all the subjects will be analyzed by the drug actually received.

Therefore, the SS is defined as consistent with the Full Analysis Set (FAS).

Pharmacokinetic Concentration Set (PKCS): It includes all subjects who have received at least one dose of ALMB-0168 and have at least one evaluable plasma concentration data.

Pharmacokinetic Parameter Set (PKPS): It includes all subjects who have received at least one dose of ALMB-0168 and have at least one evaluable PK parameter.

Immunogenicity Set (IS): It includes all subjects who have received at least one dose of ALMB-0168 and have baseline and at least one post-baseline immunogenicity evaluable data.

Biomarker Analysis Set: It includes all the enrolled subjects who have received at least one dose of the study drug and have at least one biomarker data.

### Statistical Analysis

Statistical analyses are completed using SAS 9.4 or above version.

Descriptive analysis: Continuous data are described by the number of cases, mean, standard deviation, median, quartile, minimum and maximum; categorical data will be analyzed using the number of cases and percent. The minimum and maximum values will be rounded to the same number of decimal places as the raw data. The mean, median and quartile will be rounded to one more decimal place than the raw data. The standard deviation will be rounded to two more decimal places than the raw data. The upper and lower limits of the confidence interval will be reserved with one more decimal place than the point estimate. Percentages will be presented to two decimal places. Usually, up to four decimal places will be retained in statistical tables. Unless otherwise specified, if the decimal place of the original data exceeds two digits and the minimum non-zero value of the absolute value of the original data is ≥0.01, the decimal place of statistical measurement should be two decimal places of the original data.

Baseline is defined as the last non-null value prior to dosing. Unscheduled visits will not be included in the summary analysis by visit. Unless otherwise stated, the analyses will be performed by groups.

Summarize randomization, medication, exclusion, and dataset division of subjects and use the subject distribution flow chart for presentation. A detailed list of subject disposition will be provided.

### Pharmacokinetics, Immunogenicity and Biomarker Analysis

### 1. Pharmacokinetic Analysis

Plasma concentration analysis: Based on PKCS, plasma concentration data will be summarized and tabulated with descriptive statistics by visit and scheduled sampling time point by dose group, and the individual, mean and median plasma concentration-time curves (linear and semi-log scale scales) will be plotted respectively.

PK parameter analysis: Based on PKPS, the PK parameters of each subject will be calculated by non-compartmental model, including Cmax, Tmax, Ctrough, CL, Vz, Vss, λz, t½, AUC0-t, and AUC0-inf (for intensively sampled patients only). Based on the PKPS, individual PK parameters of subjects will be listed and descriptive statistical analysis (including arithmetic and geometric means, median, range, standard deviation, coefficient of variation, geometric coefficient of variation, etc.) will be performed for PK parameters. Box-whisker plots of dose-normalized primary PK parameters (e.g., AUC and Cmax) will be prepared, and the relationship between primary PK parameters and dose will be explored using the ANCOVA model and/or Power model, as applicable.

### 2. Immunogenicity Analysis:

All ADA results will be tabulated based on the IS. Analyze the proportion and ADA titer of ADA-positive subjects as well as the time and duration of the first onset of ADA-positive of subjects. The impact of immunogenicity on drug PK, safety and efficacy will be explored if applicable.

### 3. Biomarker Analysis:

The correlation between the expression of CD39 and CD73 proteins in tumor cells, immune cells and tumor microvascular will be analyzed.

### Safety Analysis

Safety analysis is based on SS analysis. Drug exposure will be summarized as treatment duration, number of dose and dose intensity.

### 1. Adverse Event.

All the adverse events will be coded with MedDRA. Only treatment-emergent adverse event (TEAE) will be analyzed, i.e., adverse events that occur after the first administration of ALMB-0168; Non-TEAE events will not be included in the analysis, with a simple list provided; if the start date/time of the adverse event is incomplete, judge whether it is TEAE according to the available partial information; if the adverse event cannot be judged due to the missing of start date/time, it will be considered a TEAE. Unless otherwise specified, AEs refer to TEAE.

Provide the case number and percent of the subjects with adverse events, drug-related adverse events, serious adverse events, serious adverse events related to ALMB-0168, and the case number and percent of the subjects by System Organ Class (SOC) and Preferred Term (PT); also provide the case number and percent of the subjects by SOC, PT and severity for the adverse events and drug-related adverse events. Data listings will be provided for the AEs resulting in death.

### 2. Laboratory Test.

Provide cross tables of clinical significance and baseline for each visit and summary tables of clinically significant new abnormalities (considering unscheduled visits) in laboratory tests.

### 3. Vital Signs.

The observed values and changes from baseline of vital signs (temperature, pulse, respiration, systolic/diastolic blood pressure) for each visit should be tabulated by the number of cases, mean, standard deviation, median, minimum and maximum.

### 4. ECG.

The observed values and changes from baseline in ECG examinations (heart rate, PR interval, QRS duration, QT interval, QTc interval) for each visit should be tabulated by the number of cases, mean, standard deviation, median, minimum and maximum. Provide cross tables of clinical significance and baseline for each visit and summary tables of clinically significant new abnormalities (considering unscheduled visits).

### 5. Death

Deaths reported during the study treatment period (within the TEAE window) and deaths reported during the follow-up period will be summarized and analyzed.

### Efficacy Analysis

No formal efficacy analysis will be performed during the dose escalation stage, and a descriptive summary of efficacy will be performed in the efficacy evaluable set if required. The BOR, ORR and DCR will be summarized and analyzed at the dose expansion and cohort expansion stages. If supported by the data, progression-free survival (PFS), duration of response (DoR), time to response (TTR), 4-month progression-free survival rate (4m-PFSR), 6-month progression-free survival rate (6m-PFSR), and overall survival (OS) will also be analyzed. The method of analysis is outlined as below:

BOR will be summarized by cohort according to RECIST v1.1, ICDS, EORTC and PERCIST criteria, the number and percentage of subjects with objective response rate (ORR), disease control rate (DCR) will be calculated, and the 95% confidence interval (CI) will be calculated using Clopper-Pearson exact method.

If data are applicable, the PFS, DoR and OS will be analyzed by Kaplan-Meier method, including estimation of the median time and its 95% CI, and calculation of the PFSR at 4, 6, 8, 12, . . . months and their 95% CIs. Censoring rates, quartiles and Kaplan-Meier curves will be presented separately. A descriptive analysis of the TTR will be performed if data are applicable.

Rate of change from baseline in alkaline phosphatase (ALP) or lactate dehydrogenase (LDH); skeletal related events (SREs); changes from baseline in Numeric Rating Scale (NRS) and Europe Health-related Quality of Life Questionnaire 5 Dimensions (EQ-5D) scores; bone mineral density (BMD) of the lumbar spine, hip, femur, and neck; dose and frequency of morphine use will be provided.

### Appendices

### Appendix I: ECOG Physical Performance

The ECOG Performance Status Scale is shown as in Table 13 below.

TABLE 13
ECOG Performance Status Scale

| Grade | Physical Status |
|---|---|
| 0 | Fully active, able to carry on all pre-disease performance without restriction. |
| 1 | Restricted in physically strenuous activity, but ambulatory and able to carry out |
| | work of a light or sedentary nature, such as light house work or office work. |
| 2 | Ambulatory and capable of all self-care, but unable to carry out any work |
| | activities; up and about more than half of waking hours. |
| 3 | Capable of only limited self-care; confined to bed/chair more than half of waking |
| | hours. |
| 4 | Completely disabled; cannot carry out any self-care; totally confined to bed/chair. |
| 5 | Death |

### Appendix II. Cardiac Function Classification of New York Heart Association (NYHA)

The Cardiac Function Classification of New York Heart Association is shown in Table 14.

TABLE 14
NYHA Cardiac Function Classification.

| Grade | Symptoms |
|---|---|
| I | The patient has cardiac organic disease, but routine activity is not restricted, and |
| | general activity does not cause fatigue, palpitation, dyspnea, or angina. |
| II | The physical activity of the patient is slightly restricted, and there is no subjective |
| | symptom during rest, but fatigue, palpitation, dyspnea or angina may occur during |
| | general activities. |
| III | The physical activity of the patient is obviously limited, and general activities |
| | lighter than usual cause fatigue, palpitation, dyspnea or angina. |
| IV | The patient cannot engage in any physical activity. At rest, there are also |
| | symptoms of cardiac insufficiency, which are aggravated after physical activity. |

### Appendix III. Excerpt of Response Evaluation Criteria in Solid Tumors (RECIST 1.1)

### Baseline Tumor Measurement

At baseline, tumor lesions/lymph nodes are classified as measurable and non-measurable, as below.

### 1. Measurable Lesions

Tumorous lesion: tumor lesion. The tumor lesion has at least one diameter that can be accurately measured (recorded as the longest diameter of the measurement plane), and its minimum length is as follows:
10 mm for computed tomography (CT) (CT slice thickness not greater than 5 mm).
10 mm measured by calipers for clinical examination (lesions that cannot be accurately measured by calipers should be recorded as non-measurable).
20 mm for chest X-ray.

Malignant lymph nodes: When evaluated by CT (CT slice thickness is recommended to be not greater than 5 mm), the minor axis of the lymph node must reach 15 mm to be considered pathologically enlarged and measurable. Only minor axis lengths are measured and followed up for pre-operation and follow-up.

### 2. Non-Measurable Lesions

All other lesions, including small lesions (with a maximum diameter of less than 10 mm or the minor axis of pathological lymph node of 10 mm to less than 15 mm) and truly non-measurable lesions. Lesions considered to be truly measurable include pia mater lesions, ascites, pleural or pericardial effusion, inflammatory breast disease, lymph of skin/lung infiltration, peritoneal dissemination, as well as abdominal mass/abdominal organ enlargement that cannot be confirmed by repeated imaging techniques and physical examination.

### 3. Special Considerations Regarding Measurable Lesions

Bone lesions, cystic lesions, and lesions that have been locally treated should be specifically noted, as outlined below: Bone lesions:
Bone scans, positron emission tomography (PET) scans, or plain radiographs are not considered appropriate imaging techniques for measuring bone lesions. However, these techniques can be used to confirm the existence or disappearance of bone lesions.
For osteolytic lesions or mixed osteolytic/osteogenic lesions with a defined soft tissue component, they can be evaluated as measurable lesions with tomographic imaging techniques such as CT or MRI, provided the soft tissue component meets the above definition of measurability.
Osteogenic lesions are non-measurable. Cystic lesions:
Lesions that meet the criteria for the definition of simple cysts on radiologic imaging should not be considered malignant (neither measurable nor non-measurable) because they are defined as simple cysts.
If cystic lesions represent cystic metastatic lesions and meet the measurability defined above, they can be considered measurable. However, if non-cystic lesions are present in the same patient, non-cystic lesions should be preferred as target lesions.

Lesions that have been locally treated:
A tumor lesion located in a previously irradiated area or at a site subjected to other local regional treatments is generally considered a non-measurable lesion, unless it shows unequivocal progression. The study protocol should detail the conditions under which such lesions would be considered measurable.

### Measurement Method Specification

### 1. Measurement of Lesions

All measurements should be recorded in metric notation, using calipers if clinically evaluated. All baseline evaluations must be performed as close to the start of treatment as possible, and less than four weeks earlier.

### 2. Measurement Methods

The same determination methods and techniques should be used to describe each identified and reported lesion at the baseline and follow-up phases. Normally, imaging examinations (instead of clinical examinations) should be carried out, unless the lesion is found to be unsuitable for imaging at follow-up.

Clinical lesion: Only superficial lesions (such as subcutaneous nodes) with a caliper measurement of 10 mm or more in diameter are considered measurable. For cases of skin lesions, it is recommended to use color photographs with the proportion of lesion size measured. As mentioned earlier, when the lesion can be examined both clinically and by imaging, imaging should be used, because the imaging evaluation is more objective and can be used for the final review of clinical studies

Chest X-ray examination: Compared with chest X-ray examination, chest CT is preferred, especially when progressive disease is used as an important endpoint, because CT scans are more sensitive than X-ray examinations in identifying new lesions. However, if the X-ray reveals a clear lesion boundary, which is surrounded by an inflated lung, it is considered measurable.

CT, MRI: CT is currently the most effective and repeatable test method to evaluate the response of lesions. As defined by the guideline, CT slice thickness should be no more than 5 mm for measurable lesions. When the CT slice thickness exceeds 5 mm, the minimum measurable lesion should be twice the slice thickness. MRI can also be used under certain circumstances (such as whole body scan).

Ultrasonography: Ultrasonography is not suitable for evaluating lesion size and should not be applied as a measurement method. Ultrasonography cannot be completely reproduced between two adjacent observations. Moreover, the results depend on the examiner. Technique and measuring results cannot be secured the same for one test and its next. If new lesions are detected by ultrasonography during the course of the study, CT or MRI validation is recommended. If CT radiation exposure is a concern, MRI can be used instead to detect the lesion to be examined.

Endoscopy, laparoscopy: These techniques are not recommended for objective tumor evaluation. However, they are beneficial in confirming complete pathological response with biopsy or in determining complete response or recurrence after surgical resection.

Tumor markers: tumor markers cannot be used alone to evaluate the objective response of the tumor. However, when the tumor markers begin to rise above the upper limit of normal, the markers must be reduced to the normal range if used to determine the complete response of the patient. Because tumor markers are disease-specific, indications for testing for a particular disease should be included in the protocol. Specific guidelines on CA-125 changes (ovarian cancer recurrence) and PSA changes (prostate cancer recurrence) have been published. In addition, the Gynecologic Cancer Intergroup developed the CA-125 progression criteria for clinical studies with objective tumor evaluation indicators for first-line treatment of ovarian cancer.

Cytology, histology: These techniques can often be used to distinguish between partial and complete responses in individual cases (e.g., residual benign tumor lesions in the tumor type of reproductive cell tumor) if required by the clinical study protocol. When exudation is known to be a potential adverse consequence of therapy (e.g., certain paclitaxel-based chemotherapeutic agents or angiogenesis inhibitors), measurable tumor lesions that meet the criteria for response or stable disease require cytologic confirmation of oncologic exudation that occurs or worsens during therapy, in order to distinguish between response (or stable disease) and progressive disease.

### Response Evaluation of Tumor

### 1. Evaluation of All Tumors and Measurable Lesions

In order to evaluate objective response or possible future progression, it is necessary to perform a baseline evaluation of the total burden of tumors in all tumor lesions, as a reference for subsequent measurements. In the clinical protocol with objective response as the main treatment endpoint, only patients with measurable lesions at baseline will be enrolled. Measurable lesions are defined as the presence of at least one measurable lesion. For studies with progressive disease (time to progression or degree of progression on a fixed date) as the main treatment endpoint, it must be clear from the protocol inclusion criteria whether it is limited to patients with measurable lesions or whether patients without measurable lesions can also be enrolled.

### 2. Baseline Records of Target and Non-target Lesions

When more than one measurable lesions are present at baseline evaluation, all lesions should be recorded and measured, totaling no more than 5 (no more than 2 per organ), as target lesion representing all involved organs (i.e., patients with only one or two cumulative organs should take a maximum of two or four target lesions as baseline measurement lesions).

Target lesions must be selected based on lesion size (maximum diameter), representative of all involved organs, and measurements must be repeatable. In the case that the largest lesion cannot be measured repeatedly, another largest lesion which can be measured repeatedly.

Lymph nodes can still be imaged in normal anatomy without tumor metastasis and therefore require special attention. Pathological lymph nodes defined as measurable nodules or even target lesions must meet the following criteria: short diameter ≥15 mm measured by CT. Only short diameter needs to be detected at baseline. Radiologists usually use the short diameter of a nodule to determine whether there is tumor metastasis. Nodule size is generally expressed by two-dimensional data of imaging examinations (axial plane is commonly used for CT scanning, and axial plane, sagittal plane or coronal plane is selected for MRI). The short diameter is determined by the minimum value taken. For example, a 20 mm×30 mm abdominal nodule with a short-axis diameter of 20 mm can be considered a malignant, measurable nodule. In this example, 20 mm is the measurement of the nodule. Nodules ≥10 mm but <15 mm in diameter should not be considered target lesions. Nodules <10 mm are not considered pathological and should not be recorded or further observed.

The calculated sum of the diameters of all target lesions (including the maximum diameter of non-nodular lesion and the short-axis diameter of nodular lesion) will be the sum of the baseline diameters. If the diameter of lymph nodes is involved, as noted above, only the short diameter will be included. The sum of baseline diameters will be used as a reference for baseline disease.

All remaining lesions including pathological lymph nodes should be considered non-target lesions without measurement but should be recorded at baseline evaluation as follows: “Present,” “Absent” or, in a few cases, “Unequivocal Progression.” Extensive non-target lesions in the same organ may be recorded on the case report form as a single item, such as numerous enlarged pelvic lymph nodes or extensive liver metastases.

### 3. Response Evaluation Criteria

### 3.1. Response Evaluation on Target Lesions

Complete response (CR): All target lesions are absent, and the short-axis diameter of any pathological lymph node (whether it is a target lesion or not) must be <10 mm.

Partial response (PR): The sum of the diameters of all target lesions is reduced by at least 30% relative to the sum of baseline diameters.

Progressive disease (PD): The sum of the diameters of all target lesions is increased by at least 20% relative to the sum of the diameters of the minimum lesion during the study (including the sum of baseline diameters of lesion if it is the minimum). In addition to the relative increase of 20%, the sum of the diameters must also demonstrate an absolute increase of at least 5 mm (note: the presence of one or more new lesions can also be considered progressive).

Stable disease (SD): Based on the sum of the diameters of the minimum lesion during the study, the lesion reduction is not a PR, and the lesion increase is not a PD.

### 3.2. Considerations for Response Evaluation on Target Lesions

When target lesions are present in lymph nodes:

The measured short-axis diameter should be recorded (at the same anatomic surface as the baseline measurement), even if the lymph nodes are resolved to below 10 mm in the study.

This means that when target lesions are present in lymph nodes, the sum of the diameters of target lesions will not be 0 even if complete response criteria are met, since a lymph node with a short-axis diameter <10 mm is considered normal. Case report forms or other methods of data collection may be designed to have nodular target lesions recorded separately. For complete response, each node must have a short-axis diameter <10 mm. For PR, SD, and PD, the sum of the diameters of target lesions will include the actual short-axis diameters of nodes.

When target lesions are too small to be measured:

For all lesions (nodular and non-nodular) recorded at baseline in the study, their actual measurements must be recorded in subsequent evaluations, even if the lesion is very small (e.g., 2 mm). However, some lesions or lymph nodes recorded as target lesions at baseline have too weak signals on CT scans to be exactly measured by the radiologist, and they can be reported as too small to be measured. In this case, it is important to record one measurement in the case report form. If it is the opinion of the radiologist that the lesion may disappear, the measurement can be recorded as 0 mm. If the lesion does exist with a weak signal, a default value of 5 mm should be assigned (Note: This rule is not applicable to normal lymph nodes since they usually have a definable size and are frequently surrounded by fat such as in the retroperitoneum; however, if the lymph node does exist with a too weak signal to be measured, a default value of 5 mm should also be assigned). The default value of 5 mm is based on the thickness of the CT tomography scan (the default value of 5 mm will not change with this thickness). The measurement of such lesions (too small to be measured) may lack repeatability and should be assigned a default value to prevent evaluation as a false response or false progression due to measurement error. Again, if the radiologist is able to provide an actual measurement, even less than 5 mm, it should be recorded.

When lesions split or coalesce on treatment:

When non-nodular lesions are separated, the maximum diameters of all fragments must be summed for the sum of diameters of target lesions. Similarly, when lesions coalesce, the long-axis diameters can be retained, which helps to obtain the maximum diameter of each individual lesion prior to coalescence. If the lesions have completely coalesced such that they are no longer separable, the vector of the maximum diameter in this instance should be the maximum diameter of the coalesced lesion.

### 3.3. Evaluation of Non-target Lesions

This section defines the criteria for response to non-target lesions tumors. While some non-target lesions are actually measurable, they need not be measured and only need to be qualitatively evaluated at the time points specified in the protocol.

Complete Response (CR): All non-target lesions are resolved and tumor markers return to normal. All lymph nodes are non-pathological in size (short diameter <10 mm).

Non-complete response/non-progressive disease (non-CR or non-PD): There are one or more non-target lesions and/or the tumor marker is constantly above the normal level.

Progressive Disease (PD): Existing non-target lesions show unequivocal progression. Note: The presence of one or more new lesions is also considered a progressive disease.

### 3.4. Considerations for Evaluation of Progression of Non-target Lesions

The definition of progression of non-target lesions is additionally explained below:

When patients have measurable lesions: In this case, for an unequivocal progression of non-target lesions, the non-target lesions are worsened to such a degree that the treatment must be discontinued. Even if the target lesions are evaluated as stable disease or partial response, the overall tumor burden has increased sufficiently to support discontinuation of treatment. Mild increases in one or more non-target lesions are usually not sufficient to be considered as unequivocal progression. In the case of stable disease or partial response of target lesions, it is extremely rare for overall tumor progression to be defined solely by changes in non-target lesions.

When none of the non-target lesions in the patient is measurable: This occurs when the inclusion criteria do not specify that a measurable lesion must be present in some phase III studies. The overall evaluation is still based on the above criteria, but there are no measurable data for lesions in this case. Worsening of non-target lesions is not easy to evaluate (by definition: All lesions are truly non-measurable). Therefore, when changes in non-target lesions lead to an increase in the overall disease burden equivalent to the occurrence of progressive disease in the target lesions, an unequivocal progression should be defined based on non-target lesions, and an effective test should be established for evaluation. For example, an increase in tumor burden represents an additional 73% increase in volume (which is equivalent to a 20% increase in the diameter of measurable lesions). Another example is peritoneal exudation from “minimal” to “large”; lymphatic lesions ranged from “local” to “widespread”; or “change in the treatment method based on sufficient evidence” described in the protocol. In case of an unequivocal progression, the patient should be considered generally to have had progressive disease at that time point. Although it is ideal to have non-measurable lesions evaluated by objective criteria, there may be lesions that are not objectively evaluable in nature and therefore must be significantly enlarged.

### 3.5. New Lesions

The presence of new malignant lesions denotes progressive disease; therefore, some comments on new lesions are important. There are no specific criteria for the identification of new lesions by imaging; however, the finding of a new lesion should be unequivocal. For example, progression cannot be attributed to differences in imaging techniques, changes in imaging morphology, or other lesions other than tumors (e.g., some so-called new bone lesions are just the cured original lesions or the recurrent original lesions). It is important when the patient's baseline lesions show partial or complete response. For example, necrosis of a liver lesion may be documented on a CT report as a new cystic lesion, while it is actually not.

Lesions that are detected at follow-up and are not identified at baseline will be considered new, suggesting progressive disease. For example, in a patient with a visceral lesion identified at baseline, when metastasis is found on CT or MRI of the head, the intracranial metastasis will be considered a basis for progressive disease, even if he does not undergo a head examination at baseline.

If a new lesion is equivocal for its small size, continued therapy and follow-up evaluation are required to confirm whether it is a new lesion. If repeated examinations confirm that it is definitely a new lesion, the time to progression should be calculated from the time it is first discovered.

FDG-PET assessment generally requires additional examination for confirmation, so it is reasonable to combine FDG-PET and CT findings to evaluate the progression (especially for new suspected diseases). New lesions can be identified by FDG-PET examination according to the following procedures:

A negative FDG-PET result at baseline and a positive FDG-PET result at follow-up suggest progressive disease.

No FDG-PET at baseline and a positive FDG-PET result at follow-up:

If the new lesion identified by a positive FDG-PET at follow-up is confirmed by CT, there will be a progressive disease.

If the new lesion identified by a positive FDG-PET at follow-up is not confirmed by CT, additional CT will be needed for confirmation (if confirmed, the time to progression should be calculated from the time when the abnormality is found in early FDG-PET).

If the positive FDG-PET at follow-up confirms an existing lesion identified by CT and the lesion is not progressing on imaging, there will be no progressive disease.

### 4. Evaluation of Best Overall Response

The evaluation of best overall response is a record of the best response from the start of the study to the end of the study, taking into account any necessary conditions for confirmation. On occasion, the response will not be confirmed until the end of treatment, so the protocol should specify whether the response evaluation at the end of treatment is included in the evaluation of best overall response. The protocol must specify how any new treatment that precedes progression affects the best response. The best response of patients depends primarily on the results of target and non-target lesions and the presence or absence of new lesions, as well as the nature of the study, protocol requirements, and outcome measures. Specifically, in non-randomized studies where the response is the primary endpoint, confirmation of PR or CR is a must to determine which is the best overall response.

4.1. It is assumed that response evaluation will be conducted at each specific time point of the protocol. Table 1 provides a summary of the overall response at each time point for patients with measurable disease at baseline.

If the patient has no measurable lesion (no target lesions), the evaluation can be found in Table 2.

### 4.2. Description on Missing Evaluation and Non-evaluable Cases

If no lesion imaging/measurement is done at a specific time point, the patient will be non-evaluable at that time point. If only partial lesions can be evaluated at one time, usually the case will be non-evaluable at that time point, unless a convincing argument can be made that the missing lesions will not affect the response evaluation at the specified time point. This is likely to occur in the setting of progressive disease. For example, if a patient had 3 lesions with a sum of 50 mm in diameter at baseline and only 2 lesions are evaluable later with a sum of 80 mm, the patient will be evaluated as to have progressive disease, regardless of the effect of missing lesions.

### 4.3. Best Overall Response: All Time Points

The best overall response can be determined if all information about the patient is available.

Evaluation of the best overall response when the confirmation of complete or partial response is not required in the study: The best response in the study is the best response at all time points. For example, if a patient has SD in the first cycle, PR in the second cycle, and PD in the last cycle, the best overall response will be PR. When the best overall response is SD, it must also meet the protocol-specified minimum time from baseline. If the minimum time criterion is not met, the best overall response of SD will not be accepted, and it will be determined by the subsequent evaluation. For example, if a patient has SD in the first cycle and PD in the second cycle but the minimum time for SD is not met, the best overall response will be PD. Similarly, the patient lost to follow-up after being evaluated as SD in the first cycle will be considered as non-evaluable.

Evaluation of the best overall response when the confirmation of complete or partial response is required in the study: A complete or partial response can be confirmed only if each subject meets the criteria for a partial or complete response specified in the study and if the response is confirmed at a subsequent time point as specified in the protocol (generally 4 weeks later). In this case, the description of the best overall response is provided in Table 17.

### 4.4. Special Notes on Response Evaluation

When nodular lesions are included in the overall evaluation of target lesions, and the nodules decrease to “normal” size (<10 mm), there will still be scan reports on lesion size. To avoid over-evaluation, measurements will be recorded even if the nodules are normal as reflected by an increase in nodule size. As previously mentioned, this means that 0 will not be recorded on the CRF for the subjects with complete response.

If response confirmation is required during the study, repeated “non-measurable” time points will complicate the best response evaluation. The analysis plan for the study must address how responses will be judged by these missing data. For example, in most studies, the response of PR-NE-PR of a subject can be considered confirmed.

Symptomatic progression should be reported when the subject has a global deterioration requiring discontinuation of treatment without objective evidence. Objective progression should be evaluated as far as possible even after discontinuation of treatment. Symptomatic deterioration is not a descriptor of objective response, but the reason for discontinuation of treatment. The objective response of such subjects will be evaluated by the description of target and non-target lesions as shown in Tables 15 to 17.

Cases defined as early progression, early death and non-evaluable are exceptions of the study and should be clearly described in each protocol (depending on treatment interval and treatment cycle).

In some cases, it may be difficult to distinguish a local lesion from normal tissue. When the evaluation of a complete response is based on such a definition, we recommend that the biopsy be performed prior to the response evaluation of the local lesion complete response. When the abnormal imaging results of local lesions in some subjects are considered to represent the fibrosis or scar formation of the lesion, FDG-PET is used as the evaluation standard similar to biopsy to confirm the complete response. The use of FDG-PET, in this case, should be prospectively described in the protocol, supported by reports from the specialized medical literature on this case. However, it must be acknowledged that both FDG-PET and biopsy may lead to false positive results at the evaluation of complete response due to their own limitations (including resolution/sensitivity).

TABLE 15
Time-Point Response: Subjects with Target Lesions
(With or without Non-target Lesions).

| | | | Overall |
|---|---|---|---|
| Target Lesions | Non-target Lesions | New Lesions | Response |
| CR | CR | None | CR |
| CR | Non-CR/Non-PD | None | PR |
| CR | Non-evaluable | None | PR |
| PR | Non-progressive or non- | None | PR |
| | fully evaluable | | |
| SD | Non-progressive or non- | None | SD |
| | fully evaluable | | |
| Non-fully evaluable | Non-progressive | None | NE |
| PD | Any cases | Yes or No | PD |
| Any cases | PD | Yes or No | PD |
| Any cases | Any cases | Yes | PD |

CR = complete response
PR = partial response
SD = stable disease
PD = progressive disease
NE = Non-evaluable

In SD cases, at least one post-enrollment follow-up measurement must meet SD criteria and the follow-up and enrollment should be separated by at least 6-8 weeks.

TABLE 16
Time-point Response - Subjects with Non-target Lesions Only.

| Non-target Lesions | New Lesions | Overall Response |
|---|---|---|
| CR | None | CR |
| Non-CR or non-PD | None | Non-CR or non-PD |
| Non-fully evaluable | None | Non-evaluable |
| Equivocal PD | Yes or No | PD |
| Any cases | Yes | PD |

Note:
For non-target lesions, “non-CR/non-PD” refers to response superior to SD. Since SD is increasingly used as the endpoint for response evaluation, non-CR/non-PD response is established for cases where no lesion is measurable.

For equivocal findings of progression (e.g., very small and uncertain new lesions; cystic changes or necrosis in existing lesions), treatment may continue until the next evaluation. If in the next evaluation, progressive disease is confirmed, the date of progression should be the date of previously suspected progression.

TABLE 17
Best Overall Response to be Confirmed for CR and PR.

| Overall | Overall | |
|---|---|---|
| Response at | Response at | |
| the First | Subsequent | |
| Time Point | Time Points | Best Overall Response |
| CR | CR | CR |
| CR | PR | SD, PD or PR^{a} |
| CR | SD | If SD persists for enough time, it is SD, |
| | | otherwise, it should be PD |
| CR | PD | If SD persists for enough time, it is SD, |
| | | otherwise, it should be PD |
| CR | NE | If SD persists for enough time, it is SD, |
| | | otherwise, it should be NE |
| PR | CR | PR |
| PR | PR | PR |
| PR | SD | SD |
| PR | PD | If SD persists for enough time, it is SD, |
| | | otherwise, it should be PD |
| PR | NE | If SD persists for enough time, it is SD, |
| | | otherwise, it should be NE |
| NE | NE | NE |

Note:
CR = complete response, PR = partial response, SD = stable disease, PD = progressive disease, NE = non-evaluable. Superscript “a”: If CR actually occurs at the first time point and any disease occurs at subsequent time points, then even if the subject meets the PR criteria relative to baseline, the response will still be PD at subsequent time points (because the disease will reappear after CR). The best response depends on whether SD occurs at the shortest treatment interval. However, sometimes the response is evaluated as CR at the first time point, but scans at subsequent time points suggest a small lesion, so in fact, the response of subjects should be PR rather than CR at the first time point. In this case, the CR evaluated at the first time point should be changed to PR and the best response should be PR.

### 5. Frequency of Tumor Reevaluation

The frequency of tumor reevaluation during treatment depends on the treatment regimen and should be consistent with the type and schedule of treatment. However, in Phase II studies where the benefit of treatment is unclear, follow-up every 6 to 8 weeks (timed to coincide with the end of a cycle) is reasonable, and the length of the interval may be adjusted in special regimens or cases. The protocol should specify which tissue sites are to be evaluated at baseline (usually those most likely to be involved with metastasis of the tumor type under study) and how often evaluations are repeated. Normally, target and non-target lesions are evaluated at each time. In some optional cases, certain non-target lesions may be evaluated less frequently. For example, bone scans may need to be repeated only when CR is identified in target diseases or when progression in bone lesions is suspected.

At the end of treatment, reevaluation of tumors depends on whether the response rate or the time to an event (progression/death) is used as the endpoint of the clinical study. For the time to an event (e.g., TTP/DFS/PFS), the routine repeated evaluation specified in the protocol is required. In randomized comparative studies, in particular, the scheduled evaluations should be outlined in the schedule (e.g., 6 to 8 weeks on treatment or 3 to 4 months after treatment) and should not be affected by delays in treatment, dosing intervals, and any other events that may cause an imbalance in treatment arms in the timing of disease evaluation.

### 6. Response Evaluation/Confirmation of Response Period

### 6.1. Confirmation

In non-randomized clinical studies where the response is the primary endpoint, confirmation of PR and CR is a must to ensure that the response is not the result of an evaluation error. This also allows for a sound interpretation of results in the context of historical data, but the response described in the historical data of these studies should also be confirmed. However, in all other cases, such as randomized studies (phase II or III) or in studies where stable disease or progressive disease is the primary endpoint, response confirmation is no longer required because it is of no value in the interpretation of the study results. Eliminating the need for response confirmation, however, would make a central review that prevents offset even more important, especially in non-blind studies.

In the case of SD, at least one measurement should meet the SD criteria specified in the protocol within the shortest time interval after the study starts (generally not less than 6-8 weeks).

6.2. The overall response period is calculated from the time when CR or PR criteria are met for the first time (whichever is first measured) to the time when the recurrent or progressive disease is objectively documented (the minimum measurement recorded in the study is used as a reference for progressive disease). The overall complete response period is calculated from the time when CR criteria are met for the first time to the time when the recurrent or progressive disease is objectively documented.

### 6.3. Stable Disease Period

It is calculated from the beginning of treatment (in randomized studies, from the time of randomization) to progressive disease, by reference to the sum of the minimum measurements in the study (if the baseline sum is minimum, it is used as a reference for PD calculation). The clinical relevance of the stable disease period varies with studies and diseases. If the proportion of patients achieving stable disease for a minimum period of time is a study endpoint in a particular study, the protocol should specify the minimum time interval between two measurements as defined in the SD.

Note: The response period, stable disease period, and PFS are influenced by the frequency of follow-up after baseline evaluation. It is not within the scope of this guideline to define a standard frequency of follow-up. The frequency of follow-up should be determined based on disease typing and staging., treatment cycle, standard practice, etc. The limitations in the accuracy of the measurement endpoints should be taken into account if comparisons between studies are needed.

### 7. Progression-free Survival (PFS)/Percentage Progression-free (PPF)

This guideline focuses on the use of objective response as a study endpoint in Phase II clinical study. In some cases, the response rate may not be optimal for evaluating the potential anticancer activity of a new drug/regimen. In these cases, PFS/PPF at demarcated time points may be considered a suitable surrogate to provide an initial signal of the biological activity of a new drug. However, it is clear that in an uncontrolled study, these evaluations will be questioned, as seemingly valuable observations may be related to biological factors such as the screening of patients rather than the effect of the drug intervention. Therefore, Phase II clinical studies with these study endpoints are best designed with randomized control. However, since the clinical manifestations of some tumors are consistent (generally, always poor) all the time, the non-randomized studies are reasonable. However, in these cases, due to the lack of positive control, the evidence of efficacy should be recorded with caution when expected PFS or PPF is evaluated.

Note: age is in years and weight is in kilograms (kg).

### Appendix IV: Numeric Rating Scale (NRS) Score

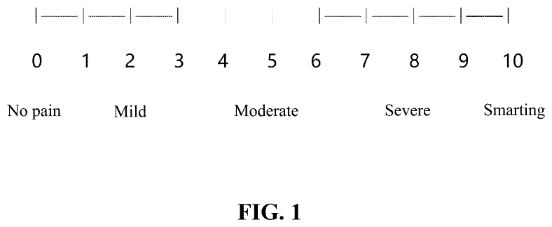

Scores 0-10 represent different degrees of pain. Ask the patient about the degree of pain, and make a mark, or ask the patient to circle the figure that best represents the degree of pain.
0: No pain.
1-3: Mild pain (not affect sleep).
4-6: Moderate pain.
7-9: Severe pain (unable to fall asleep or wake up with pain in sleep).
10: Smarting.
Different degrees of pain is also shown in FIG. 1.

### Appendix V: Europe Health-related Quality of Life Questionnaire 5 Dimensions (EQ-5D)

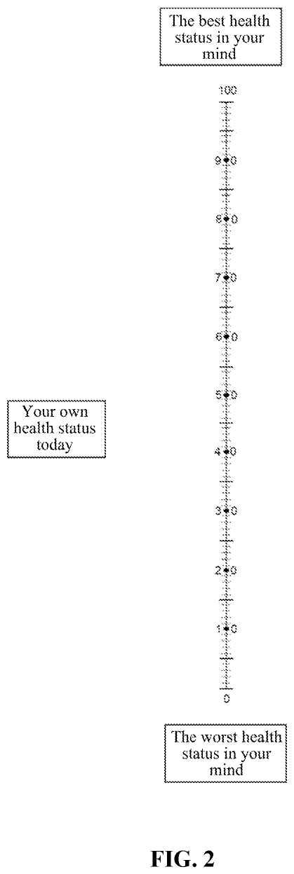

The EQ-5D form is shown below. Patient name, randomized No., date of visit and the corresponding dosing of the visit are documented. The related scale is shown in FIG. 2.

TABLE 18
EQ-5D Questionnaire.
Please select one of the following options (E1-E5) that best reflects your health today:

| | | |
|---|---|---|
| E1 | Action | |
| | (1) I have no problems walking | □ |
| | (2) I have slight problems walking | □ |
| | (3) I am unable to walk | □ |
| E2 | Self-care | |
| | (1) I have no problems taking care of myself | □ |
| | (2) I have slight problems washing, brushing, bathing or dressing myself | □ |
| | (3) I am unable to wash, brush, bath or dress myself. | □ |
| E3 | Usual activities (such as work, study, housework, family or leisure activities) | |
| | (1) I have no problems doing my usual activities | □ |
| | (2) I have slight problems doing my usual activities | □ |
| | (3) I am unable to do my usual activities | □ |
| E4 | Pain/Discomfort | |
| | (1) I have no pain or discomfort | □ |
| | (2) I have moderate pain or discomfort | □ |
| | (3) I have extreme pain or discomfort | □ |
| E5 | Anxiety (nervous, worried, upset, etc.)/Depression (lack of interest, loss of | |
| | fun, loss of spirit, etc.) | |
| | (1) I am not anxious or depressed | □ |
| | (2) I am moderately anxious or depressed | □ |
| | (3) I am extremely anxious or depressed | □ |
| E6 | To reflect your health, we have drawn a thermometer-like scale in which 100 | Results: |
| | represents the best health you can imagine and 0 represents the worst health | |
| | you can imagine. See “VAS Scale” on the back | |

### Appendix VI: ICDS (Modified Inverse Choi Density/Size Criteria) Evaluation Criteria

Modified Inverse Choi Density/Size Criteria are shown in Table 19 below.

TABLE 19
ICDS Evaluation Criteria.

| Complete Response (CR) | All lesions disappeared |
|---|---|
| Partial Response (PR) | ≥10% reduction in size (%Δ Choi SLD) or >15% reduction in CT |
| | density from baseline, no new lesion, and no significant progression |
| | of non-measurable lesions |
| Stable Disease (SD) | Not meeting the criteria for CR, PR, or PD; no symptom worsening |
| | due to tumor progression. |
| Progressive Disease (PD) | ≥10% increase in tumor size (Choi SLD) and CT density not |
| | meeting the criteria for PR; any new lesion on CT/MRI; new |
| | intratumoral nodules or enlargement of existing intratumoral |
| | nodules. |
| Unevaluable (UE): | Unable to receive CT/MRI, or evaluation. If the density and size |
| | measurements show that the target lesion is not evaluable and does |
| | not meet the criteria for PD, the evaluation result is not evaluable. |

Note:
It should be evaluated by contrast-enhanced spiral CT or MRI.
CT attenuation of each tumor should be measured in Hounsfield units (HUs) during the portal venous phase.
SLD stands for sum of the longest diameters.

### Appendix VII. EORTC (European Organisation for Research and Treatment of Cancer) Evaluation Criteria

European Organisation for Research and Treatment of Cancer evaluation criteria is shown in Table 20 below.

TABLE 20
EORTC Evaluation Criteria.

| Reaction | Evaluation |
|---|---|
| CMR | Complete resolution of ¹⁸F-FDG uptake within all lesions and indistinguishable from |
| | surrounding tissues; |
| PMR | Reduction of minimum of 15%-25% in total SUVₘₐₓ uptake of tumor ¹⁸F-FDG after |
| | one course of treatment; reduction of >25% from baseline after more than one course |
| | of treatment; |
| PMD | Increase of ≥25% in tumor ¹⁸F-FDG SUVₘₐₓ from baseline; visible increase (>20% |
| | of the maximum long axis) in extent of 18F-FDG tumor uptake or new ¹⁸F-FDG avid |
| | lesion; |
| SMD | Increase of <25% or decrease of <15% in tumor ¹⁸F-FDG SUVₘₐₓ uptake from |
| | baseline, or no increase in extent of ¹⁸F-FDG tumor uptake (>20% of the maximum |
| | long axis); |
| UE | FDG-PET examination is not available, or the status of target lesion cannot be |
| | determined; If one of the target lesions is considered non-evaluable, the rules for |
| | PMD are not applicable, and it cannot be identified as CMR, PMR or SMD, then the |
| | evaluation result will be UE unless unequivocal progression is established based on |
| | the evaluable target lesion. |

### Appendix VIII: PERCIST (PET Response Criteria in Solid Tumors) Evaluation Criteria

The PET Response Criteria in Solid Tumors evaluation criteria and overall response by PERCIST criteria are shown in Tables 21 and 22 below.

TABLE 21
PET Response PERCIST 1.0 Criteria.

| Criteria | PERCIST 1.0 |
|---|---|
| CMR | Disappearance of all lesions on PET images, lesions are indistinguishable from |
| | surrounding background blood pool levels and less than the mean liver SUL. |
| PMR | Reduction of ≥30% in measurable lesion SUL and absolute drop in SUL of at least 0.8 |
| | SUL units. |
| PMD | Increase of SUL ≥30% and absolute increase in SUL of 0.8 SUL unites from baseline, |
| | or development of novel lesion, or unequivocal progression (e.g., ≥30% increase) in |
| | non-target lesions, or unequivocal progression by RECIST. |
| SMD | Not conforming to other criteria |

TABLE 22
Overall Response by PERCIST 1.0 Criteria.

| Target Lesions | Non-target Lesions | New Lesions | Overall Response |
|---|---|---|---|
| CMR | CMR | No | CMR |
| CMR | Non-PMD | No | PMR |
| PMR | CMR/Non-PMD | No | PMR |
| SMD | CMR/Non-PMD | No | SMD |
| PMD | Any | Yes or No | PMD |
| Any | PMD | Yes or No | PMD |
| Any | Any | Yes | PMD |

TABLE 23
List of Abbreviations.

| Abbreviations | Description |
|---|---|
| ADA | Anti-Drug Antibodies |
| AE | Adverse Event |
| ALB | Albumin |
| ALT | Alanine Aminotransferase |
| ALP | Alkaline Phosphatase |
| ANC | Absolute Neutrophil Count |
| APTT | Activated Partial Thromboplastin Time |
| AST | Aspartate Aminotransferase |
| AUC₀₋ₜ | Area under the Curve from Zero to the Time Point |
| AUC_{0-inf} | Area under the Curve from Zero to Infinite |
| BMD | Bone Mineral Density |
| BUN | Blood Urea Nitrogen |
| Ca | Calcium |
| Cl | Chlorine |
| CL | Clearance of Drug |
| Cr | Creatinine |
| CRF | Case Report Form |
| Cₘₐₓ | Maximum Concentration |
| Cx | Connexin |
| CxDy | Cycle X Day Y |
| DBil | Direct Bilirubin |
| DCR | Disease Control Rate |
| DLT | Dose Limited Toxicity |
| DNA | Deoxyribonucleic Acid |
| DXA | Dual Energy X-ray Absorptiometry |
| DF | Degree of Fluctuation |
| FAS | Full Analysis Set |
| GCP | Good Clinical Practice |
| GGT | Glutamyltransferase |
| h | Hour |
| Hb | Hemoglobin |
| HIV | Human Immunodeficiency Virus |
| INR | International Normalized Ratio |
| IHC | Immunohistochemistry |
| ITT | Intend to Treat |
| K | Potassium |
| LVEF | Left Ventricular Ejection Fraction |
| Mg | Magnesium |
| Min | Minute |
| MTD | Maximum Tolerated Dose |
| Na | Sodium |
| NAbs | Neutralizing Antibodies |
| NOAEL | No Observable Adverse Effect Level |
| NYHA | New York Heart Association |
| ORR | Objective Response Rate |
| P | Phosphorus |
| PFS | Progression-Free-Survival |
| PFSR | Progression-Free-Survival Rate |
| PLT | Platelet |
| PK | Pharmacokinetics |
| PT | Prothrombin Time |
| IHC | Immunohistochemistry |
| qPCR | Quantitative Polymerase Chain Reaction |
| RBC | Red Blood Cell |
| RNA | Ribonucleic Acid |
| RP2D | Recommended Phase 2 Dose |
| SAE | Serious Adverse Event |
| SOP | Standard Operation Procedure |
| SRE | Skeletal Related Events |
| SS | Safety Analysis Set |
| TBil | Total Bilirubin |
| TTR | Time to Response |
| t_{1/2} | Half-life |
| Tₘₐₓ | Time to Maximum Plasma Concentration |
| Vd | Volume of Distribution |
| ULN | Upper Limit of Normal |
| UREA | Urea |
| WBC | White Blood Cell |

TABLE 24
Sequence Table.

| SEQ | | |
|---|---|---|
| ID | | |
| NO | Description | Amino Acid Sequence |
| 1 | ALMB-0168 | GYTFTSYY |
| | HCDR1 | |
| 2 | ALMB-0168 | INPSNAGT |
| | HCDR2 | |
| 3 | ALMB-0168 | TREGNPYYTMNY |
| | HCDR3 | |
| 4 | ALMB-0168 | QSLLESDGKTY |
| | LCDR1 | |
| 5 | ALMB-0168 | LVS |
| | HCDR2 | |
| 6 | ALMB-0168 | WQGTHFPWT |
| | HCDR3 | |
| 7 | ALMB-0168 VH | EVQLVQSGAEVKKPGASVKVSCKASGYTFTSYYMYWVRQAPGQGLEWIGGINPSNAGTNFNEKFKNRATLT |
| | | VDKSTSTAYMELSSLRSEDTAVYYCTREGNPYYTMNYWGQGTLVTVSS |
| 8 | ALMB-0168 VL | DVVMTQSPLSLPVTIGQPASISCKSSQSLLESDGKTYLNWLQQRPGQSPRRLIYLVSKLDSGVPDRFSGSG |
| | | SGTDFTLKISRVEAEDVGVYYCWQGTHFPWTFGGGTKVEIK |
| 9 | Heavy chain of | EVQLVQSGAEVKKPGASVKVSCKASGYTFTSYYMYWVRQAPGQGLEWIGGINPSNAGTNFNEKFKNRATLT |
| | ALMB-0168 | VDKSTSTAYMELSSLRSEDTAVYYCTREGNPYYTMNYWGQGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCVVVDVSQEDPEVQFNWYVDGVEVH |
| | | NAKTKPREEQFNSTYRVVSVLTVLHQDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDGSFFLYSRLTVDKSRWQEGNVFSCSV |
| | | MHEALHNHYTQKSLSLSLGK |
| 10 | Light chain of | DVVMTQSPLSLPVTIGQPASISCKSSQSLLESDGKTYLNWLQQRPGQSPRRLIYLVSKLDSGVPDRFSGSG |
| | ALMB-0168 | SGTDFTLKISRVEAEDVGVYYCWQGTHFPWTFGGGTKVEIKRTVAAPSVFIFPPSDEQLKSGTASVVCLLN |
| | | NFYPREAKVQWKVDNALQSGNSQESVTEQDSKDSTYSLSSTLTLSKADYEKHKVYACEVTHQGLSSPVTKS |
| | | FNRGEC |
| 11 | Heavy chain of | EVQLVQSGAEVKKPGASVKVSCKASGYTFTSYYMYWVRQAPGQGLEWIGGINPSNAGTNFNEKFKNRATLT |
| | Ab#L | VDKSTSTAYMELSSLRSEDTAVYYCTREGNPYYTMNYWGQGTLVTVSSASTKGPSVFPLAPSSKSTSGGTA |
| | | ALGCLVKDYFPEPVTVSWNSGALTSGVHTFPAVLQSSGLYSLSSVVTVPSSSLGTQTYICNVNHKPSNTKV |
| | | DKKVEPKSCDKTHTCPPCPAPEAAGGPSVFLFPPKPKDTLMISRTPEVTCVVVDVSHEDPEVKFNWYVDGV |
| | | EVHNAKTKPREEQYNSTYRVVSVLTVLHQDWLNGKEYKCKVSNKALPAPIEKTISKAKGQPREPQVYTLPP |
| | | SRDELTKNQVSLTCLVKGFYPSDIAVEWESNGQPENNYKTTPPVLDSDGSFFLYSKLTVDKSRWQQGNVFS |
| | | CSVMHEALHNHYTQKSLSLSPGK |
| 12 | Heavy chain of | EVQLVQSGAEVKKPGASVKVSCKASGYTFTSYYMYWVRQAPGQGLEWIGGINPSNAGTNFNEKFKNRATLT |
| | Ab#P | VDKSTSTAYMELSSLRSEDTAVYYCTREGNPYYTMNYWGQGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNTKV |
| | | DKRVESKYGPPCPPCPAPEFEGGPSVFLFPPKPKDTLMISRTPEVTCVVVDVSQEDPEVQFNWYVDGVEVH |
| | | NAKTKPREEQFNSTYRVVSVLTVLHQDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDGSFFLYSRLTVDKSRWQEGNVFSCSV |
| | | MHEALHNHYTQKSLSLSLGK |
| 13 | Heavy chain of | EVQLVQSGAEVKKPGASVKVSCKASGYTFTSYYMYWVRQAPGQGLEWIGGINPSNAGTNFNEKFKNRATLT |
| | Ab#T | VDKSTSTAYMELSSLRSEDTAVYYCTREGNPYYTMNYWGQGTLVTVSSASTKGPSVFPLAPCSRSTSESTA |
| | | ALGCLVKDYFPEPVTVSWNSGALTSGVHTFPAVLQSSGLYSLSSVVTVPSSSLGTKTYTCNVDHKPSNTKV |
| | | DKRVESKYGPPCPPCPAPEAAGGPSVFLFPPKPKDTLMISRTPEVTCVVVDVSQEDPEVQFNWYVDGVEVH |
| | | NAKTKPREEQFNSTYRVVSVLTVLHQDWLNGKEYKCKVSNKGLPSSIEKTISKAKGQPREPQVYTLPPSQE |
| | | EMTKNQVSLTCLVKGFYPSDIAVEWESNGQPENNYKTTPPVLDSDGSFFLYSRLTVDKSRWQEGNVFSCSV |
| | | MHEALHNHYTQKSLSLSLGK |
| 14 | Epitope sequence | FLSRPTEKTI |
| | of Ab#H | |

1. A method of treating a bone cancer in a subject in need thereof, comprising administering to the subject a therapeutically effective amount of an anti-connexin 43 (Cx43) antibody, or antigen binding fragment thereof, wherein the anti-Cx43 antibody comprises
a heavy chain variable region comprising:
an HCDR1 having the amino acid sequence of SEQ ID NO 1;
an HCDR2 having the amino acid sequence of SEQ ID NO 2; and
an HCDR3 having the amino acid sequence of SEQ ID NO 3; and
a light chain variable region comprising:
an LCDR1 having the amino acid sequence of SEQ ID NO 4;
an LCDR2 having the amino acid sequence of SEQ ID NO 5; and
an LCDR3 having the amino acid sequence of SEQ ID NO 6;
wherein the subject is at least 2 years old and less than 18 years old.

2. The method of claim 1, wherein the subject is
(i) at least 3 years old, at least 4 years old, at least 5 years old, at least 6 years old, at least 7 years old, at least 8 years old, at least 9 years old, at least 10 years old, at least 11 years old, at least 12 years old, at least 13 years old, at least 14 years old, at least 15 years old, at least 16 years old, or at least 17 years old; or
(ii) 12 years old, or more than 12 years old.

3. (canceled)

4. The method of claim 1, wherein the therapeutically effective amount of the anti-Cx43 antibody, or antigen binding fragment thereof, is
(i) about 0.001 mg/kg to about 300 mg/kg, or
(ii) about 1 mg/kg, about 3 mg/kg, about 6 mg/kg, about 12 mg/kg, about 18 mg/kg, about 24 mg/kg, about 30 mg/kg, or about 36 mg/kg.

5. (canceled)

6. (canceled)

7. The method of claim 1, wherein the anti-Cx43 antibody, or antigen binding fragment thereof, is administered
(i) about once every 2 weeks, about once every 3 weeks, or about once every 4 weeks, or
(ii) about once every 3 weeks.

8. (canceled)

9. The method of claim 1, wherein
(j) the anti-Cx43 antibody, or antigen binding fragment thereof, is administered intravenously, or subcutaneously;
(ii) the administration of the anti-Cx43 antibody, or antigen binding fragment thereof, is completed within about 5 minutes, about 10 minutes, about 15 minutes, about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more;
(iii) the anti-Cx43 antibody, or antigen binding fragment thereof, is administered in a pharmaceutically acceptable composition, wherein the pharmaceutically acceptable composition comprises a pharmaceutically acceptable carrier;
(iv) the anti-Cx43 antibody is a humanized antibody; or
(v) the anti-Cx43 antibody, or antigen binding fragment thereof, enhances Cx43 hemichannel opening in the subject.

10.-12. (canceled)

13. The method of claim 1, wherein the anti-Cx43 antibody, or antigen binding fragment thereof, comprises a heavy chain variable region comprising the amino acid sequence of SEQ ID NO 7, and/or a light chain variable region comprising the amino acid sequence of SEQ ID NO 8.

14. The method of claim 1, wherein the anti-Cx43 antibody comprises a heavy chain comprising the amino acid sequence of any one of SEQ ID NOS 9 and 11-13, and/or a light chain comprising the amino acid sequence of SEQ ID NO 10.

15. (canceled)

16. (canceled)

17. The method of claim 1, wherein the bone cancer is osteosarcoma.

18. The method of claim 17, wherein the osteosarcoma is
(i) resectable, or unresectable;
(ii) stage I-A, stage I-B, stage II-A, stage II-B, stage III, stage IV-A, or stage IV-B;
(iii) low grade, or high grade; and/or
(iv) primary osteosarcoma, osteoblastic osteosarcoma, chondroblastic osteosarcoma, fibroblastic osteosarcoma, small cell osteosarcoma, telangiectatic osteosarcoma, parosteal osteosarcoma, periosteal osteosarcoma, classic osteosarcoma, osteoblastic osteosarcoma-sclerosing type osteosarcoma, chondroblastoma-like osteosarcoma, chondromyxoid fibroma-like osteosarcoma, clear-cell osteosarcoma, malignant fibrous histiocytoma-like osteosarcoma, giant cell rich osteosarcoma, and/or epithelioid osteosarcoma.

19. The method of claim 1, wherein the subject had failed treatment with
(i) at least one standard of care for osteosarcoma comprising chemotherapy, surgery, immune therapy, or radiotherapy, or a combination thereof; or
(ii) methotrexate, doxorubicin, cisplatin, and ifosfamide, apatinib, anlotinib, vindesine, vincristine, docetaxel, paclitaxel, irinotecan, bortezomib, albumin-bound paclitaxel, nedaplatin (Aqupla), pemetrexed, etoposide, gemcitabine, lobaplatin, recombinant human endostatin, eribulin, dacarbazine, pazopanib, immune checkpoint inhibitors, surgery, or radiotherapy, or the combination thereof.

20. (canceled)

21. The method of claim 1, wherein after treatment with the anti-Cx43 antibody, or antigen binding fragment thereof, the subject achieves:
(i) progression-free survival for at least 1 month, 2 months, 3 months, at least 4 months, at least 5 months, or at least 6 months;
(ii) improved overall survival by at least 1 month, at least 2 months, at least 3 months, at least 4 months, at least 5 months, at least 6 months;
(iii) improved EQ-5D score;
(iv) improved disease progression as assessed by PERCIST criteria, RECIST criteria, and/or ICDS criteria;
(v) improved pain score as assessed by NRS;
(vi) reduced tumor size by at least 1%, at least 2%, at least 3%, at least 5%, at least 8%, at least 10%, at least 15%, at least 20%, or at least 30%; and/or
(vii) complete or partial response.

22. (canceled)

23. A method of treating osteosarcoma in a subject in need thereof, comprising administering to the subject a therapeutically effective amount of an anti-connexin 43 (Cx43) antibody, or antigen binding fragment thereof, wherein the anti-Cx43 antibody comprises
a heavy chain variable region comprising:
an HCDR1 having the amino acid sequence of SEQ ID NO 1;
an HCDR2 having the amino acid sequence of SEQ ID NO 2; and
an HCDR3 having the amino acid sequence of SEQ ID NO 3; and
a light chain variable region comprising:
an LCDR1 having the amino acid sequence of SEQ ID NO 4;
an LCDR2 having the amino acid sequence of SEQ ID NO 5; and
an LCDR3 having the amino acid sequence of SEQ ID NO 6;
wherein the subject is at least 12 years old and less than 18 years old.

24. (canceled)

25. The method of claim 23, wherein the therapeutically effective amount of the anti-Cx43 antibody, or antigen binding fragment thereof, is about 1 mg/kg, about 3 mg/kg, about 6 mg/kg, about 12 mg/kg, about 18 mg/kg, about 24 mg/kg, about 30 mg/kg, or about 36 mg/kg.

26. (canceled)

27. The method of claim 23, wherein the anti-Cx43 antibody, or antigen binding fragment thereof, is administered
(i) about once every 2 weeks, about once every 3 weeks, or about once every 4 weeks; or
(ii) about once every 3 weeks.

28. (canceled)

29. The method of claim 23, wherein
(i) the anti-Cx43 antibody, or antigen binding fragment thereof, is administered intravenously;
(ii) the administration of the anti-Cx43 antibody, or antigen binding fragment thereof, is completed within about 5 minutes, about 10 minutes, about 15 minutes about 20 minutes, about 25 minutes, about 30 minutes, about 40 minutes, about 50 minutes, about 60 minutes, about 70 minutes about 80 minutes, about 90 minutes, about 100 minutes, about 110 minutes, about 120 minutes, about 150 minutes, about 180 minutes, or more;
(iii) the anti-Cx43 antibody, or antigen binding fragment thereof, is administered in a pharmaceutically acceptable composition, wherein the pharmaceutically acceptable composition comprises a pharmaceutically acceptable carrier;
(iv) the anti-Cx43 antibody is a humanized antibody; or
(v) the anti-Cx43 antibody, or antigen binding fragment thereof, enhances Cx43 hemichannel opening in the subject.

30.-32. (canceled)

33. The method of claim 23, wherein the anti-Cx43 antibody, or antigen binding fragment thereof, comprises a heavy chain variable region comprising the amino acid sequence of SEQ ID NO 7, and/or a light chain variable region comprising the amino acid sequence of SEQ ID NO 8.

34. The method of claim 23, wherein the anti-Cx43 antibody comprises a heavy chain comprising the amino acid sequence of any one of SEQ ID NOS 9 and 11-13, and/or a light chain comprising the amino acid sequence of SEQ ID NO 10.

35.-37. (canceled)

38. The method of claim 23, wherein the osteosarcoma is
(i) resectable, or unresectable;
(ii) stage I-A, stage I-B, stage II-A, stage II-B, stage III, stage IV-A, or stage IV-B;
(iii) low grade, or high grade; and/or
(iv) primary osteosarcoma, osteoblastic osteosarcoma, chondroblastic osteosarcoma, fibroblastic osteosarcoma, small cell osteosarcoma, telangiectatic osteosarcoma, parosteal osteosarcoma, periosteal osteosarcoma, classic osteosarcoma, osteoblastic osteosarcoma-sclerosing type osteosarcoma, chondroblastoma-like osteosarcoma, chondromyxoid fibroma-like osteosarcoma, clear-cell osteosarcoma, malignant fibrous histiocytoma-like osteosarcoma, giant cell rich osteosarcoma, and/or epithelioid osteosarcoma.

39. The method of claim 23, wherein the subject had failed treatment with
(i) at least one standard care of osteosarcoma comprising chemotherapy, surgery, immune therapy, or radiotherapy, or a combination thereof; or
(ii) methotrexate, doxorubicin, cisplatin, and ifosfamide, apatinib, anlotinib, vindesine, vincristine, docetaxel, paclitaxel, irinotecan, bortezomib, albumin-bound paclitaxel, nedaplatin (Aqupla), pemetrexed, etoposide, gemcitabine, lobaplatin, recombinant human endostatin, eribulin, dacarbazine, pazopanib, immune checkpoint inhibitors, surgery, or radiotherapy, or the combination thereof.

40. (canceled)

41. The method of any one of claims 1-40, wherein after treatment with the anti-Cx43 antibody, or antigen binding fragment thereof, the subject achieves:
(i) progression-free survival for at least 1 month, 2 months, 3 months, at least 4 months, at least 5 months, or at least 6 months;
(ii) improved overall survival by at least 1 month, at least 2 months, at least 3 months, at least 4 months, at least 5 months, at least 6 months;
(iii) improved EQ-5D score;
(iv) improved disease progression as assessed by PERCIST criteria, RECIST criteria, and/or ICDS criteria;
(v) improved pain score as assessed by NRS;
(vi) reduced tumor size by at least 1%, at least 2%, at least 3%, at least 5%, at least 8%, at least 10%, at least 15%, at least 20%, or at least 30%; and/or
(vii) complete or partial response.

42. (canceled)


SEQUENCE LISTING (14 sequences)

SEQ ID NO: 1 - protein, 8 aa, unidentified
GYTFTSYY

SEQ ID NO: 2 - protein, 8 aa, unidentified
INPSNAGT

SEQ ID NO: 3 - protein, 12 aa, unidentified
TREGNPYYTMNY

SEQ ID NO: 4 - protein, 11 aa, unidentified
QSLLESDGKTY

SEQ ID NO: 5 - skipped

SEQ ID NO: 6 - protein, 9 aa, unidentified
WQGTHFPWT

SEQ ID NO: 7 - protein, 119 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSS

SEQ ID NO: 8 - protein, 112 aa, unidentified
DVVMTQSPLSLPVTIGQPASISCKSSQSLLESDGKTYLNWLQQRPGQSPRRLIYLVSKLDSGVPDRFSGSGSGTDFTLKISRVEAEDVGVYYCWQGTHFPWTFGGGTKVEIK

SEQ ID NO: 9 - protein, 446 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, unidentified
DVVMTQSPLSLPVTIGQPASISCKSSQSLLESDGKTYLNWLQQRPGQSPRRLIYLVSKLDSGVPDRFSGSGSGTDFTLKISRVEAEDVGVYYCWQGTHFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 449 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 446 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 446 aa, unidentified
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 10 aa, unidentified
FLSRPTEKTI